(19) United States
(12) Patent Application Publication
(10) Pub. No.: US 2024/0327826 A1
(43) Pub. Date: Oct. 3, 2024

(54) COMPOSITIONS AND METHODS FOR IMPROVING T CELL PERSISTENCE AND FUNCTION

(71) Applicant: The Board of Trustees of the Leland Stanford Junior University, Stanford, CA (US)

(72) Inventors: Ansuman Satpathy, Stanford, CA (US); Julia BELK, Stanford, CA (US)

(21) Appl. No.: 18/580,472

(22) PCT Filed: Jul. 28, 2022
(86) PCT No.: PCT/US22/74251

§ 371 Date: Jan. 18, 2024

Related U.S. Application Data
(60) Provisional application No. 63/226,559, filed on Jul. 28, 2021.

(51) Int. Cl. C12N 15/10; C12N 5/0783; C12N 9/22; C12N 15/11; C12N 15/90

(52) U.S. Cl. CPC C12N 15/1082; C12N 5/0636; C12N 9/22; C12N 15/11; C12N 15/907; C12N 2310/20; C12N 2503/00

(57) ABSTRACT

Provided herein are compositions comprising T cells modified to remove genes which play a role in T cell exhaustion, and compositions and methods thereof. Engineered T cells lacking at least one gene selected from the group consisting of: INO80C, GATA3, ARID1A, WDR82, TRP53, GPR137C, ZFP219, HDAC1, ELMSAN1, and ACTR8 are provided. Engineered T cells lacking at least one gene encoding a chromatin remodeling protein, including INO80 nucleosome positioning complex proteins and SWI/SNF family members (e.g., BAF complex proteins) are also provided. Methods are provided to treat a disease or disorder in a subject comprising administration of the engineered T cells. Also provided are methods for preventing exhaustion of engineered T cells, methods of making a therapeutic T cell and methods of identifying genes which play a role in T cell exhaustion.

### CROSS-REFERENCE TO RELATED APPLICATIONS

This application claims the benefit of U.S. Provisional Application No. 63/226,559, filed Jul. 28, 2021, the content of which is herein incorporated by reference in its entirety.

### STATEMENT REGARDING FEDERALLY SPONSORED RESEARCH OR DEVELOPMENT

This invention was made with government support under grant number CA230188 awarded by the National Institutes of Health. The government has certain rights in the invention.

### FIELD

The present disclosure relates to modified T cells and composition and methods of use thereof.

### SEQUENCE LISTING STATEMENT

The contents of the electronic sequence listing titled (STDU2-39684-601.xml; Size: 7,214 bytes; and Date of Creation: Jul. 28, 2022) is herein incorporated by reference in its entirety.

### BACKGROUND

The development of T cell exhaustion is a major barrier to durable and effective CAR-T cell therapies, particularly for solid tumors. Upon cancer recognition, chronic T cell activation by tumor cells leads to T cell exhaustion, which results in impaired proliferation, cytotoxicity, and effector functions, thereby limiting T cell killing of cancer cells. Exhaustion is often targeted with checkpoint inhibitors. However, a majority of patients fail to respond to these agents and no efficacy has been shown in combination with CAR T cells in clinical trials.

### SUMMARY

Provided herein are engineered T cells lacking at least one gene which facilitates or supports T cell persistence and functionality. In some embodiments, the engineered T cell maintains functionality under conditions in which non-engineered T cells display exhaustion.

In some embodiments, the engineered T cells lack at least one gene selected from the group consisting of: INO80C, GATA3, ARID1A, WDR82, TRP53, GPR137C, ZFP219, HDAC1, ELMSAN1, and ACTR8. In some embodiments, the engineered T cells lack two or more genes selected from the group consisting of: INO80C, GATA3, ARID1A, WDR82, TRP53, GPR137C, ZFP219, HDAC1, ELMSAN1, and ACTR8.

In some embodiments, the engineered T cell lacks at least one chromatin remodeling protein or a gene encoding thereof. In some embodiments, the engineered T cell lacks two or more chromatin remodeling proteins or a genes encoding thereof. In some embodiments, the at least one chromatin remodeling protein is a INO80 nucleosome positioning complex protein or SWI/SNF family member, or a combination thereof. In some embodiments, the INO80 nucleosome positioning complex protein is Actr5, Ino80, Ino80c, Ino80b, Actr8, or a combination thereof. In some embodiments, the SWI/SNF family member is a member of cBAF (canonical BRG1/BRM-associated factor) complex. In some embodiments, the SWI/SNF family member is Arid1a, Arid2, Arid1b, Smarcb1, Smarcd2, Smarca4, Smarcc1, or a combination thereof. In some embodiments, the engineered T cell further lacks at least one gene selected from the group consisting of: GATA3, WDR82, TRP53, GPR137C, ZFP219, HDAC1, and ELMSAN1.

The engineered T cells may further comprise an exogenous receptor or a nucleic acid encoding thereof. In some embodiments, the exogenous receptor is a T cell receptor (TCR) or chimeric antigen receptor (CAR). In some embodiments, the exogenous receptor is specific for a tumor antigen.

In some embodiments, the T cells are derived from a biological sample from a subject. In some embodiments, the T cells are isolated from a tumor sample. In some embodiments, the T cells are expanded ex vivo.

Also provided herein are compositions comprising a population of the engineered T cells described herein. The compositions may further comprise at least one therapeutic agent. In some embodiments, the at least one therapeutic agent is selected from the group consisting of: an agent for treating T cell exhaustion; an antiviral agent; an antibiotic agent; an antimicrobial agent; a chemotherapeutic agent; or a combination thereof.

Further provided herein are methods of making a therapeutic T cell. In some embodiments, the methods comprise comprising obtaining a sample comprising T cells; altering the DNA of the T cells to knockout or disrupt at least one gene selected from the group consisting of: INO80C, GATA3, ARID1A, WDR82, TRP53, GPR137C, ZFP219, HDAC1, ELMSAN1, and ACTR8; and engineering the T cells to express an exogenous receptor.

In some embodiments, the methods comprise obtaining a sample comprising T cells; altering the DNA of the T cells to knockout or disrupt at least one gene encoding a chromatin remodeling protein; and engineering the T cells to express an exogenous receptor. In some embodiments, the chromatin remodeling protein is a INO80 nucleosome positioning complex protein or SWI/SNF family member, or a combination thereof. In some embodiments, the INO80 nucleosome positioning complex protein is Actr5, Ino80, Ino80c, Ino80b, Actr8, or a combination thereof. In some embodiments, the SWI/SNF family member is a member of cBAF complex. In some embodiments, the SWI/SNF family member is Arid1a, Arid2, Arid1b, Smarcb1, Smarcd2, Smarca4, Smarcc1, or a combination thereof. In some embodiments, the method further comprises altering the DNA of the T cells to knockout or disrupt at least one gene selected from the group consisting of: GATA3, WDR82, TRP53, GPR137C, ZFP219, HDAC1, and ELMSAN1.

In some embodiments, altering the DNA prevents or reduces exhaustion of the T cells.

In some embodiments, the T cells are derived from a biological sample from a subject. In some embodiments, the T cells are isolated from a tumor sample. In some embodiments, the T cells are expanded ex vivo.

In some embodiments, the exogenous receptor is a T cell receptor (TCR) or chimeric antigen receptor (CAR). In some embodiments, the exogenous receptor is specific for a tumor antigen.

Additionally provided are methods for treating a disease or disorder in a subject comprising administering to the subject an effective amount of the engineered T cells or compositions thereof described herein. In some embodiments, the disease or disorder comprises an infection or cancer. In some embodiments, the cancer comprises a tumor.

In some embodiments, the administering reduces the number of cancerous cells in the subject, reduces and/or eliminates the tumor burden in the subject, and/or shows enhanced cancer treatment compared to administration of unmodified T cells.

In some embodiments, the method further comprises administering at least one additional therapeutic agent. The at least one therapeutic agent may be selected from the group consisting of: an agent for treating T cell exhaustion; an antiviral agent; an antibiotic agent; an antimicrobial agent; a chemotherapeutic agent; or a combination thereof.

In some embodiments, the T cells are autologous to the subject.

In some embodiments, the T cells maintain functionality under conditions in which non-engineered T cells display exhaustion and/or have improved persistence and function compared to non-engineered T cells.

Provided herein are methods of preventing T cell exhaustion. In some embodiments, the methods comprise genetically modifying the T cell to lack at least one gene selected from the group consisting of: INO80C, GATA3, ARID1A, WDR82, TRP53, GPR137C, ZFP219, HDAC1, ELMSAN1, and ACTR8.

In some embodiments, the methods comprise genetically modifying the T cell to lack at least one gene encoding a chromatin remodeling protein. In some embodiments, the chromatin remodeling protein is a INO80 nucleosome positioning complex protein or SWI/SNF family member, or a combination thereof. In some embodiments, the INO80 nucleosome positioning complex protein is Actr5, Ino80, Ino80c, Ino80b, Actr8, or a combination thereof. In some embodiments, the SWI/SNF family member is a member of cBAF complex. In some embodiments, the SWI/SNF family member is Arid1a, Arid2, Arid1b, Smarcb1, Smarcd2, Smarca4, Smarcc1, or a combination thereof. In some embodiments, the method further comprises genetically modifying the T cell to lack at least one gene selected from the group consisting of: GATA3, WDR82, TRP53, GPR137C, ZFP219, HDAC1, and ELMSAN1.

In some embodiments, the T cells have increased survival in the presence of a chronic antigen.

The engineered T cells may further comprise an exogenous receptor or a nucleic acid encoding thereof. In some embodiments, the exogenous receptor is a T cell receptor (TCR) or chimeric antigen receptor (CAR). In some embodiments, the exogenous receptor is specific for a tumor antigen.

The methods may further comprise administering the T cells to a subject in need thereof. In some embodiments, the subject has cancer or an infectious disease.

Provided herein are methods for screening for genes which facilitate T cell exhaustion comprising: culturing T cells under conditions of chronic or acute stimulation for at least six days, wherein each of T cells comprises at least one gene knockout or knockdown; isolating T cells not showing an exhausted T cell surface phenotype; and identifying the at least one gene knockout or knockdown.

In some embodiments, the T cells are a T cell library, wherein the T cell library comprises at least one T cell with a knockout or knockdown for each gene in the genome of the T cell. In some embodiments, the T cells are CD8+ T cells. In some embodiments, the T cells are isolated from a subject.

In some embodiments, the T cells are generated using a CRISPR-Cas system wherein each cell comprises at least one guide RNA directed to a gene of interest.

In some embodiments, conditions of chronic stimulation comprise culturing the T cells using anti-CD3 coated plates. In some embodiments, the conditions of chronic stimulation further comprise culturing the T cells with IL-2. In some embodiments, conditions of acute stimulation comprise culturing the T cells with IL-2. In some embodiments, the culturing lasts six to ten days.

Also provided herein are systems or kits comprising engineered T cells as described herein or a system for genetic engineering T cells. The system for genetic engineering T cells may comprise a clustered interspersed short palindromic repeat (CRISPR)/CRISPR-associated protein (Cas) system, or a nucleic acid(s) encoding thereof, as described herein. In certain embodiments, the system for genetic engineering T cells comprises Cas9 (e.g., dCas9), or a nucleic acid encoding Cas9, and a gRNA directed to at least one gene which facilitates T cell exhaustion, or a nucleic acid encoding the gRNA.

In some embodiments, the at least one gene which facilitates T cell exhaustion may be selected from the group consisting of: INO80C, GATA3, ARID1A, WDR82, TRP53, GPR137C, ZFP219, HDAC1, ELMSAN1, and ACTR8.

In some embodiments, the at least one gene which facilitates T cell exhaustion is a gene encoding a chromatin remodeling protein. In some embodiments, the chromatin remodeling protein is a INO80 nucleosome positioning complex protein or SWI/SNF family member, or a combination thereof. In some embodiments, the INO80 nucleosome positioning complex protein is Actr5, Ino80, Ino80c, Ino80b, Actr8, or a combination thereof. In some embodiments, the SWI/SNF family member is a member of cBAF complex. In some embodiments, the SWI/SNF family member is Arid1a, Arid2, Arid1b, Smarcb1, Smarcd2, Smarca4, Smarcc1, or a combination thereof.

In some embodiments, the systems or kits further comprise an exogenous receptor or a nucleic acid encoding thereof.

In some embodiments, the systems or kits further comprise at least one additional therapeutic agent. The at least one therapeutic agent may be selected from the group consisting of: an agent for treating T cell exhaustion; an antiviral agent; an antibiotic agent; an antimicrobial agent; a chemotherapeutic agent; or a combination thereof.

Other aspects and embodiments of the disclosure will be apparent in light of the following detailed description.

### BRIEF DESCRIPTION OF THE DRAWINGS

FIGS. 1A-1F show an exemplary in vitro assay recapitulating epigenetic hallmarks of T cell exhaustion. FIG. 1A is a diagram of in vitro exhaustion assay. FIG. 1B shows the surface phenotype of CD8+ T cells at day 0 and day 10 of the T cell exhaustion assay, gated on live cells. FIG. 1C is principal component analysis of ATAC-seq profiles of CD8+ T cells throughout the course of chronic stimulation. FIG. 1D is Pdcd1 and Entpd1 gene loci for a representative replicate of each time point in the in vitro exhaustion assay, as well as previously published reference ATAC-seq profiles from T cells in tumors or LCMV (Miller et al., (2019) Nat. Immunol. 20, 326-336). FIG. 1E is a heatmap showing ATAC-seq coverage of each peak in the “Terminal Exhaustion peak set” for each time point in the in vitro exhaustion assay. Reference data from TILs is also included. Selected nearest genes are indicated on the right. FIG. 1F is the chromVAR motif accessibility heatmap for each ATAC-seq sample. Selected motifs are indicated on the right. Top 100 most variable motifs are shown.

FIGS. 2A-2F show genome-wide functional interrogation of T cell exhaustion. FIG. 2A is a diagram of genome-wide T cell exhaustion screen. FIG. 2B shows the correlation of replicate screens with selected functional categories of genes colored as indicated. Gene sets were based on GO Terms and were supplemented with manual annotations. FIG. 2C is a volcano plot with top hits labeled. FIG. 2D is individual sgRNA residuals for top hits in different functional categories: integrin or TCR signaling. FIG. 2E is a GO Term analysis of the top 100 hits. FIG. 2F is individual sgRNA residuals for top hits in different functional categories: chromatin (left), selected receptors and transcription factors (center), or other (right). Histogram for all guide residuals is shown above, and 1000 randomly selected guides are shown in the background of each row in gray, for visual reference.

FIG. 3 is cytoscape network representation of top hits. Top positive and negative hits from the genome-wide screen are shown. Each protein is represented by a node in the cytoscape network, colored by its z-score in the genome-wide screen. Nodes are connected if there is a high confidence protein-protein interaction.

FIGS. 4A-4G show the targeted follow up of top hits in vivo. FIG. 4A is a diagram of exemplary in vivo pooled screening. FIG. 4B is a volcano plot of genes enriched or depleted in MC-38 tumors. FIG. 4C shows the correlation of tumor LFC z-scores to spleen LFC z-scores colored by functional category. FIG. 4D shows the correlation of in vivo z-score and in vitro (genome-wide) z-scores for genes in the minipool. FIG. 4E is a cytoscape protein-protein interaction network colored by z-score in MC-38 tumors. FIG. 4F is a boxplot of tumor vs input log fold change for each sgRNA targeting the indicated gene, with the mean control log fold change subtracted. FIG. 4G is z-scores for every tumor for the top 15 in vivo hits as well as control guides and Cd3d.

FIGS. 5A-5G show in vivo Perturb-seq of tumor infiltrating lymphocytes. FIG. 5A is a diagram of direct capture Perturb-seq of sorted TILs. FIG. 5B is scRNA-seq profiles of TILs colored by cluster assignment. FIG. 5C, left is scRNA-seq profiles of cells colored by the perturbation detected in each cell. Cells where no guide, or multiple guides, were detected per cell are shown in grey. 5C, right is z-score of each gene knockout in vitro and in vivo (MC-38 tumor z-score). FIG. 5D is the correlation of genome wide gene expression differences of cells with each perturbation compared to control cells. Gene expression profiles were averaged across cells and then subtracted. FIG. 5E is an upset plot of induced genes for different perturbations. FIG. 5F is an upset plot of repressed genes for different perturbations. FIG. 5G shows selected genes, grouped by category, and the change in expression relative to control in different perturbations.

FIGS. 6A-6F show additional characterization of in vitro assay. FIG. 6A shows the surface phenotype of chronically stimulated T cells throughout the in vitro exhaustion assay. FIG. 6B is a graph of the proliferation of chronically stimulated and acutely stimulated T cells in vitro. FIG. 6C shows effector cytokine production of acutely (left) and chronically (right) stimulated T cells. Cells were restimulated with PMA and ionomycin 8 days after initial stimulation. FIG. 6D is a graph of the survival of B16 cells after co-culture with acutely or chronically stimulated OT-1 T cells. Tumor cells were pulsed with cognate peptide (SIINFEKL-SEQ ID NO: 1). FIG. 6E is a graph of B16-ovalbumin tumor growth in vivo after adoptive transplant of acutely or chronically stimulated T cells. FIG. 6F is a heatmap showing ATAC-seq coverage of each peak in the “Terminal Exhaustion peak set” for each time point in the in vitro exhaustion assay. Reference data from T cells in LCMV is also included.

FIGS. 7A-7D show quality control data for in vitro genome wide screen. FIG. 7A shows expression of BFP on day 2 of screen. FIG. 7B shows the surface phenotype of cells before gDNA extraction. FIG. 7C shows sgRNA representation of each sample. FIG. 7D are graphs of the guide count correlations (Acute vs Chronic) for each replicate. CD3 subunits are shown in red, all other guides in black.

FIGS. 8A-8D show LCMV Clone 13 expression analysis of top hits. FIG. 8A is a graph of cell types identified in scRNA-seq data. FIG. 8B shows expression of Pdcd1, Havcr2, Tcf7, and Cx3cr1 in single cells. FIG. 8C is violin plots of the expression of the gene module containing the top 100 in vitro hits across clusters. FIG. 8D is a cytoscape network of top hits colored by average expression across all single cells.

FIGS. 9A-9D show additional data for targeted in vivo screening. FIG. 9A are graphs of tumor sizes and T cell injection timeline for each group in the minipool screen. FIG. 9B, top is a boxplot of spleen vs input log fold change for each sgRNA targeting the indicated gene, with the mean control log fold change subtracted. FIG. 9B, bottom is a heatmap of z-scores for every spleen for the top 15 in vivo hits as well as control guides and Cd3d. FIG. 9C is GO Term analysis of the top 20 positive hits in tumors. FIG. 9D is LCMV expression analysis for 9 top epigenetics hits.

FIGS. 10A-10E show additional data for in vivo direct capture Perturb-seq. FIG. 10A is violin plots showing expression of Pdcd1, Havcr2, and Mki67 by cluster. FIG. 10B is a volcano plot comparing Cluster 1 (C1) to Cluster 2 (C2). Selected differential genes are indicated. FIG. 10C shows expression of selected transcription factors Tbx21, Tox, Eomes, and Tcf7. FIG. 10D is visualization of cells containing each gene knockout. FIG. 10E, top is a graph of counts of repressed or induced genes for each perturbation. FIG. 10E, bottom is a graph of counts of repressed or induced genes that are shared with Nr4a3-KO for each perturbation.

FIG. 11A is a casTLE volcano plot of the Chronic vs Acute stimulation screen comparison, with top hits labeled. FIG. 11 B shows the correlation of Acute vs Chronic z-scores in the mini-pool versus the genome-wide screen. FIG. 11C shows the correlation of the mini-pool Chronic vs Acute z-scores against Acute vs Input (left) or Chronic vs Input (right). Genes in (G) and (H) are colored by functional category: TCR signaling, integrin signaling (orange), chromatin (blue), or other (grey). Colored boxes in (H, left) denote enhanced, similar, or reduced expansion (top to bottom) after acute stimulation.

FIG. 12A shows the correlation of tumor LFC z-scores to spleen LFC z-scores, colored by functional category. FIG. 12B shows the correlation of in vivo z-score and in vitro z-scores for genes in the CRISPR mini-pool. FIG. 12C shows the correlation of in vivo MC-38 and B16 tumor z-scores for genes in the CRISPR mini-pool. FIG. 12D is a cytoscape protein-protein interaction network colored by z-score in MC-38 tumors. FIG. 12E, top is a boxplot of tumor versus input log fold change for each sgRNA targeting the indicated gene, with the mean control log fold change subtracted. FIG. 12E, bottom is heatmaps showing the sgRNA average of the indicated in vivo or in vitro screen for the same hits. FIG. 12 F is individual sgRNA residuals for six top hits showing the Tumor vs Input comparison (left), Spleen vs Input (center), and in vitro Chronic vs Acute (right).

FIGS. 13A-13F show SWI/SNF mini-pool CRISPR screens and functional studies demonstrating that tuning cBAF activity can enhance anti-tumor immunity. FIG. 13A shows an in vitro competition assay of Arid1a-sgRNA versus CTRL1 T cells. Left: cells were mixed on Day 4 at the indicated ratios and passaged in the chronic stimulation assay for 6 days. On Day 10, proliferation relative to CTRL1 T cells and surface phenotype were assessed by flow cytometry. FIG. 13B shows an in vivo competition assay of Arid1a-sgRNA versus CTRL1 T cells. Cells were mixed on Day 6 (input) and then transplanted into tumor bearing mice. On Day 15, relative proliferation in the tumor was assessed by flow cytometry. FIG. 13C are graphs of tumor sizes for each cohort. Statistical significance was assessed at Day 15. FIG. 13D are survival graphs showing that Arid1a-sgRNA T cells significantly improve survival of tumor-bearing mice. FIG. 13E shows the correlation of SWI/SNF CRISPR mini-pool tumor enrichments in MC-38 versus B16 tumor models. FIG. 13F are cartoons of the three BAF complexes colored by z-score from SWI/SNF CRISPR mini-pool experiments in MC-38 tumors. BAF complex cartoons adapted from (Mashtalir et al., 2018). * p<0.05, *** p<0.001.

FIGS. 14A-14D show conserved function of ARID1A in human T cells in vitro and in vivo. FIG. 14A are graphs of the proliferation and viability of primary human T cells after electroporation of the indicated RNP. Left: Acutely stimulated T cells. Right: Chronically stimulated T cells using anti-CD3-coated plates. Data shown is representative of 3 independent experiments and 3 donors. FIG. 14B is a schematic of CRISPR mini-pool screen in primary human CD8⁺ T cells transduced with the NY-ESO-1-specific TCR, 1G4. FIG. 14C shows the results of the human CRISPR mini-pool screen aggregated by gene. FIG. 14D shows the results of the human CRISPR mini-pool screen with individual sgRNA replicates shown as dots. Genes are ordered from highest to lowest average LFC. Results shown in (FIGS. 14C and 14D) are combined from 2 independent donors, 2 mice per donor, and 2 sgRNAs per target gene, for a total of n=8 sgRNA replicates per target gene. In (FIGS. 14C and 14D), orange indicates inhibitory receptors, red indicates TCR signaling pathway genes, blue indicates chromatin remodelers and grey indicates negative controls.

FIGS. 15A-15G show in vivo Perturb-seq revealing distinct transcriptional roles of the cBAF and INO80 complexes in TILs. FIG. 15A is a schematic of direct-capture Perturb-seq of sorted TILs. FIG. 15B is scRNA-seq profiles of TILs colored by cluster assignment. FIG. 15C is scRNA-seq profiles of cells colored by the perturbation detected in each cell. Cells where no guide, or multiple guides, were detected are shown in grey. FIG. 15D shows the expression of selected marker genes in each single cell. FIG. 15E is the analysis of LCMV signature gene sets for each cluster. Gene set enrichment scores were calculated for each single cell, cell values were averaged by cluster and z-scored. FIG. 15F is histograms of Pearson correlation of gene expression differences of pairs of sgRNAs. Top: Pairs targeting the same gene are shown in blue, other pairs are shown in gray. Bottom: Pairs targeting the same protein complex are shown in red, other pairs are shown in gray. Complexes considered in the analysis are cBAF (Arid1a, Arid/b, Smarcd2, and Smarcc1) and INO80 (Ino80c and Actr5). Pairs of sgRNAs that target the same gene are excluded. FIG. 15G, Left: Heatmap of the correlation of gene expression differences of each pair of sgRNAs. FIG. 15G, Center (from left to right): Representation of each sgRNA in the pre-transplant sample, cell count of each sgRNA in the Perturb-seq dataset, and estimated fold change of each sgRNA relative to controls. FIG. 15G, Right: Proportion of cells in each cluster for each sgRNA.

FIGS. 16A-16H show cBAF-depleted T cells exhibit enhanced effector gene signatures and reduced terminal exhaustion. FIG. 16A is volcano plots comparing aggregated cells with the indicated perturbation versus CTRL1 cells. FIG. 16B is pairwise correlations of gene expression differences induced by each perturbation. FIG. 16C is heatmaps of all upregulated (up) or downregulated (down) genes in at least one perturbation, grouped by which perturbation has the strongest effect on expression. Selected genes in each block are labeled. FIG. 16D shows a comparison of upregulated or downregulated gene sets by perturbation of cBAF subunits, Arid1a, Smarcd2, or Smarcc1. FIG. 16E shows a comparison of gene sets up- or downregulated by perturbation of INO80 subunits Actr5, or Ino80c. FIG. 16F shows a comparison of gene sets upregulated by perturbation of cBAF subunits, INO80 subunits, or Pdcd1, Gata3, or Arid2. FIG. 16G is enrichments of upregulated and downregulated gene sets in LCMV expression data (Daniel et al., 2021). Module scores of each gene set were computed for each single cell in the LCMV dataset, averaged by cluster, and then z-scored to obtain the indicated enrichment z-scores. FIG. 16H shows selected GO Terms of indicated gene sets.

FIGS. 17A-17F show Arid1a facilitates the acquisition of the exhausted T cell chromatin state. FIG. 17A shows principal component analysis of ATAC-seq profiles of Arid1a-sgRNA and CTRL1 cells in the in vitro exhaustion competition assay. Unperturbed naïve and activated samples (Day 0 and 2) are included for reference. FIG. 17B shows a comparison of ‘opened’ and ‘closed’ ATAC-seq peak sets from Day 6 to Day 10 for each genotype. FIG. 16C is visualization of ‘opened’ and ‘closed’ ATAC-seq peak sets, with selected nearest genes labeled. FIG. 16D shows ATAC-seq signal tracks of selected gene loci. Representative replicates are shown for each condition. FIG. 16E is heatmaps showing ATAC-seq coverage of each peak in the “Terminal Exhaustion peak set” for Arid1a-sgRNA and CTRL1 cells at Day 6 and Day 10 in the in vitro exhaustion assay. Reference data from TILs is also included, as well as reference naïve and activated cell profiles. FIG. 16F is chromVAR motif accessibility heatmap for Arid1a-sgRNA and CTRL1 ATAC-seq samples. Selected motifs are indicated on the right. Top 100 most variable motifs are shown.

FIG. 18A shows effector cytokine production of acutely (left) and chronically (right) stimulated T cells after 6 days of chronic stimulation (day 8 after isolation). Cells were restimulated with PMA and ionomycin 8 days after initial stimulation. FIG. 18B are graphs of empirical cumulative distribution of peak accessibility for peaks in the Term. T_{EX} peak set (top) and Prog. T_{EX} peak set (bottom) for the indicated time points in vitro. Reference profiles from TILs are included as indicated. FIG. 18C is box plots for the indicated peak sets in the in vitro exhaustion assay and reference TIL samples. Each dot represents one peak.

FIGS. 19A and 19B are comparisons of cytokine production after acute stimulation, chronic stimulation (6 days of anti-CD3 stimulation) (FIG. 19A), or the modified chronic stimulation protocol (6 days of anti-CD3 stimulation after a 48-hour rest) (FIG. 19B). FIG. 19C are Gini index and empirical cumulative distribution function shown for each sample in the genome-wide screen.

FIGS. 20A-20E show the comparison of CRISPR analysis strategies. FIG. 20A is volcano plots of genome wide CRISPR screen results using casTLE (top left), MAGeCK (top right), and the disclosed pipeline (bottom). FIG. 20B shows comparison of hit lists for each of the three pipelines. FIG. 20C shows comparison of LFC difference computed by the disclosed pipeline to the casTLE Effect (left) and MAGeCK LFC (right). FIG. 20D is a counts table for Rpl13a. FIG. 20E shows genome wide screen results when z-scores are computed relative to all sgRNAs or a set of olfactory receptors (Vmnr* genes).

FIGS. 21A-21E show data for targeted in vitro screening. FIG. 21A is sgRNA representation of each sample in the in vitro mini-pool screen. FIG. 21B shows the correlation of the sgRNA counts of each sample in the mini-pool screen. FIG. 21C shows the correlation of the Chronic vs Acute replicate z-scores. FIG. 21D is a cytoscape interaction network with genes colored by their z-score in the Chronic vs Acute mini-pool screen. FIG. 21E is a cytoscape interaction network with genes colored by their fitness categorization in acute stimulation.

FIGS. 22A-22F show data for targeted in vivo screening and validation of Arid1a-targeting sgRNAs. FIG. 22A shows sgRNA pool coverage for each sample in the in vivo mini-pool screen. FIG. 22B is sgRNA residuals in tumors, spleens, and in vitro mini-pool Chronic vs Acute for selected genes in the “TCR signaling” and “Integrin signaling” categories. FIG. 22C is boxplot of spleen vs input and acute vs chronic log fold change for each sgRNA targeting the indicated gene, with the mean control log fold change subtracted. FIG. 22D is Sanger sequencing (TIDE) analysis of editing efficiency of Arid1a sgRNAs. FIG. 22E is Western blot analysis of protein knockdown for Arid1a sgRNAs, as well as Arid1b and Smarca4 expression. FIG. 22F shows quantification of protein knockdown for each identified isoform of Arid1a (panel C three bands). * p<0.05, ** p<0.01, *** p<0.001, **** p<0.0001.

FIGS. 23A-23G show data on the in vivo Perturb-seq. FIG. 23A is scRNA-seq profiles of TILs colored by each independent experiment. FIG. 23B is scRNA-seq profiles of TILs colored by each sample. FIG. 23C is scRNA-seq profiles of TILs colored by predicted phase of the cell cycle. FIG. 23D shows additional marker genes shown for each cluster. FIG. 23E shows expanded reference LCMV dataset with single cell profiles colored by LCMV cluster. FIG. 23F shows expanded LCMV dataset with single cell profiles colored by LCMV infection (Acute corresponds to Armstrong infection while Chronic corresponds to Clone 13) and time point (Day 8 or Day 21 post infection). FIG. 23G is a heatmap of the correlation of gene expression differences subsetted on each cluster. The indicated gene knockdown was compared to CTRL1 cells within each cluster. Comparisons with <150 cells in the comparison groups are excluded due to lack of statistical power.

FIGS. 24A-24G show data on up- and down-regulated gene sets and ATAC-seq data. FIG. 24A shows a comparison of gene sets downregulated by perturbation of cBAF subunits, INO80 subunits, or Pdcd1-sgRNA, Gata3-sgRNA, or Arid2-sgRNA. FIG. 24B is module scores of the indicated gene sets computed for each cell in the expanded LCMV reference dataset. FIG. 24C is box plots for the indicated peak sets in the in vitro exhaustion assay and reference TIL samples. Each dot represents one peak. FIG. 24D are graphs of empirical cumulative distribution of peak accessibility for peaks in the Term. T_{EX} peak set (top) and Prog. T_{EX} peak set (bottom) for the indicated samples in vitro. Reference profiles from TILs are included as indicated. FIG. 24E is principal component analysis of ATAC-seq data of primary human T cells chronically stimulated for six days. FIG. 24F shows differential peaks between ARID1A-sgRNA and AAVS primary human T cells. FIG. 24G is HOMER analysis of TF motifs enriched in AAVS ‘up’ peaks. Selected highly ranked motifs are shown. Results in (FIGS. 24E-24G) are merged from three different human donors in two independent experiments with two different ARID1A targeting sgRNAs per donor.

### DETAILED DESCRIPTION

Herein, an in vitro T cell exhaustion model enabled genome-wide screening for genes that influence T cell function. Using this model and genome-wide CRISPR screens, several gene targets were identified, deletion of which: prevented CAR-T cell exhaustion, improved T cell survival in the presence of chronic antigen in vitro, and improved T cell persistence and function in tumor models in vivo. These included several genes involved in gene regulation and epigenetic modifications, including ARID1A, WDR82, INO80, HDAC1, and ZFP219. Cell therapies with deletions of each of these genes find use for improved CAR-T or other adoptive T cell based therapies.

Section headings as used in this section and the entire disclosure herein are merely for organizational purposes and are not intended to be limiting.

### 1. Definitions

The terms “comprise(s),” “include(s),” “having,” “has,” “can.” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The terms “engineered,” “non-naturally occurring,” “modified,” and “synthetic” are used interchangeably and indicate the involvement of the hand of man. The terms, when referring to cells or nucleic acids mean that the nucleic acid or the cell is at least substantially free from at least one other component with which they are naturally associated in nature and as found in nature.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. The term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, the terms “providing,” “administering,” and “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

A “subject” or “patient” may be human or non-human and may include, for example, animal strains or species used as “model systems” for research purposes, such a mouse model as described herein. Likewise, subject may include either adults or juveniles (e.g., children). Moreover, subject may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, “treat,” “treating,” and the like means a slowing, stopping, or reversing of progression of a disease or disorder when provided an engineered T cell or composition described herein to an appropriate control subject. The term also means a reversing of the progression of such a disease or disorder to a point of eliminating or greatly reducing the symptoms. As such, “treating” means an application or administration of the engineered T cells or compositions described herein to a subject, where the subject has a disease or a symptom of a disease, where the purpose is to cure, heal, alleviate, relieve, alter, remedy, ameliorate, improve, or affect the disease or symptoms of the disease.

### 2. Engineered T Cells

Provided herein are engineered T cells lacking at least one gene which facilitates or supports T cell persistence and functionality. The genes may play a role in chromatin organization, chromatin remodeling (e.g., ATP-dependent chromatic remodeling), T cell receptor signaling pathways, immune response-activating signal transduction, immune response-activating cell surface receptor signaling pathways, nucleosome disassembly, and/or Fc receptor signaling pathways. In some embodiments, the genes comprise chromatin remodeling and transcription factors.

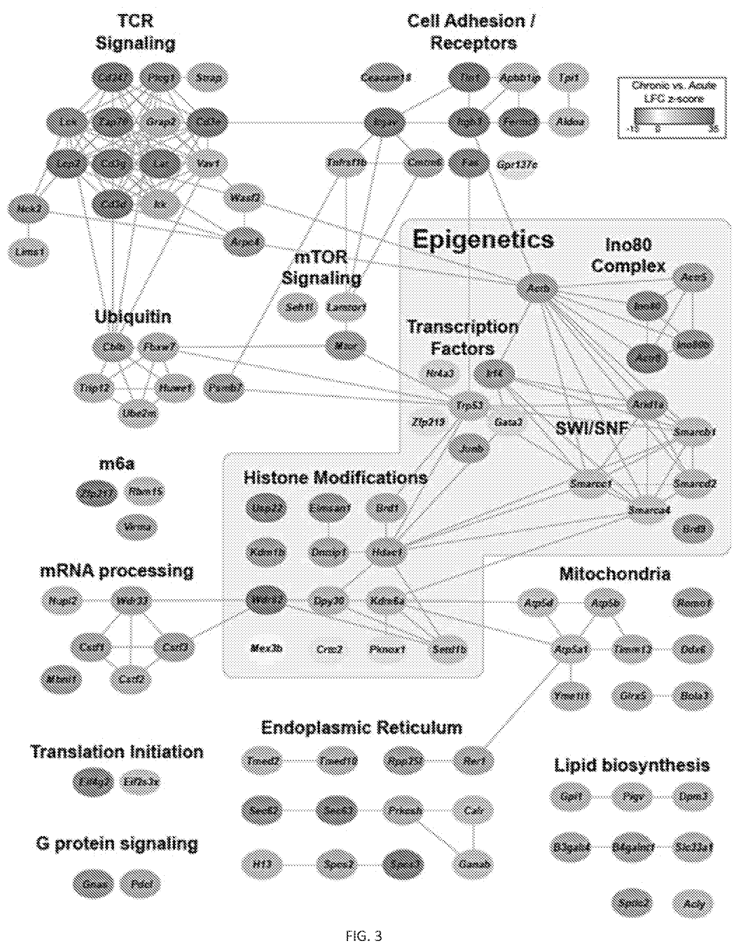

In some embodiments, the at least one gene is selected from those included in FIGS. 3, 5C, and 9B. In certain embodiments, the at least one gene is: INO80C (INO80 Complex Subunit C), GATA3 (GATA Binding Protein 3), ARID1A (AT-Rich Interaction Domain 1A), WDR82 (WD Repeat Domain 82), TRP53 (Tumor Protein P53), GPR137C (G Protein-Coupled Receptor 137C), ZFP219 (Zinc Finger Protein 219), HDAC1 (Histone Deacetylase 1), ELMSAN1 (ELM2 and Myb/SANT-like domain containing 1), or ACTR8 (Actin Related Protein 8). In some embodiments, the engineered T cell lacks two or more of: INO80C, GATA3, ARID1A, WDR82, TRP53, GPR137C, ZFP219, HDAC1, ELMSAN1, and ACTR8. In some embodiments, the engineered T cell further lacks NR4A3 (Nuclear Receptor Subfamily 4 Group A Member 3).

In some embodiments, the engineered T cell lacks at least one chromatin remodeling protein or a gene encoding thereof. In some embodiments, the engineered T cell lacks two or more chromatin remodeling proteins or a genes encoding thereof. In some embodiments, the at least one chromatin remodeling protein is a INO80 nucleosome positioning complex protein or SWI/SNF (SWitch/Sucrose Non-Fermentable) family member, or a combination thereof. In some embodiments, the INO80 nucleosome positioning complex protein is Actr5 (Actin Related Protein 5), Ino80 (INO80 Complex ATPase Subunit), Ino80c (INO80 Complex Subunit C), Ino80b (INO80 Complex Subunit B), Actr8 (Actin Related Protein 8), or a combination thereof. In some embodiments, the SWI/SNF family member is a member of cBAF complex. In some embodiments, the SWI/SNF family member is Arid1a (AT-Rich Interaction Domain 1A). Arid2 (AT-Rich Interaction Domain 2), Arid1b (AT-Rich Interaction Domain 1B), Smarcb1 (SWI/SNF related, matrix associated, actin dependent regulator of chromatin, subfamily b, member 1), Smarcd2 (SWI/SNF related, matrix associated, actin dependent regulator of chromatin, subfamily d, member 2), Smarca4 (SWI/SNF related, matrix associated, actin dependent regulator of chromatin, subfamily a, member 4), Smarcc1 (SWI/SNF related, matrix associated, actin dependent regulator of chromatin, subfamily c, member 1), or a combination thereof. In some embodiments, the engineered T cell further lacks at least one gene selected from the group consisting of: GATA3, WDR82, TRP53, GPR137C, ZFP219, HDAC1, and ELMSAN1.

“Lacking a gene” can refer to either a full or partial deletion, mutation, or other disruption that results in no functional gene product being expressed or being targeted for degradation immediately upon expression. Thus, lacking a gene may result from any disruption to the genetic code such that a portion of the gene is altered, thereby affecting transcription and/or translation, e.g., rendering the gene unreadable through knockout techniques or by insertion of an additional gene for a desired protein or insertion of a regulatory sequence that modulates transcription of an existing sequence. In certain embodiments, the gene or a portion thereof is deleted, commonly referred to as gene knockout.

Any method known in the art for genetic engineering may be used to generate the engineered T cells described herein, including, but not limited to, use of a clustered interspersed short palindromic repeat (CRISPR)/CRISPR-associated protein (Cas) system, a meganuclease, transcription activator-like effector nuclease (TALEN) or a Zinc-finger nuclease (ZFN).

In some embodiments, the T cells maintain functionality under conditions in which unmodified T cells, T cells not lacking at least one gene which facilitates or supports T cell persistence and functionality, display exhaustion (e.g., maintaining functionality of T cells exposed to excessive antigen). “T cell exhaustion” refers to loss of T cell function, which may occur as a result of an infection (e.g., a chronic infection) or a disease. T cell exhaustion is associated with increased expression of exhaustion markers and inhibitory receptors (e.g., PD-1, TIM-3, and LAG-3), apoptosis, and reduced cytokine secretion.

The invention is not limited by the type of T cell which is engineered to lack at least one gene which facilitates or supports T cell persistence and functionality. The T cells may be selected from CD3+ T cells (e.g., a combination of CD4+ and CD8+ T cells), CD8+ T cells, CD4+ T cells, natural killer (NK) T cells, alpha beta T cells, gamma delta T cells, or any combination thereof. In some embodiments, the T cells are memory T cells (e.g., central memory T cells or effector memory T cells). In some embodiments, the T cells are tumor infiltrating lymphocytes. In some embodiments, the T cells are cytokine-induced killer cells. In select embodiments, the T cells are CD8+ T cells.

In some embodiments, the T cells are naturally occurring T cells. For example, the T cells may be isolated from a subject sample. In some embodiments, the T cell is an anti-tumor T cell (e.g., a T cell with activity against a tumor (e.g., an autologous tumor) that becomes activated and expands in response to antigen). Anti-tumor T cells include, but are not limited to, T cells obtained from resected tumors or tumor biopsies (e.g., tumor infiltrating lymphocytes (TILs)) and a polyclonal or monoclonal tumor-reactive T cell (e.g., obtained by apheresis, expanded ex vivo against tumor antigens presented by autologous or artificial antigen-presenting cells). In some embodiments, the T cells are expanded ex vivo.

In some embodiments, the T cells further comprise an exogenous receptor or a nucleic acid encoding an exogenous receptor. In some embodiments, the exogenous receptor is a T cell receptor (TCR) or a chimeric antigen receptor (CAR).

The exogenous receptor is not limited by its specificity to recognize and respond to any specific antigen or protein. Such receptors are generally composed of extracellular domains comprising a specific antigen binding motif (e.g., single-chain antibody (scFv)) linked to intracellular T cell signaling motifs.

In certain embodiments, the T cells are genetically modified with exogenous receptors that recognize and respond to antigens for infectious disease and/or autoimmunity (e.g., Aspergillus carbohydrate β-glucan, Hepatitis C virus E2 glycoprotein, HIV envelope glycoprotein gp120).

In certain embodiments, the T cells are genetically modified with exogenous receptors that recognize and respond to tumor antigens. The invention is not limited by the type of tumor antigen so recognized. The term “tumor antigen” as used herein refers to any molecule (e.g., protein, peptide, lipid, carbohydrate, etc.) solely or predominantly expressed or over-expressed by a tumor cell or cancer cell, such that the antigen is associated with the tumor or cancer. The tumor antigen can additionally be expressed by normal, non-tumor, or non-cancerous cells. However, in such cases, the expression of the tumor antigen by normal, non-tumor, or noncancerous cells is not as robust as the expression by tumor or cancer cells. In this regard, the tumor or cancer cells can over-express the antigen or express the antigen at a significantly higher level, as compared to the expression of the antigen by normal, non-tumor, or noncancerous cells. Also, the cancer antigen can additionally be expressed by cells of a different state of development or maturation. For instance, the tumor antigen can be additionally expressed by cells of the embryonic or fetal stage, which cells are not normally found in an adult. Alternatively, the tumor antigen can be additionally expressed by stem cells or precursor cells, which cells are not normally found in an adult.

The tumor antigen can be an antigen expressed by any cell of any cancer or tumor. The tumor antigen may be a tumor antigen of only one type of cancer or tumor, such that the tumor antigen is associated with or characteristic of only one type of cancer or tumor. Alternatively, the tumor antigen may be a tumor antigen (e.g., may be characteristic) of more than one type of cancer or tumor. For example, the tumor antigen may be expressed by both breast and prostate cancer cells and not expressed at all by normal, non-tumor, or non-cancer cells.

Exemplary tumor antigens include, but are not limited to, glycoprotein 100 (gp100), melanoma antigen recognized by T cells 1 (MART-1), melanoma antigen gene (MAGE) Family Members (e.g., MAGE-A 1, MAGE-A2, MAGE-A3, MAGE-A4, MAGE-A5, MAGE-A6, MAGE-A7, MAGE-A8, MAGE-A9, MAGE-A10, MAGE-A11, MAGE-A12), New York esophageal squamous cell carcinoma 1 (NY-ESO-1), vascular endothelial growth factor receptor-2 (VEGFR-2), glioma-associated antigen, carcinoembryonic antigen (CEA), beta-human chorionic gonadotropin, alphafetoprotein (AFP), lectin-reactive AFP, thyroglobulin, human telomerase reverse transcriptase, prostate-specific antigen (PSA), prostate-carcinoma tumor antigen-1 (PCTA-1), insulin growth factor (IGF)-I, IGF-II, IGF-I receptor, intestinal carboxyl esterase, human epidermal growth factor receptor 2 (HER-2), mesothelin, and epidermal growth factor receptor variant III (EGFR III).

Any T cell containing a receptor that recognizes a tumor antigen finds use in the T cells, compositions, and methods of the invention. Examples include, but are not limited to, T cells expressing a receptor (e.g., a native or naturally occurring receptor, or a receptor engineered to express a synthetic receptor such as an engineered TCR or a CAR) that recognize an antigen selected from CD19, CD20, CD22, receptor tyrosine kinase-like orphan receptor 1 (ROR1), disialoganglioside 2 (GD2), Epstein-Barr Virus (EBV) protein or antigen, folate receptor, mesothelin, human carcinoembryonic antigen (CEA), prostatic acid phosphatase (PAP), CD33/IL3R, tyrosine protein kinase Met (c-Met) or hepatocyte growth factor receptor (HGFR), prostate-specific membrane antigen (PSMA), Glycolipid F77, epidermal growth factor receptor variant III (EGFRvIII), NY-ESO-1, melanoma antigen gene (MAGE) Family Member A3 (MAGE-A3), melanoma antigen recognized by T cells 1 (MART-1), GP1000, p53, or other tumor antigen described herein.

In some embodiments, the T cell is engineered to express a chimeric antigen receptor (CAR). Any CAR that binds with specificity to a desired antigen (e.g., tumor antigen) may be utilized with the present invention. In certain embodiments, the CAR comprises an antigen-binding domain. In certain embodiments, the antigen-binding domain is a single-chain variable fragment (scFv) containing heavy and light chain variable regions that bind with specificity to the desired antigen. In some embodiments, the CAR further comprises a transmembrane domain (e.g., a T cell transmembrane domain (e.g., a CD28 transmembrane domain)) and a signaling domain comprising one or more immunoreceptor tyrosine-based activation motifs (ITAMs) (e.g., a T cell receptor signaling domain (e.g., TCR zeta chain)). In some embodiments, the CAR comprises one or more co-stimulatory domains (e.g., domains that provide a second signal to stimulate T cell activation). The invention is not limited by the type of co-stimulatory domain. Indeed, any co-stimulatory domain known in the art may be used including, but not limited to, CD28, OX40/CD134, 4-1BB/CD137/TNFRSF9, the high affinity immunoglobulin E receptor-gamma subunit, FcERIγ, ICOS/CD278, interleukin 2 subunit beta (ILRβ) or CD122, cytokine receptor common subunit gamma (IL-2Rγ) or CD132, and CD40. In some embodiments, the co-stimulatory domain is 4-1BB. In some embodiments, the co-stimulatory domain is CD28.

The CAR may comprise a target-specific binding element otherwise referred to as an antigen binding moiety. The choice of moiety depends upon the type and number of ligands that define the surface of a target cell. For example, the antigen binding domain may be chosen to recognize a ligand that acts as a cell surface marker on target cells associated with a particular disease state. Examples of cell surface markers that may act as ligands for the antigen moiety domain in the CAR of the invention include those associated with viral, bacterial, and parasitic infections, autoimmune diseases and, as described above, cancer cells.

Depending on the desired antigen to be targeted, a CAR can be engineered to include the appropriate antigen binding moiety specific to the desired antigen target. For example, if CD19 is the desired antigen that is to be targeted, an antibody for CD19 can be used as the antigen binding moiety for incorporation into the CAR of the invention.

The nucleic acid encoding the exogenous receptor may comprise DNA or RNA (e.g., mRNA). In some embodiments, the nucleic acid comprises vectors.

The nucleic acid may comprise a promoter that is constitutive, regulatable or inducible, cell type specific, tissue-specific, or species specific. In addition to the sequence sufficient to direct transcription, the promoter may also include sequences of other regulatory elements that are involved in modulating transcription (e.g., enhancers, Kozak sequences and introns). Many promoter/regulatory sequences useful for driving constitutive expression are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), HI (human polymerase III RNA promoter), U6 (human U6 small nuclear promoter), and the like. Additional promoters that can be used for expression, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The present disclosure also provides for vectors containing the nucleic acid and cells containing the nucleic acid or vectors thereof.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Additionally, the vector may contain, for example, some or all of the following: a selectable marker gene for selection of stable or transient transfectants in host cells; transcription termination and RNA processing signals; 5′- and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into a cell, the vectors may be maintained as an autonomously replicating sequence or extrachromosomal element or may be integrated into host DNA. The nucleic acids may be delivered to the cells by any suitable means.

Viral and non-viral based gene transfer methods can be used to introduce the nucleic acids into cells. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells. Viral vectors include, for example, retroviral, lentiviral, adenoviral, adeno-associated and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

Vectors according to the present disclosure can be transformed, transfected, or otherwise introduced into cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment).

Additionally, delivery vehicles such as nanoparticle- and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

Also provided are compositions comprising a population of engineered T cells as described herein.

The composition may optionally include at least one additional therapeutic agent, such as other drugs for treating T cell exhaustion (e.g., anti-PD-1 checkpoint inhibitor, such as nivolumab), or other medications used to treat a subject for an infection or disease associated with T cell exhaustion (e.g., antiviral, antibiotic, antimicrobial, or anti-cancer drugs).

In some embodiments, the at least one additional therapeutic agent comprises at least one chemotherapeutic agent. As used herein, the term “chemotherapeutic,” “chemotherapeutic agent,” or “anti-cancer drug” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor (Everolimus), alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib. In select embodiments, the chemotherapeutic agent comprises paclitaxel.

The compositions can include, for example, cytokines, chemokines and other biologic signaling molecules, tumor specific vaccines, cellular cancer vaccines (e.g., GM-CSF transduced cancer cells), tumor specific monoclonal antibodies, autologous and allogeneic stem cell rescue (e.g., to augment graft versus tumor effects), other therapeutic antibodies, molecular targeted therapies, anti-angiogenic therapy, infectious agents with therapeutic intent (such as tumor localizing bacteria) and gene therapy.

The compositions may include pharmaceutically acceptable carriers. The term “pharmaceutically acceptable carrier,” as used herein, means a non-toxic, inert solid, semi-solid or liquid filler, diluent, encapsulating material, surfactant, cyclodextrins or formulation auxiliary of any type. A carrier may include a single ingredient or a combination of two or more ingredients. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, preservatives and antioxidants can also be present in the composition, according to the judgment of the formulator.

The route of administration and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis).

### 3. Methods of Making a Therapeutic T Cell

The present disclosure provides methods for making a therapeutic T cell.

In some embodiments, the methods comprise obtaining a sample of T cells; altering the DNA of the T cells to knockout or disrupt at least one gene selected from the group consisting of: INO80C, GATA3, ARID1A, WDR82, TRP53, GPR137C, ZFP219, HDAC1, ELMSAN1, and ACTR8; and engineering the T cells to express an exogenous receptor.

In some embodiments, the methods comprise obtaining a sample comprising T cells; altering the DNA of the T cells to knockout or disrupt at least one gene encoding a chromatin remodeling protein; and engineering the T cells to express an exogenous receptor. In some embodiments, the chromatin remodeling protein is a INO80 nucleosome positioning complex protein or SW/SNF family member, or a combination thereof. In some embodiments, the INO80 nucleosome positioning complex protein is Actr5, Ino80, Ino80c, Ino80b, Actr8, or a combination thereof. In some embodiments, the SWI/SNF family member is a member of cBAF complex. In some embodiments, the SWI/SNF family member is Arid1a, Arid2, Arid1b, Smarcb1, Smarcd2, Smarca4, Smarcc1, or a combination thereof. In some embodiments, the method further comprises altering the DNA of the T cells to knockout or disrupt at least one gene selected from the group consisting of: GATA3, WDR82, TRP53, GPR137C, ZFP219, HDAC1, and ELMSAN1.

Altering the DNA prevents or reduces exhaustion of the T cells as compared with cells not including the modification. Thus, altering the DNA increases T cell persistence and function, thereby, improving the T cell for therapeutic uses.

The T cells may be selected from CD3+ T cells (e.g., a combination of CD4+ and CD8+ T cells), CD8+ T cells, CD4+ T cells, natural killer (NK) T cells, alpha beta T cells, gamma delta T cells, or any combination thereof. In some embodiments, the T cells are memory T cells (e.g., central memory T cells or effector memory T cells). In some embodiments, the T cells are tumor infiltrating lymphocytes. In some embodiments, the T cells are cytokine-induced killer cells. In select embodiments, the T cells are CD8+ T cells.

In some embodiments, the T cells are naturally occurring T cells. For example, the T cells may be isolated from a subject sample. In some embodiments, the T cell is an anti-tumor T cell (e.g., a T cell with activity against a tumor (e.g., an autologous tumor) that becomes activated and expands in response to antigen). Anti-tumor T cells include, but are not limited to, T cells obtained from resected tumors or tumor biopsies (e.g., tumor infiltrating lymphocytes (TILs)) and a polyclonal or monoclonal tumor-reactive T cell (e.g., obtained by apheresis, expanded ex vivo against tumor antigens presented by autologous or artificial antigen-presenting cells). In some embodiments, the T cells are expanded ex vivo.

Altering the DNA of the T cells to knockout or disrupt at least one gene may use methods known in the art and described elsewhere herein.

Engineering the T cells to express an exogenous receptor may comprise transfecting, transforming, or otherwise introducing a nucleic acid into the cell which expresses an exogenous receptor. Nucleic acids and methods for transfecting, transforming, or otherwise introducing such nucleic acids into a cell described elsewhere herein are suitable for the disclosed method.

In some embodiments, the exogenous receptor is a T cell receptor (TCR) or a chimeric antigen receptor (CAR). The exogenous receptor is not limited by its specificity to recognize and respond to any specific antigen or protein. In certain embodiments, the T cells are genetically modified with exogenous receptors that recognize and respond to antigens for infectious disease and/or autoimmunity. In certain embodiments, the T cells are genetically modified with exogenous receptors that recognize and respond to tumor antigens

### 4. Methods of Treatment

The present disclosure also provides methods for treating a disease or disorder.

In some embodiments, the methods comprise administering to the subject an effective amount of T cells modified to lack at least one gene which facilitates or supports T cell persistence and functionality.

In certain embodiments, the at least one gene is selected from the group consisting of: INO80C, GATA3, ARID1A, WDR82, TRP53, GPR137C, ZFP219, HDAC1, ELMSAN1, and ACTR8.

In certain embodiments, the at least one gene encodes a chromatin remodeling protein. In some embodiments, the chromatin remodeling protein is a INO80 nucleosome positioning complex protein or SWI/SNF family member, or a combination thereof. In some embodiments, the INO80 nucleosome positioning complex protein is Actr5, Ino80, Ino80c, Ino80b, Actr8, or a combination thereof. In some embodiments, the SWI/SNF family member is a member of cBAF complex. In some embodiments, the SWI/SNF family member is Arid1a, Arid2, Arid1b, Smarcb1, Smarcd2, Smarca4, Smarcc1, or a combination thereof.

The invention is not limited by the type of disease or condition treated. Any disease or condition that is treatable via administration of T cells can be treated in an improved and more effective manner using T cells and compositions thereof as described herein.

In some embodiments, the administration inhibits or reduces T cell exhaustion (e.g., compared to a subject receiving the same amount of T cells (e.g., CAR T cells or T cells comprising an exogenous TCR) not engineered to lack the at least one gene. In some embodiments, the at least one gene is selected from the group consisting of: INO80C, GATA3, ARID1A, WDR82, TRP53, GPR137C, ZFP219, HDAC1, ELMSAN1, and ACTR8. In some embodiments, the chromatin remodeling protein is a INO80 nucleosome positioning complex protein or SW/SNF family member, or a combination thereof. In some embodiments, the INO80 nucleosome positioning complex protein is Actr5, Ino80, Ino80c, Ino80b, Actr8, or a combination thereof. In some embodiments, the SWI/SNF family member is a member of cBAF complex. In some embodiments, the SWI/SNF family member is Arid1a, Arid2, Arid1b, Smarcb1, Smarcd2, Smarca4, Smarcc1, or a combination thereof.

In some embodiments, the administration results in improved T cell survival in the presence of chronic antigen and/or improved T cell persistence and function compared to non-engineered T cells.

The T cells may be isolated from a subject. In some embodiments, the T cells are allogeneic to the subject. In some embodiments, the T cells are autologous to the subject. Thus, the T cells may be isolated from a sample from the subject, modified and expanded ex vivo, and returned to the subject.

In some embodiments, the disease or condition is cancer. In some embodiments, the disease or condition is an infectious disease. The invention is not limited by the type of cancer or by the type of infectious disease. Indeed, any cancer known in the art for which T cell therapy is used for treatment may be treated with the compositions and methods of the invention. Similarly, any infectious disease known in the art for which T cell therapy is used for treatment may be treated with the compositions and methods of the invention.

In certain embodiments, the invention provides methods for treating or delaying the progression of cancer, or for treating or delaying the progress of infectious disease, in an individual comprising administering to the individual an effective amount of engineered T cells or compositions thereof, as described herein. In some embodiments, the treatment results in a sustained response in the individual after cessation of the treatment.

The methods can be used with any cancer cell or in a subject having any type of cancer, for example those described by the National Cancer Institute. In some embodiments, the cancer may be a carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, muscle tissue, ovary, pancreas, prostate, skin, spleen, stomach, testicle, thyroid, or uterus. In some embodiments, the cancer comprises a solid tumor. In some embodiments, the cancer is metastatic cancer.

The methods described herein may find use in treating conditions where enhanced immunogenicity is desired such as increasing tumor immunogenicity for the treatment of cancer. In some embodiments, the recombinant receptor (e.g., CAR and/or TCR) is specific for the cancer being treated. In some embodiments, the recombinant receptor (e.g., CAR and/or TCR) is generic for all cancers.

In certain embodiments, the present invention demonstrates that treatment of a subject having cancer with a therapeutically effective amount of the disclosed compositions is superior to treatment of a subject having cancer with unmodified T cells. In some embodiments, treatment with therapeutically effective amounts of the disclosed T cells or compositions thereof inhibits the development or growth of cancer cells or and/or renders the cancer cells as a population more susceptible to other treatments (e.g., the cell death-inducing activity of cancer therapeutic drugs or radiation therapies). Accordingly, T cells, compositions, and methods of the invention may be used as a monotherapy (e.g., to kill cancer cells, and/or reduce or inhibit cancer cell growth, induce apoptosis and/or cell cycle arrest in cancer cells), or when administered in combination with one or more additional agent(s), such as other anti-cancer agents (e.g., cell death-inducing or cell cycle-disrupting cancer therapeutic drugs or radiation therapies) to render a greater proportion of the cancer cells susceptible to killing, inhibited cancer cell growth, induced apoptosis and/or cell cycle arrest compared to the corresponding proportion of cells in an animal treated only with the cancer therapeutic drug or radiation therapy alone.

In some embodiments, the individual has cancer that is resistant (e.g., has been demonstrated to be resistant) to one or more other forms of anti-cancer treatment (e.g., chemotherapy, immunotherapy, etc.). In some embodiments, resistance includes recurrence of cancer or refractory cancer. Recurrence may refer to the reappearance of cancer, in the original site or a new site, after treatment. In some embodiments, resistance includes progression of the cancer during treatment with chemotherapy. In some embodiments, resistance includes cancer that does not respond to traditional or conventional treatment with a chemotherapeutic agent. The cancer may be resistant at the beginning of treatment or it may become resistant during treatment. In some embodiments, the cancer is at early stage or at late stage.

In some embodiments, the modified T cells and compositions thereof are used to treat, ameliorate, or prevent a cancer that is characterized by resistance to one or more conventional cancer therapies (e.g., those cancer cells which are chemoresistant, radiation resistant, hormone resistant, and the like). In some embodiments, the treatment may inhibit the growth of resistant cancer cells outright and/or render such cells as a population more susceptible to cancer therapeutic drugs or radiation therapies (e.g., to the cell death-inducing activity thereof).

In certain embodiments, the therapeutically effective amount of the modified T cell composition reduces the number of cancer cells in the subject following such treatment. In certain embodiments, the therapeutically effective amount of the modified T cell composition reduces and/or eliminates the tumor burden in the subject following such treatment.

A wide range of second therapies may be used in conjunction with the methods of the present disclosure. The second therapy may be administration of an additional therapeutic agent or may be a second therapy not connected to administration of another agent. Such second therapies include, but are not limited to, surgery, immunotherapy, radiotherapy, or an additional chemotherapeutic or anti-cancer agent.

The second therapy may be administered at the same time as the initial therapy, either as a single composition or in a separate composition administered at substantially the same time as the initial therapy. In some embodiments, the second therapy may precede or follow the treatment of the first therapy by time intervals ranging from hours to months.

In certain embodiments, the method further comprises administering radiation therapy to the subject. In certain embodiments, the radiation therapy is administered before, at the same time as, and/or after the subject receives the therapeutically effective amount of the modified T cell composition.

In certain embodiments, the method further comprises administering to the subject one or more anticancer agents and/or one or more chemotherapeutic agents. In certain embodiments, the one or more anticancer agents and/or one or more chemotherapeutic agents are administered before, at the same time as, and/or after the subject receives the therapeutically effective amount of the engineered T cells or a composition thereof. In certain embodiments, combination treatment of a subject with a therapeutically effective amount of engineered T cells and a course of an anticancer agent produces a greater tumor response and clinical benefit in such subject compared to those treated with the engineered T cells or anticancer drugs/radiation alone. Since the doses for all approved anticancer drugs and radiation treatments are known, the present invention contemplates the various combinations of them with the engineered T cells.

In some embodiments, the second therapy comprises administration of antibodies. The antibodies may target antigens either specifically expressed by tumor cells or antigens shared with normal cells. In some embodiments, the antibody may target, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L1. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab and the like). In some embodiments, the additional therapeutic agent may comprise anti-PD-1/PD-L1 antibodies, including, but not limited to, pembrolizumab, nivolumab, cemiplimab, atezolizumab, avelumab, durvalumab, and ipilimumab. The antibodies may also be linked to a chemotherapeutic agent. Thus, in some embodiments, the antibody is an antibody-drug conjugate.

The administration of second therapy may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof.

### 5. Methods of Preventing T Cell Exhaustion

The present disclosure also provides methods preventing exhaustion (e.g., maintaining functionality of T cells exposed to excessive antigen) of engineered T cells. In some embodiments, the methods comprise genetically modifying the T cell to lack at least one gene selected from the group consisting of: INO80C, GATA3, ARID1A, WDR82, TRP53, GPR137C, ZFP219, HDAC1, ELMSAN1, and ACTR8. In some embodiments, the methods comprise genetically modifying the T cell to lack at least one gene encoding a chromatin remodeling protein. In some embodiments, the chromatin remodeling protein is a INO80 nucleosome positioning complex protein or SWI/SNF family member, or a combination thereof. In some embodiments, the INO80 nucleosome positioning complex protein is Actr5, Ino80, Ino80c, Ino80b, Actr8, or a combination thereof. In some embodiments, the SWI/SNF family member is a member of cBAF complex. In some embodiments, the SWI/SNF family member is Arid1a, Arid2. Arid1b, Smarcb1, Smarcd2, Smarca4, Smarcc1, or a combination thereof.

In some embodiments the methods further comprise administering the engineered T cells to a subject in need thereof.

“Preventing T cell exhaustion” refers to a condition of maintained or restored functionality of T cells characterized by one or more of the following compared to cells in an exhausted state: decreased expression and/or level of one or more of PD-1, TIM-3, and LAG-3; increased memory cell formation and/or maintenance of memory markers (e.g., CD62L); prevention of apoptosis; increased antigen-induced cytokine (e.g., IL-2) production and/or secretion; enhanced killing capacity; increased recognition of tumor targets with low surface antigen; enhanced proliferation in response to antigen; and lower expression of inhibitory receptors (e.g., programed cell death 1 (PDCD1, also called PD1) and cytotoxic T lymphocyte-associated Antigen 4 (CTLA-4)).

Accordingly, the engineered T cells may display increased functionality and/or activity (e.g., increased antigen induced cytokine production, enhanced killing capacity (e.g., increased recognition of tumor targets with low surface antigen), increased memory cell formation, and/or enhanced proliferation in response to antigen) and/or reduced features of exhaustion (e.g., lower levels of markers or inhibitory receptors indicative of exhaustion (e.g., PD-1, TIM-3, LAG-3) and/or lower levels of programmed cell death) compared to non-modified T cells. In the context of therapeutic applications, the modified T cells may enhance the clinical efficacy of the therapeutics (e.g., CAR T cells).

In some embodiments, the isolated T cells further comprise a nucleic acid encoding an exogenous receptor. In some embodiments, the exogenous receptor is a T cell receptor (TCR) or a chimeric antigen receptor (CAR). Descriptions of methods for modifying the T cell, the exogenous receptor and the nucleic acids and target antigens thereof, the subject, and the disease and disorders set forth above in connection with the disclosed T cells, and compositions and methods thereof are also applicable to the method of preventing exhaustion of engineered T cells.

An effective amount of the modified T cells or compositions disclosed herein may be determined based on the type of disease to be treated, the type of modified T cell, the severity and course of the disease, the clinical condition of the individual, the individual's clinical history and response to the treatment, and the discretion of the attending physician.

The efficacy of any of the methods described herein (e.g., treatment of disease or disorder) may be tested in various models known in the art, such as clinical or pre-clinical models. Effectiveness of the treatment may refer to any one or more of: extending survival (including overall survival and progression free survival); resulting in an objective response (including a complete response or a partial response); or improving signs or symptoms of the disease or disorder (e.g., cancer or an infection disease).

In some embodiments, a sample is obtained prior to treatment with T cells (e.g., alone or in combination with another therapy described herein) as a baseline for measuring response to treatment. In some embodiments, the sample is a tissue sample (e.g., formalin-fixed and paraffin-embedded (FFPE), archival, fresh, or frozen). In some embodiments, the sample is whole blood. In some embodiments, the whole blood comprises immune cells, circulating tumor cells and any combinations thereof.

For any exemplary cancer model, after developing tumors, mice may be placed into treatment groups receiving treatment or control treatment. Tumor size (e.g., tumor volume) is measured during the course of treatment, and overall survival rate is also monitored.

In some embodiments, efficacy may refer to improvement of one or more factors according to the published set of RECIST guidelines for determining the status of a tumor in a cancer patient, e.g., responding, stabilizing, or progressing. A responsive subject may refer to a subject whose cancer(s) show improvement, e.g., according to one or more factors based on RECIST criteria. A non-responsive subject may refer to a subject whose cancer(s) do not show improvement, e.g., according to one or more factors based on RECIST criteria.

Effectiveness may also refer to improvement of one of more immune-related response criteria (irRC). In some embodiments, new lesions are added into the defined tumor burden and followed, e.g., for radiological progression at a subsequent assessment. In some embodiments, the presence of non-target lesions is included in assessment of complete response and not included in assessment of radiological progression. In some embodiments, radiological progression may be determined only on the basis of measurable disease and/or may be confirmed by a consecutive assessment following a period of time (e.g., four weeks) from the date first documentation.

### 6. Methods of Screening for T Cell Exhaustion Genes

The present disclosure also provides for screening for genes which facilitate T cell exhaustion. The methods comprise: culturing T cells under conditions of chronic or acute stimulation for at least six days, wherein the T cell comprises at least one gene knockout or knockdown; isolating T cells not showing an exhausted T cell surface phenotype; and identifying the at least one gene knockout or knockdown. In some embodiments, the T cells are a T cell library, wherein the T cell library comprises at least one T cell for each gene in the genome of the T cell.

The T cells may be selected from CD3+ T cells (e.g., a combination of CD4+ and CD8+ T cells), CD8+ T cells, CD4+ T cells, natural killer (NK) T cells, alpha beta T cells, gamma delta T cells, or any combination thereof. In some embodiments, the T cells are memory T cells (e.g., central memory T cells or effector memory T cells). In some embodiments, the T cells are tumor infiltrating lymphocytes. In some embodiments, the T cells are cytokine-induced killer cells. In select embodiments, the T cells are CD8+ T cells.

In some embodiments, the T cells are naturally occurring T cells. For example, the T cells may be isolated from a subject sample.

The T cells or T cell library may be generated using methods known in the art for genetic screening, e.g., RNAi, complementary DNA (cDNA) libraries, or CRISPR/Cas9-based genome editing. The method may be designed to measure single gene knockdowns or knockouts separately. Alternatively, the method may be designed to measure combinatorial gene knockdowns or knockouts.

In some embodiments, the T cells are generated by performing single or combinatorial CRISPR-Cas-based gene knockdowns with a genome-wide library of guide RNAs. Thus, in certain embodiments, the T cells are generated using a CRISPR-Cas system wherein each cell comprises at least one guide RNA. See for example, U.S. Patent Application 20190085324, incorporated herein by reference in its entirety.

CRISPR-Cas systems, e.g., CRISPR-Cas9 systems, as used herein, refer to non-naturally occurring systems derived from bacterial Clustered Regularly Interspaced Short Palindromic Repeats loci. These systems generally comprise an enzyme (Cas protein, such as Cas9 protein) and one or more guide RNAs. The CRISPR-Cas system may be engineered, for example for optimal use in mammalian cells, for optimal delivery therein, for optimal activity in gene editing.

The guide RNA (gRNA) may be a crRNA, crRNA/tracrRNA (or single guide RNA, sgRNA). The terms “gRNA,” “guide RNA” and “CRISPR guide sequence” may be used interchangeably throughout and refer to a nucleic acid comprising a sequence that determines the binding specificity of the CRISPR-Cas system. A gRNA hybridizes to (complementary to, partially or completely) a target nucleic acid sequence (e.g., a gene in the genome of a cell).

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. Jan. 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

For genome-wide approaches, it is possible to design and construct suitable gRNA libraries. Such gRNAs may be delivered to cells using vector delivery such as viral vector delivery. Combination of CRISPR-Cas-mediated perturbations may be obtained by delivering multiple gRNAs within a single cell.

T cells cultured under conditions of chronic or acute stimulation may become exhausted. As described herein, an exhausted T cell surface phenotype comprises increased concentrations of PD-1, TIM-3, and LAG-3. Thus, any stimulation conditions which result in T cell exhaustion may be used in the disclosed methods. In some embodiments, conditions of chronic stimulation comprise culturing the T cells using anti-CD3 coated plates. In some embodiments, the chronic stimulation conditions further comprise culturing in the presence of IL-2. In some embodiments, conditions of acute stimulation comprise culturing the T cells in the presence of IL-2.

The culturing may last for any period of time necessary for onset of T cell exhaustion. In some embodiments, the culturing is at least 6 days. In some embodiments, the duration of the culturing is for 6-10 days (e.g., 6 days, 7 days, 8 days, 9 days, or 10 days). In some embodiments, the culturing lasts for more than 10 days.

The T cells may exist in culture prior to the culturing under conditions of chronic or acute stimulation. For example, the T cells may be cultured under normal culture conditions for growth, reproduction, or genetic engineering prior to placing the T cells under conditions of chronic or acute stimulation.

Isolating T cells not showing an exhausted T cell surface phenotype includes any method(s) which allow identification of exhausted T cells and/or separation of identified cells. For example, FACS analysis of markers of T cell exhaustion, as described elsewhere herein, enables identification and removal of non-exhausted T cells prior to identification of the at least one gene knockout or knockdown.

To identify the genes which facilitate T cell exhaustion, those T cells which do not exhibit an exhausted phenotype are isolated and the genomic DNA is extracted for analysis. The analysis may comprise sequencing of the genome to determine the knocked out gene. In the case of a CRISPR-Cas screen, the gRNA-encoding regions are subjected to PCR amplification, sequenced, and mapped to the gRNA library. By comparing the gRNA profiles, the link between the knockout and T cell exhaustion can be determined.

The disclosure further provides systems or kits containing one or more reagents or other components useful, necessary, or sufficient for practicing any of the methods described herein. The systems or kits may include exogenous receptor reagents (nucleic acids, vectors, compositions, etc.), transfection or administration reagents, negative and positive control samples (e.g., T cells or empty vector DNA), T cells, system for genetic engineering T cells (e.g., Cas proteins, gRNAs, vectors thereof, etc.), additional therapeutic agents, containers (e.g., microcentrifuge tubes), detection and analysis instruments, software, instructions, and the like. Descriptions of nucleic acids, vectors, compositions, T cells, additional therapeutic agents provided elsewhere herein are suitable for use with the disclosed systems or kits.

In some embodiments, the systems or kits comprise engineered T cells as described herein or a system for genetic engineering T cells. The system for genetic engineering T cells may comprise a clustered interspersed short palindromic repeat (CRISPR)/CRISPR-associated protein (Cas) system, as described herein. In certain embodiments, the system for genetic engineering T cells comprises a Cas protein (e.g., Cas9, dCas9), or a nucleic acid encoding a Cas protein, and a gRNA directed to at least one gene which facilitates T cell exhaustion, or a nucleic acid encoding the gRNA. In some embodiments, the nucleic acid encoding the Cas protein (e.g., Cas 9) and the gRNA are the same or different nucleic acid. For example, the gRNA and the Cas protein may be expressed from the same vector. The at least one gene which facilitates T cell exhaustion may be selected from the group consisting of: INO80C, GATA3, ARID1A, WDR82, TRP53, GPR137C, ZFP219, HDAC1, ELMSAN1, and ACTR8. The at least one gene which facilitates T cell exhaustion may encode a chromatin remodeling protein. In some embodiments, the chromatin remodeling protein is a INO80 nucleosome positioning complex protein or SWI/SNF family member, or a combination thereof. In some embodiments, the INO80 nucleosome positioning complex protein is Actr5, Ino80, Ino80c, Ino80b, Actr8, or a combination thereof. In some embodiments, the SWI/SNF family member is a member of cBAF complex. In some embodiments, the SWI/SNF family member is Arid1a, Arid2, Arid1b, Smarcb1, Smarcd2, Smarca4, Smarcc1, or a combination thereof.

In some embodiments, the systems or kits further comprise an exogenous receptor or a nucleic acid encoding thereof.

In some embodiments, the systems or kits further comprise at least one additional therapeutic agent. The at least one therapeutic agent may be selected from the group consisting of: an agent for treating T cell exhaustion; an antiviral agent; an antibiotic agent; an antimicrobial agent; a chemotherapeutic agent; or a combination thereof.

In some embodiments, the systems or kits further comprise instructions for using the components of the system or kit. The instructions are relevant materials or methodologies pertaining to the systems or kits. The materials may include any combination of the following: background information, list of components and their availability information (purchase information, etc.), brief or detailed protocols for using the systems or kits, trouble-shooting, references, technical support, and any other related documents. Instructions can be supplied with the systems or kits or as a separate member component, either as a paper form or an electronic form which may be supplied on computer readable memory device or downloaded from an internet website, or as recorded presentation.

### 7. Examples

### Materials and Methods

Mice All mice were procured from JAX. Wild type mice were C57BL/6J mice (JAX: 000664). Cas9 knock-in mice were bred in house (JAX: 026179). OT-1 mice (JAX: 003831) were crossed with Cas9 mice. Rag1^{-/-} mice were bred in house (JAX: 002216).

Primary T cell isolation and culture Spleens were collected and mashed through a 70 uM filter. RBCs were lysed with ACK lysis buffer (Gibco) and incubated for 6 mins before washing with PBS. Cells were counted and then resuspended in MACS buffer (PBS+0.5% BSA+2 μM EDTA) according to Miltenyi protocol. CD8 T cells were enriched using the mouse CD8 T cells isolation kit from Miltenyi and then resuspended in RPMI with 10% FBS, 1% Sodium pyruvate, 1% Non-essential amino-acids, 100 U Pen/Strep, 50 nM of B-mercaptoethanol (cRPMI) and supplemented with 10 ng/ml of mouse IL-2. Cells were seeded at a concentration of 1 million cells/ml on plates coated with 5 ug/ml of anti-CD3 and 2 ug/ml of anti-CD28. Cells were kept on these activation plates for 48 hours at the beginning of all experiments. CD8′ T cell purity was verified via flow cytometry. Cells were passaged every two days and maintained at 1 million cells per mL.

In vitro T cell exhaustion assay To induce T cell exhaustion, chronic stimulation was performed using anti-CD3 coated plates at 5 ug/mL (in the continued presence of 10 ng/ml IL-2). Cells were passaged onto a fresh coated plate every two days and analyzed on Day 6, 8, or 10 as described in the Results. In contrast, acutely stimulated cells were maintained in 10 ng/ml IL-2 alone, passaged every two days, and analyzed on Day 6, 8, or 10

Measurement of cytokine production T cells were re-stimulated with phorbol myristate acetate (Sigma, 50 ng ml⁻¹) and ionomycin (Sigma, 500 ng ml⁻¹) or plate bound anti-CD3 at 3 μg/ml. After 90 min, cells were treated with brefeldin A to block cytokine secretion. Then, 3 h later, cells were stained for surface markers and simultaneously labeled with Live/Dead Blue Viability Dye (Thermo Fisher) for 20 min at 4° C. Cells were washed twice and fixed overnight using a FoxP3 Fixation/Permeabilization Kit (Thermo Fisher). The following day, cells were washed and stained for intracellular cytokines at room temperature for 1 h. They were then washed three times and analyzed using an LSR Fortessa machine (Beckman Dickinson). Analysis of mean fluorescence intensity was performed using FlowJo v.10.0. All experiments were performed at least two biological replicates. Antibodies used (at 1:100 unless otherwise noted) were TNF-PE (BioLegend, MP6-XT22, 506306), PD-1-PECy7 (BioLegend, RMP1-30, 109110) IFN-γ-FITC (BioLegend, XMG1.2, 505806), CD4-BV711 (BioLegend, RM4-5, 100550), and CD8α-BV786 (BioLegend, 53-6.7, 100750).

Growth curves T cells were activated, as described. They were subsequently plated in 24-well plates at 5×10⁵ cells in 1 ml of RPMI-1640 medium containing 10% FBS, 2 mM 1-glutamine, 5 μM β-ME and 10 ng ml⁻¹ IL-2, and with (chronic) or without (acute) plate-bound anti-CD3 (3 μg ml⁻¹). Every 2 d for the duration of the experiment, cells were collected, and cell number was counted using a Beckmann Coulter Counter with a cell volume gate of 75-4,000 femtoliters. Then, 50% of the cells were re-plated in 1 ml of fresh T cell medium. All experiments were performed at least two independent times.

In vitro killing assay B16 cells expressing a Luciferase reporter were pulsed with SIINFEKL peptide (Invivogen) at the concentrations noted for 4 h at 37° C. They were then washed twice and plated at 4×10⁴ cells per well along with 1×10⁵ OT-1 transgenic T cells that had been acutely or chronically stimulated for 8 days as previously described. After 24 h of co-culture, cells were lysed and luciferase activity was measured using a Luciferase Assay Kit (Promega) as per manufacturer's instructions. Luciferase activity was normalized to cells cultured in the absence of T cells

B16-ovalbumin in vivo tumor models C57BL/6 scid (Jackson 001913) mice were injected subcutaneously with 2×10⁵ B16-OVA cells in a 1:1 mix of PBS and Matrigel (Corning). At 5 d later, 2×10⁶ OT-1 T cells that had been acutely or chronically stimulated as described previously were adoptively transferred to mice via retro-orbital injection. Mice were monitored daily and were killed for signs of morbidity.

ATAC-seq sample processing and analysis ATAC-seq was performed using the Omni-ATAC protocol (Corces et al., 2017, Nat. Methods 14, 959-962). Briefly, 50,000 live cells were purified by flow cytometry immediately prior to ATAC-seq. Lysis, nuclei isolation, and transposition were performed according to the Omni-ATAC protocol. Libraries were prepared for sequencing and sequenced in 2×75 dual-indexed format on an illumina NovaSeq.

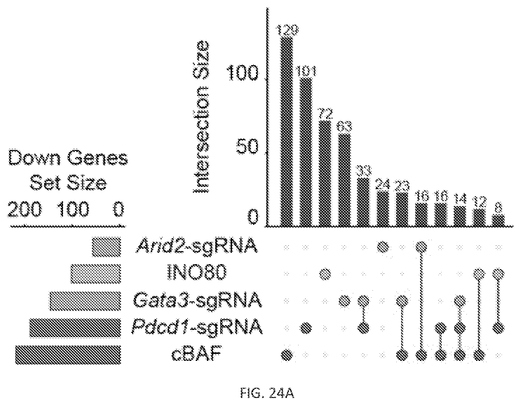

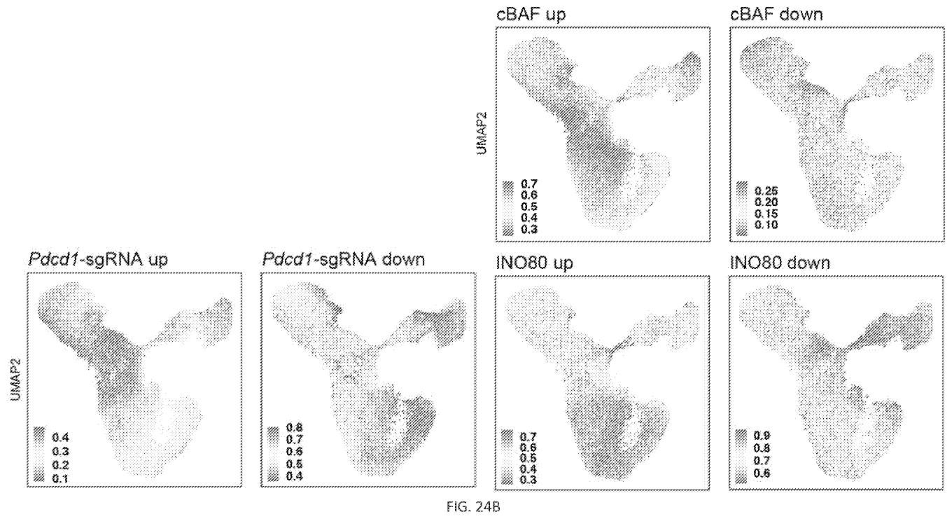

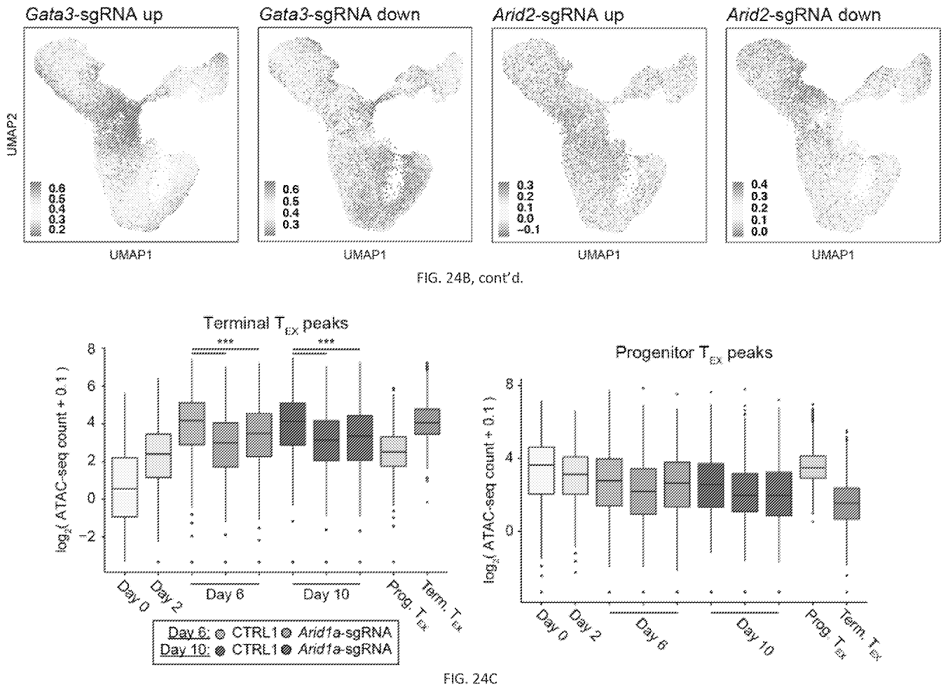

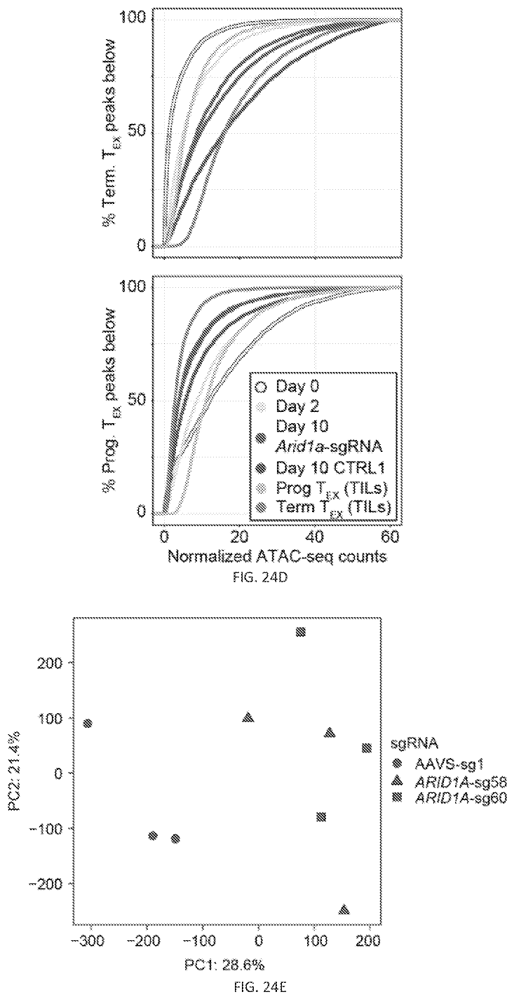

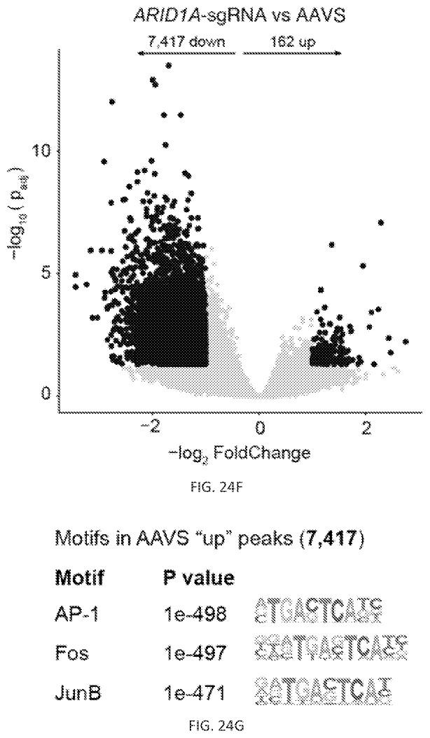

Fastq files were trimmed using fastp and aligned to the mm10 genome using hisat2. Reads were deduplicated and a bed file for each sample containing filtered, deduplicated ATAC-seq fragments was created. Peaks for each sample were called individually using MACS2 and then filtered into reproducible peaks based on peaks present in the majority of replicates for that sample. A union peak set for all samples was constructed by merging reproducible peaks for each sample into a set of high-confidence non-overlapping fixed width (500 bp) peaks, which was used to create a peak by sample matrix used in downstream analysis. Differential peaks were determined using DESeq2 (Love et al., 2014). Principal component analysis was performed on the peak matrix by first normalizing using ‘DESeq2::varianceStabilizingTransformation’ and then ‘stats::prcomp’. Genome track files were created by loading the fragments for each sample into R, and exporting bigwig files normalized by reads in transcription start sites using ‘rtracklayer::export’. Coverage files were visualized using the Integrative Genomics Viewer. For analysis of previously published ATAC-seq data (Miller et al., 2019, Nat. Immunol. 20, 326-336), fastq files were downloaded from accession GSE123236 and re-processed using the disclosed pipeline for consistency. Terminal and Progenitor T_{EX} ATAC-seq peaks were computed using DESeq2 with cutoffs of Log₂ FC≥1 and FDR≤0.05 when comparing Terminal versus Progenitor T_{EX} samples (either TIL samples or LCMV samples, as indicated). For quantification of overlapping peaks between published data and in vitro assay data, a union peak set was created encompassing all samples and re-analyzed. For HOMER motif enrichment analysis, as shown in FIG. 24G, the HOMER findMotifsGenome command line utility was used to identify motifs present in peaks in the indicated peak set relative to a background peak set. For the background peak set, the union peak set of the considered samples was used, and as a result the enriched motifs correspond to motifs enriched in the differential peak set relative to samples in aggregate, rather than motifs enriched in human T cells relative to random genomic regions.

Genome-wide sgRNA library Retroviral Mouse Genome-wide CRISPR Knockout Library was a gift from Sarah Teichmann (Addgene #104861). The library was amplified via electroporation and confirmed by sequencing.

sgRNA Pool Design and Cloning

sgRNA mini-pool was designed using a previously developed protocol for cloning into a lentiviral backbone and then subcloned into retroviral construct pMSCV (Flynn et al., 2021, Cell 184, 2394-2411). lentiCRISPR-v2 was a gift from Feng Zhang (Addgene plasmid #52961). pMSCV-U6sgRNA(BbsI)-PGKpuro2ABFP was a gift from Sarah Teichmann (Addgene plasmid #102796).

Briefly, six 20 bp variable sgRNA sequences per target gene were obtained from the Broad Genetic Perturbation Platform (GPP) genome wide designs: sgRNA_design_10090_GRCm38_SpyoCas9_CRISPRko_NCBI_20200317(dot)txt(dot)gz, available online at portals(dot)broadinstitute(dot)org/gpp/public/dir?dirpath=sgrna_design. One hundred non-targeting and 100 single-targeting negative control guides designed for the mouse genome, also from the Broad GPP web portal, were included. A “G” was added to the start of each 20 bp sequence. This 21 bp sequence was flanked by BsmBI-v2 enzyme sites and then two nested PCR handles. Pooled oligos were synthesized by Twist Bioscience. Oligos were amplified by two rounds of PCR and the lentiCRISPR-v2 backbone was digested overnight with Esp3I. One step digestion/ligation of amplified oligos into lentiCRISPR-v2 was performed at 37° C. for 1 hour in a 20 uL reaction with 1 uL T4 ligase, 1 uL Esp3I, 2 uL T4 ligase buffer, 200 ng digested backbone, and 50 ng amplified insert. Reaction was heat inactivated for 15 minutes at 65° C. and then 1 uL was electroporated using 25 uL Lucigen Endura electrocompetent cells and a BioRad MicroPulser with 0.1 cm gap cuvettes. After 1 hour recovery in SOC, a 1000× dilution was plated onto an agar plate to confirm library coverage. The remainder was cultured overnight in a 150 mL liquid culture and then purified by maxiprep. Finally, the pool was subcloned into pMSCV by Gibson Assembly of the sgRNA variable region amplified via PCR and pMSCV backbone pre-digested with BbsI. Electroporation was repeated as described above. Guide representation was confirmed by sequencing.

The sgRNA SWI/SNF mini-pool and micro-pool for perturb-seq were designed with 4 guides per gene, as described above for the mini-pool using the Broad GPP mouse genome-wide designs. The SWI/SNF mini-pool contained 50 single-targeting controls and Perturb-seq micro-pool contained 12 single-targeting controls. Two primers were ordered per designed guide, for cloning via annealing. The pMSCV vector was digested with BbsI. All primer pairs were annealed separately. Annealed products were pooled equally, diluted, and then ligated into pMSCV. Amplification was performed using Stbl3 Chemically Competent cells (ThermoFisher C737303) and library coverage was confirmed via colony counting and then sequencing.

Retrovirus production and transduction The pMSCV plasmid was transfected into GP2-293 cells (Takara, RetroPack™ PT67 Cell Line) or 293T HEK cells at roughly 80% confluency in 15 cm tissue culture plates coated with poly-d-lysine. Viral supernatant was collected at 48 h and 72 h post-transfection, filtered via a 0.45 μm filtration unit (Millipore). Filtered virus was concentrated using the LentiX concentrator (Takara) at 1500×g for 45 minutes. The concentrated supernatant was subsequently aliquoted, flash frozen, and stored in −80° C. until use.

CD8 T cells were transduced with concentrated retrovirus 24 hours after isolation. 4 ug/ml of Polybrene was added to each well. Plates were sealed and then spun at 1100×g at 32° C. for 90 minutes. 24 hours after spinfection (e.g., starting on day 2) cells were checked for fluorescence via flow cytometry and 2 ug/mL puromycin was added to the media.

sgRNA library preparation and sequencing For samples from in vitro chronic culture, live cells were first isolated via FACS. gDNA was extracted using a commercially available Zymo kit. sgRNA libraries were prepared for sequencing as previously described (Flynn et al., 2021, Cell 184, 2394-2411). Briefly, a standard three-step amplification protocol was used. First, sgRNAs were amplified off of gDNA using primers specific to the pMSCV vector for 22 cycles of PCR. 100 uL reactions with up to 4 ug of gDNA per reaction were used, and the number of reactions was scaled up until all gDNA was used. For sequencing of plasmid pools, this first PCR was skipped. For the second PCR, a 0-7 bp offset was added to the front of the library using 8 pooled stagger primers to increase the diversity of the library. PCR2 primer target sites were nested inside those of PCR1 to improve the specificity of the product. Finally, in PCR3, index sequences were added. Libraries were sequenced in dual-indexed 1×75 bp or 1×150 bp format on either an Illumina NextSeq or NovaSeq.

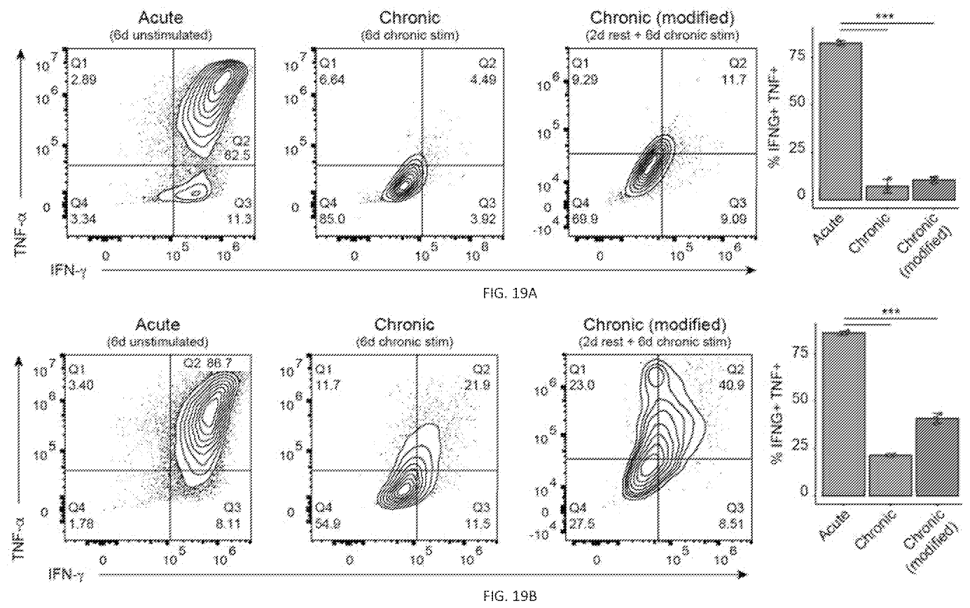

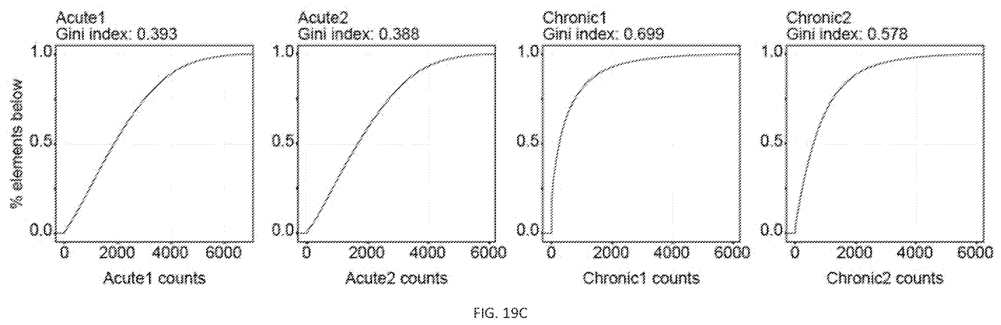

Bulk sgRNA screening data analysis sgRNA sequencing data was analyzed using previously published pipelines (Flynn et al., 2021, Cell 184, 2394-2411). Briefly, fastq files were trimmed using ‘fastp -f 10 --max_len1=50’. Trimmed reads were aligned to a custom fasta file of the relevant pool (either the genome wide pool or the minipool) which was constructed by taking the sgRNA variable sequences and flanking them with the adjacent sequences in the pMSCV vector backbone. Alignment was performed using hisat2 with the --no-spliced-alignment option. Bam files were imported into R and converted into counts per guide using ‘Rsamtools::scanBam’. A table of guides per sample was constructed in R and normalized by multiplying each count by 1e6, dividing by the total counts in that sample, adding 1, and then log 2 normalizing. Log fold changes between two conditions (e.g., chronic vs acute or tumor vs input) were computed and then z-scored by subtracting the reference LFC average and dividing by the reference LFC standard deviation. For genome-wide screens, all guides were used as the reference (e.g., guides were z-scored relative to all other guides) and for minipool screens the control guides were used as the reference. P-values were computed from z-scores using the normal distribution and then FDR was computed by correcting for multiple hypothesis testing using ‘p.adjust’ in R. For the Gini index analysis, as shown in FIG. 19, the ‘ineq’ R package was used.

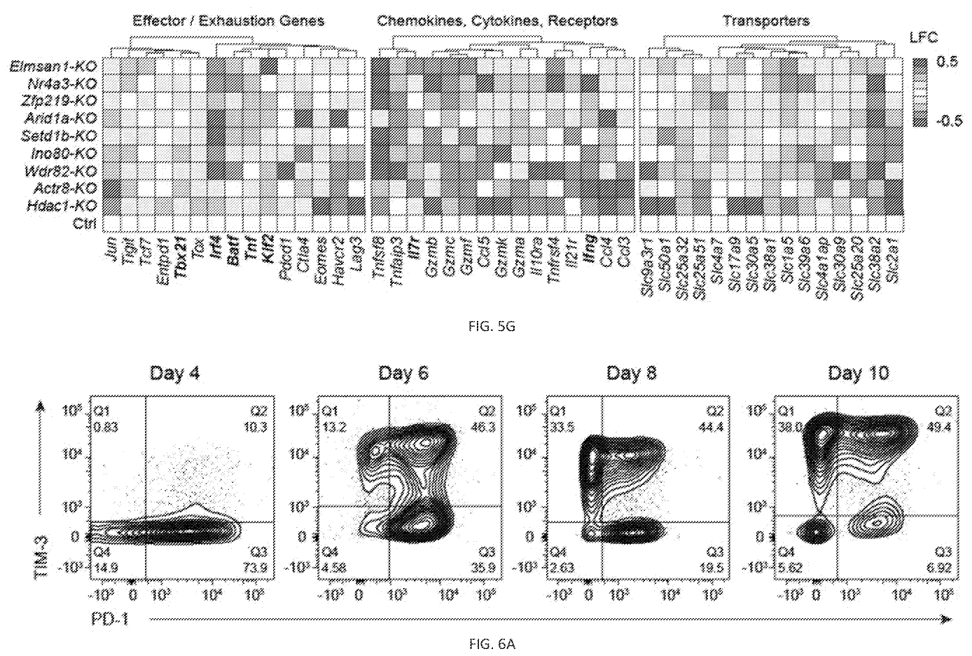

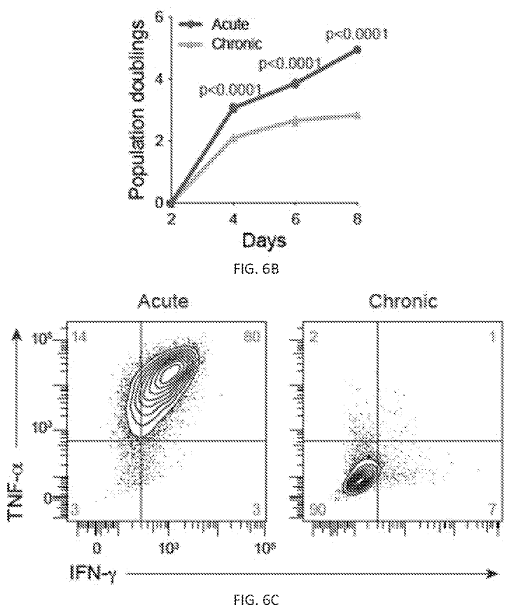

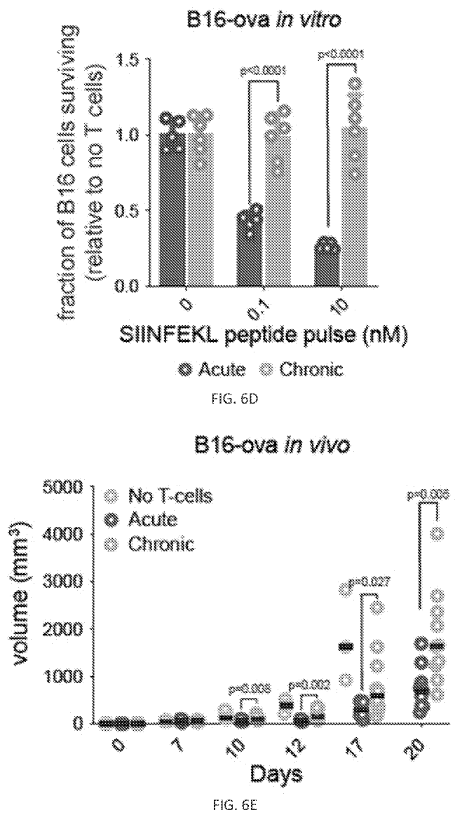

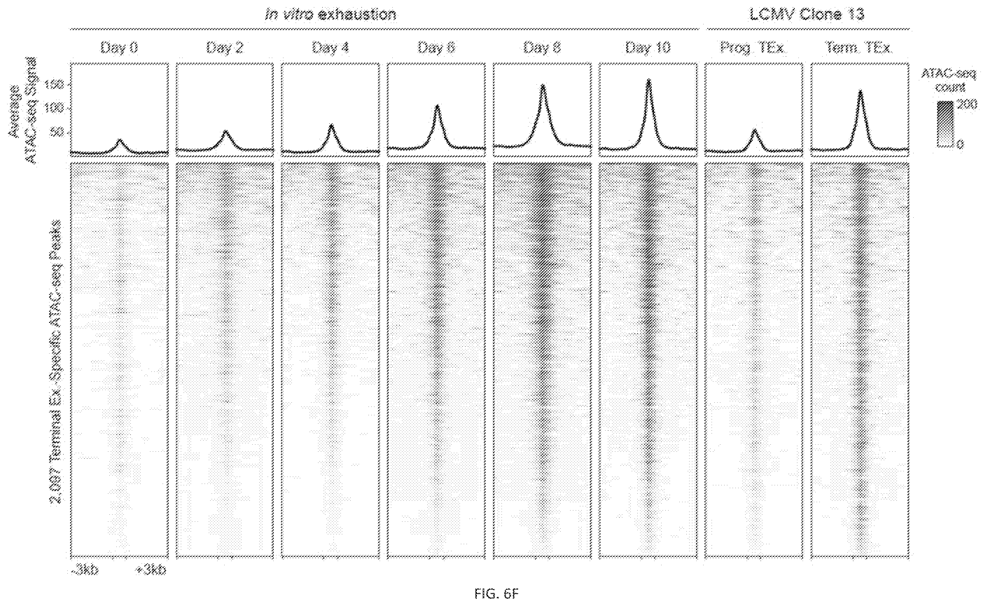

GO Term analysis For gene categorizations shown in FIG. 6C and elsewhere, gene sets were defined as: TCR—KEGG_T_CELL_RECEPTOR_SIGNALING_PATHWAY, Chromatin—GOCC_CHROMATIN, Integrin—GOBP_INTEGRIN_ACTIVATION, Inhibitory receptor—GOBP_NEGATIVE_REGULATION_OF_LYMPHOCYTE_ACTIVATION. Gene lists were manually supplemented with the following genes: Chromatin—ZFP219, TBX21, KDM6A, ELMSAN1, DNTTIP1, SETD1B, TADA2B, ZFP217, EOMES. Integrins—ITGB3, APBB1IP, ITGAV. Inhibitory receptors—PDCD1. For the gene set enrichment analysis, the indicated gene list was uploaded to the online gProfiler tool (available at biit(dot)cs(dot)ut(dot)ee/gprofiler/gost).

Cytoscape interaction network The top one hundred positive hits and top twenty negative hits were imported into Cytoscape. Edges were created by using the stringApp Cytoscape plugin to import known protein-protein interactions curated from string-db (Szklarczyk et al., 2019, Nucleic Acids Res. 47, D607-D613). A cutoff of stringdb score≥0.75 was used to filter these protein-protein interactions, which represents a conservative cutoff for identifying only high confidence interactions. Nodes were grouped based on GO Term analysis, subcellular localization, and/or manual curation. A small number of poorly characterized and/or disconnected nodes were removed from the visualization.

Tumor inoculation and T cell adoptive transfer for in vivo CRISPR experiments MC-38 of B16 cells ectopically expressing an mCherry-ovalbumin fusion construct were prepared for injection by resuspending in a 1:1 mixture of matrigel and PBS. 1×10⁶ cells per tumor were injected subcutaneously into the flanks of Rag1^{-/-} mice (two tumors per mouse). Tumors were measured every three days. Cas9-OT-1 CD8+ T cells were transduced with sgRNA pools or individual sgRNAs and selected with puromycin for 4 days, as described above. T cells were then intravenously injected into tumor-bearing mice. For in vivo competition assays, cells were mixed immediately prior to injection. Nine days after T cell injection, the spleen and tumors were harvested from each mouse.

Tissue Processing and Isolation of Tumor Infiltrating Lymphocytes Tumors were weighed and then minced into small pieces. The tumors were transferred to a gentleMACS C tube and digested in the protocol recommended enzyme mix with a gentleMACS octo dissociator using the listed soft/medium tumor program. Tumor suspensions were then filtered with a 70 uM filter and then subject to RBC lysis. Spleens were mashed and filtered through a 70 uM strainer, then treated with RBC lysis buffer. For bulk sgRNA sequencing and perturb-seq, tumor infiltrating lymphocytes or T cells were isolated from the tumors or spleens by FACS. Samples were washed twice with MACS buffer and stained for 30 mins on ice. CD8+ BFP+ cells were isolated via flow cytometry.

Competition assay for validation of individual sgRNA proliferation The pMSCV retroviral vector was modified to replace the BFP-puromycin fusion with a VEX-puromycin fusion. Individual guides were cloned by annealing pairs of primers, as described above. The Arid1a-1 sgRNA sequence used was GCAGCTGCGAAGATATCGGG (SEQ ID NO: 2) and the Arid1a-2 sequence used was CAGCAGAACTCGCACGACCA (SEQ ID NO: 3). The CTRL sgRNA sequence used was CTTACTCGACGAATGAGCCC (SEQ ID NO: 4). Tumor processing was performed as described above for the in vivo validation.

Validation of Arid1a-targeting sgRNAs Tracking of indels by decomposition (TIDE): Genomic DNA was isolated from transduced cells using a commercially available kit (Zymo Cat #D3025). PCR reactions were performed with primers surrounding the expected edit site and 50 ng of input DNA. PCR conditions were 30 seconds at 980 C, followed by 10 seconds at 980 C, 10 seconds annealing at 60° C., 25 seconds at 72° C. for 35 cycles, then 2 minutes at 72° C. The PCR amplicons were purified with a commercially available Zymo DNA clean up kit and sanger sequenced. Quantification of edits was performed using the online tool tide(dot)nki(dot)nl.

Western blot: Protein lysates were prepared from mouse T cells transduced with the indicated sgRNA using a radioimmunoprecipitation assay (RIPA) buffer system (Santa Cruz, sc-24948). Protein concentrations were quantified using the bicinchoninic Acid (BCA) assay (Pierce, ThermoFisher 23225). 20 μg of protein per sample was loaded and run on a 4-12% Bis-Tris PAGE gel (NuPAGE 4-12% Bis-Tris Protein Gel, Invitrogen) and transferred onto a polyvinylidene fluoride (PVDF) membrane (Immobilon-FL, EMD Millipore). Membranes were blocked with 5% milk in PBST for 1 h at room temperature (RT) and incubated with primary antibodies against Arid1a (rabbit, 1:1000, Cell Signaling, 12354S: Lot 4), Arid1b (mouse, 1:1000, Abcam, ab57461: Lot GR3345290-4), Smarca4 (rabbit, 1:1000, Cell Signaling, 49360S: Lot 3) and Tbp (mouse, Abcam, ab51841: Lot GR3313213-3) overnight at 4° C. Membranes were washed three times with PBST and then incubated with near-infrared fluorophore-conjugated species-specific secondary antibodies: Goat Anti-Mouse IgG Polyclonal Antibody (IRDye 680RD, 1:10,000, LI-COR Biosciences, 926-68070) or Goat Anti-Rabbit IgG Polyclonal Antibody (IRDye 800CW, 1:10,000, LI-COR Biosciences, 926-32211) for 1 hour at RT. Following secondary antibody application, membranes were washed three times with PBST, and then imaged using a LI-COR Odyssey CLx imaging system (LI-COR). Protein band intensities were quantified using Image Studio Lite (LI-COR) with built-in background correction and normalization to Tbp controls. Statistical analysis comparing Arid1a levels normalized to Tbp was performed using Dunnett's multiple comparisons test on Prism (v9.2.0).

In vitro experiments in primary human T cells T cell expansion and viability assays: T cells were activated for 4 days at a 1:3 ratio of T cells to anti-CD3/28 Dynabeads (Invitrogen). T cell expansion assays were performed with IL-2 in the culture medium at 10 ng/mL. Cell counts and viability measurements were obtained using the Cellaca Mx Automated Cell Counter (Nexcelom). Cells were stained with acridine orange and propidium iodide to assess viability.

Targeted CRISPR gene editing: Ribonucleoprotein (RNP) was preparing using synthetic sgRNA with 2′-O-methyl phosphorothioate modification (Synthego) diluted in TE buffer at 100 μM. 5 μl sgRNA was incubated with 2.5 μl Duplex Buffer (IDT) and 2.5 μg Alt-R S.p. Cas9 Nuclease V3 (IDT) for 30 minutes at room temperature. 100 μl reactions were assembled with 10 million T cells, 90 μl P3 buffer (Lonza), and 10 μl RNP. Cells were pulsed with protocol E0115 using the P3 Primary Cell 4D-Nucleofector Kit and 4D Nucleofector System (Lonza). Cells were recovered immediately with warm media for 6 hours. Guide sequences: AAVSI-sg1 5′ GGGGCCACUAGGGACAGGAU 3′ (SEQ ID NO: 5), ARID1A-sg58 5′ CCUGUUGACCAUACCCGCUG 3′ (SEQ ID NO: 6), ARID1A-sg60 5′ UGUGGCUGCUGCUGAUACGA 3′ (SEQ ID NO: 7).

Assessment of targeted CRISPR gene editing: 4-7 days after editing, genomic DNA was extracted with QuickExtract DNA Extraction Solution (Lucigen) and ˜500 bp regions flanking the cut site were amplified with Phusion Hot Start Flex 2X Master Mix (New England Biolabs) according to manufacturer's instructions. Sanger sequencing traces were analyzed by Inference of CRISPR Editing (ICE).

Pooled CRISPR screen in primary human T cells in vivo Activated human T cells from two donors were transduced by lentivirus to express the NY-ESO specific TCR, in parallel to lentiviral transduction of a sgRNA library with 2 sgRNAs per target gene and 8 negative controls. 24 hours after transduction, cells were electroporated with Cas9 Protein, as previously described (Shifrut et al., 2018, Cell 175, 1958-1971.e15). After electroporation, T cells were expanded in complete X-vivo 15 medium and split every two days, supplementing IL-2 at 50 U/ml. On Day 7, 2 NSG mice per donor were injected subcutaneously with 1×10⁶ A375 cells, as previously described (Roth et al., 2020, Cell 181, 728-744.e21). 1×10⁶ TCR-positive T cells were transferred to mice 7 days later via retro-orbital injection. Tumors and spleens were collected 7 days after T cell transfer and processed to single cell suspension, as described previously (Roth et al., 2020, Cell 181, 728-744.e21). T cells were sorted by CD45 staining and gDNA was extracted using commercial kits. Library preparation, next generation sequencing and analysis was performed as previously described (Shifrut et al., 2018, Cell 175, 1958-1971.e15). The guide abundance in the spleen and tumor of each mouse was used to calculate log fold change of each guide, and MAGeCK scores were calculated with defaulk parameters.

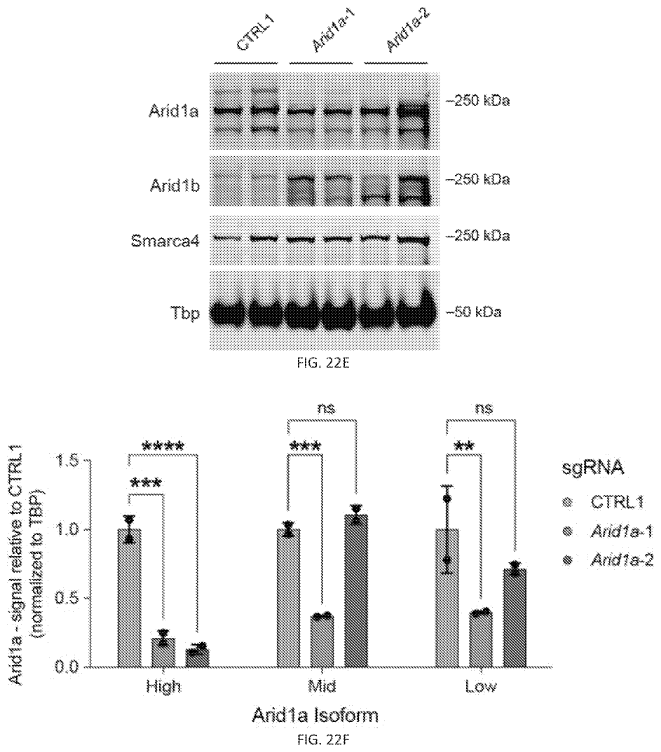

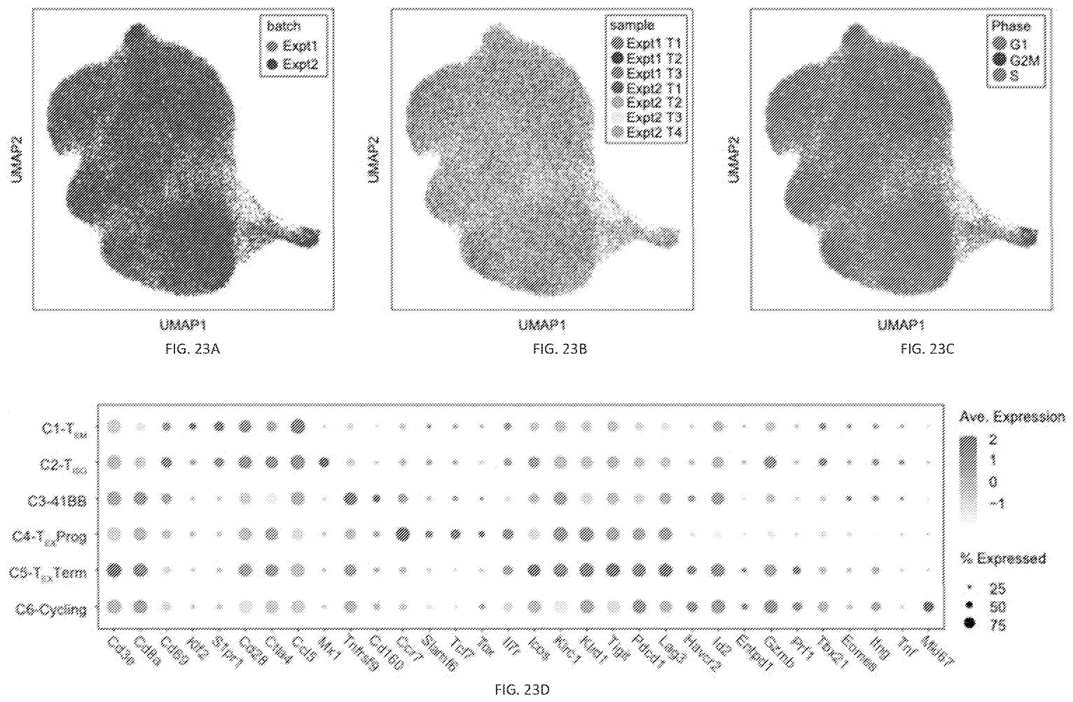

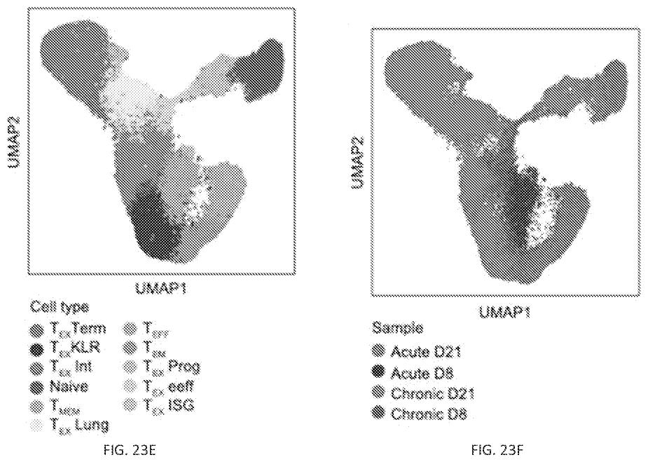

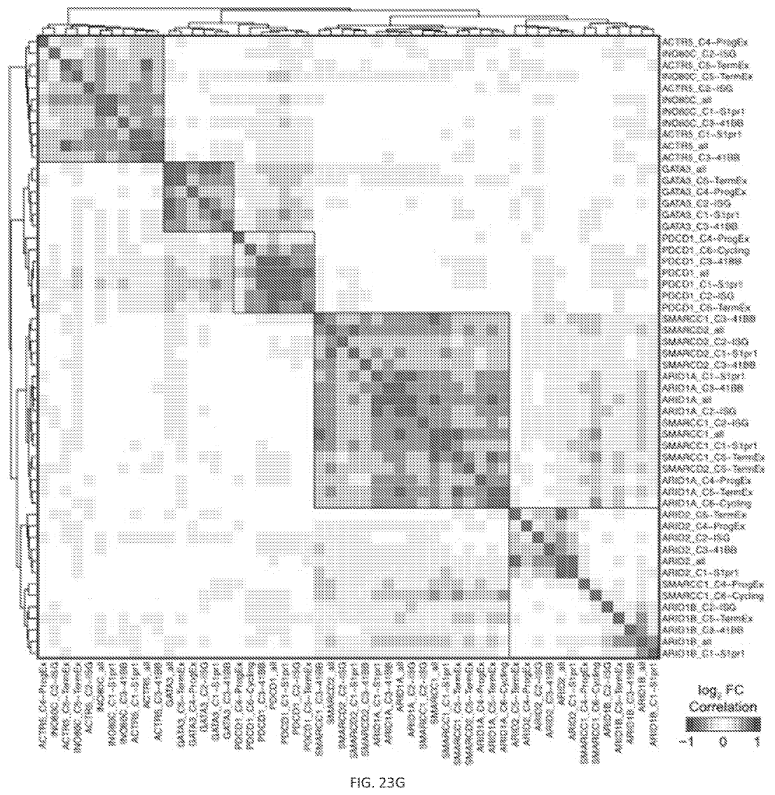

Direct capture Perturb-seq The 10× Chromium Next GEM Single Cell V(D)J Reagent Kits v1.1 5′ scRNA with Feature Barcoding reagents and protocol were adapted to be compatible with direct capture of sgRNAs in single cells. The modifications to the protocol are summarized here. For Step 1, GEM Generation and Barcoding, 5 pmol of primer KP_bead_sgRNA_RT was spiked into the reaction, enabling capture of sgRNAs in droplets and then reverse transcription of sgRNAs. Step 3.2B, Supernatant Cleanup for Cell Surface Protein Library was performed to isolate sgRNA library. Finally, 2 uL of the product of Step 3.2B was amplified and indexed using 3 rounds of PCR. The 250 bp library was purified via agarose gel and sequenced together with the gene expression (GEX) library in 26×91 format, according to 10× protocol guidelines. For Perturb-seq replicate samples shown in FIG. 23B, each replicate represents either an individual tumor or two tumors from the same mouse combined into one sample. Tumors from the same mouse were combined if the cell yield was well under 10× guidelines for targeted recovery of 10,000 cells per capture. If cell yield was well over the amount needed for recovery of 10,000 cells, in certain cases samples were split across multiple 10× captures to maximize cell yield. Samples split across multiple captures were computationally merged and not counted as separate replicates.

Fastq files were processed using the 10× cellranger count pipeline with feature barcode analysis enabled to process the GEX library and sgRNA library together. The mm10 reference transcriptome was used for the GEX library. For the sgRNA library, a feature reference spreadsheet was constructed which contained the variable sequence of each guide (reverse complemented since it was sequenced as part of read 2), guide ID, and target gene. The filtered matrices for both ‘Gene Expression’ and ‘CRISPR Guide Capture’ were loaded into Seurat for downstream analysis (Hao et al., 2021, Cell 184, 3573-3587). The Seurat ‘IntegrateData’ utility was used to merge the samples from the two independent experiments.

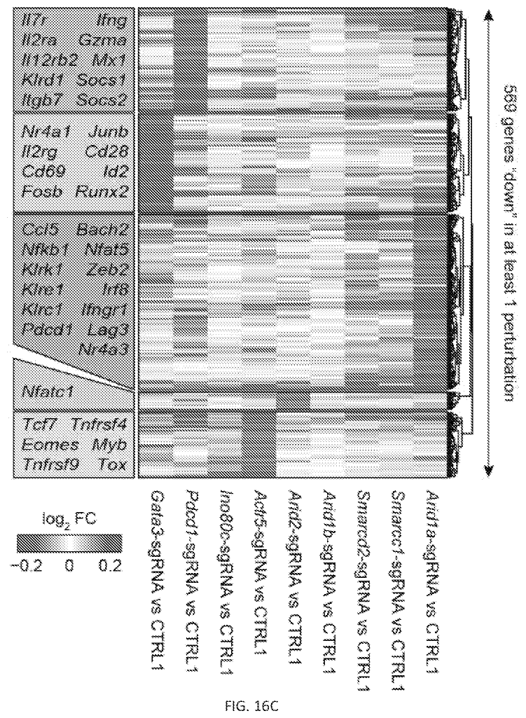

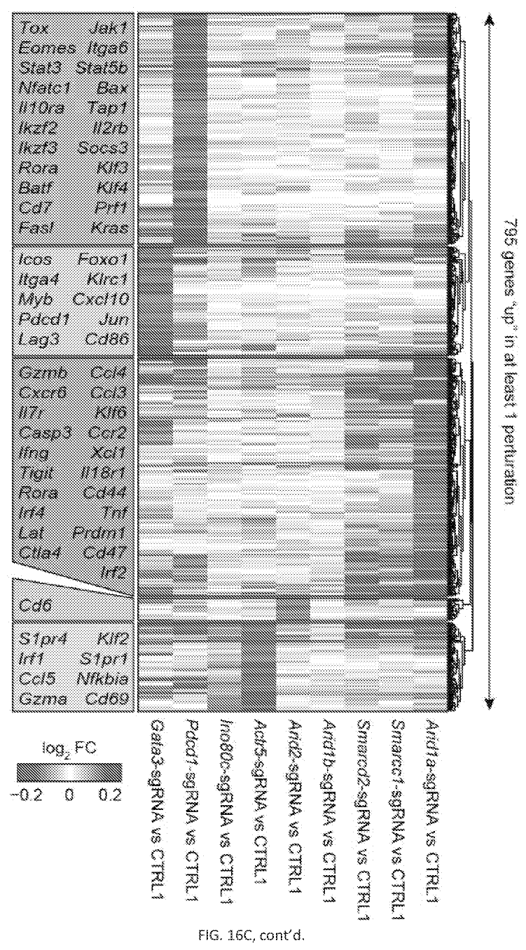

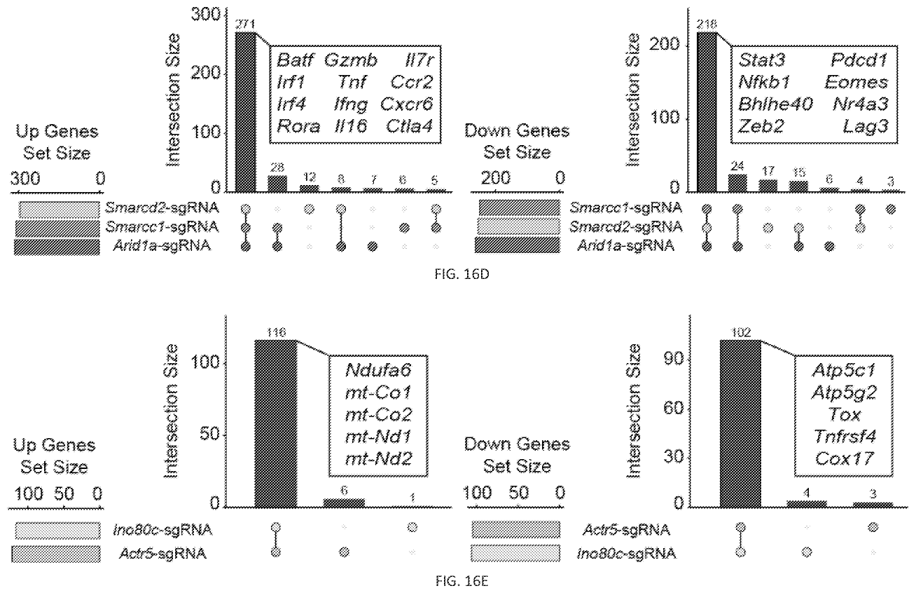

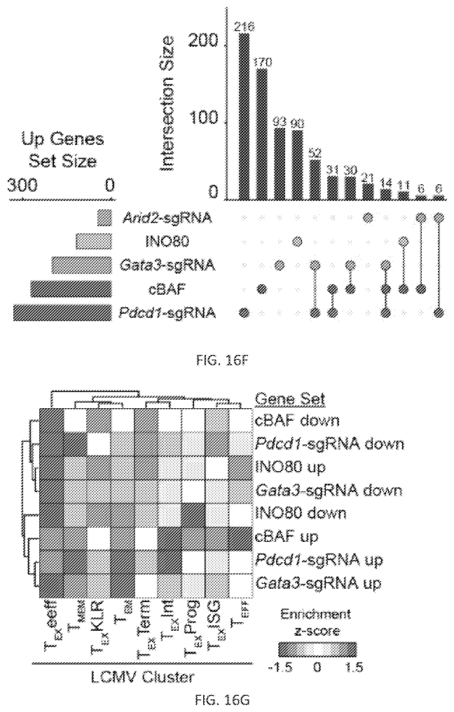

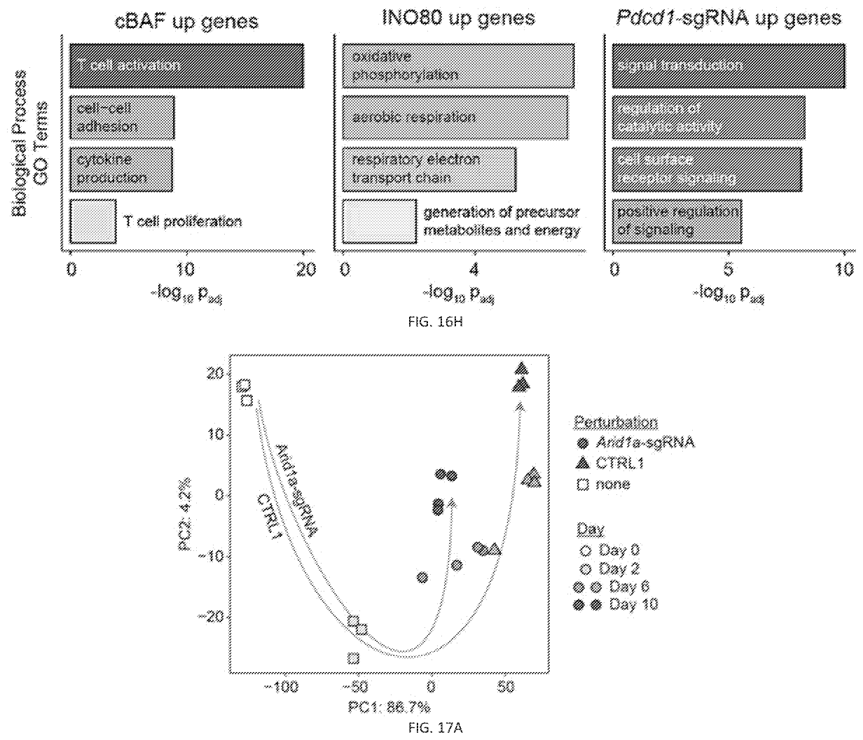

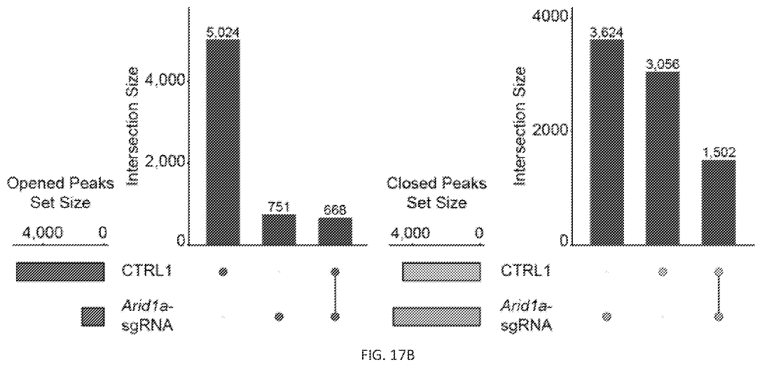

To assign sgRNAs to cells, row z-scores were computed for the ‘CRISPR Guide Capture’ matrix. X-scores were computed for quantifying how enriched each sgRNA was relative to other sgRNAs detected in the same cell. The difference in z-scores was also computed between the most-enriched and second-most enriched sgRNA. Cells which had a maximum sgRNA z-score≥5 and a z-score difference≥2 was determined to contain the guide with maximum z-score, while cells with no sgRNA counts were assigned as “no guide,” and other cells were assigned “multi guide.” The guide assignments were added to the Seurat metadata for downstream processing. Seurat cell cycle scoring was used to predict the cell cycle phase of each single cell. For volcano plot analysis, significantly differential genes were identified as FDR<0.05. For comparisons of different gene sets across perturbations, an addition fold change cutoff was applied of average log₂ FC>0.1 or average log₂ FC<−0.1. For categorization of shared ‘up’ and ‘down’ gene sets within the cBAF and INO80 complexes (analysis shown in FIGS. 16D-16E), the union set of significantly differential genes within each complex was aggregated, and then ‘up’ and ‘down’ genes for each subunit were defined simply as LFC>0 or LFC<0. This strategy was chosen to compare gene sets despite the different amounts of cells collected for each perturbation and resulting difference in statistical power to reach the FDR<0.05 threshold. Seurat gene module scoring was used to convert the LCMV gene sets (consisting of the top 100 marker genes per LCMV cluster) into a gene module score for each cell in the perturb-seq dataset. Gene module scoring was also used to convert the upregulated and downregulated gene sets into module scores for each cell in the expanded LCMV data set, as shown in FIG. 24.

### Example 1

### T Cell Exhaustion In Vitro Model

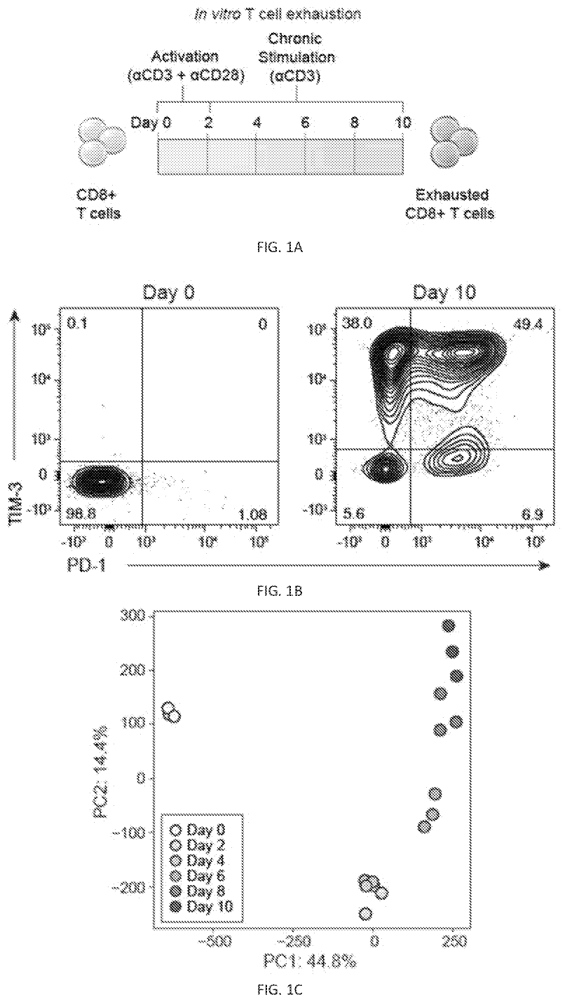

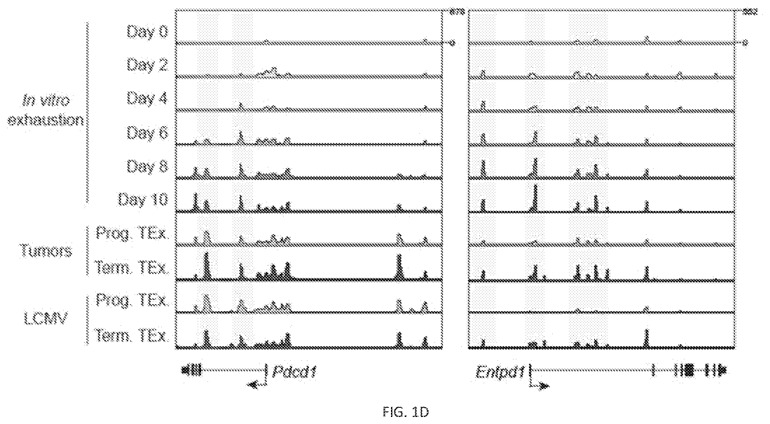

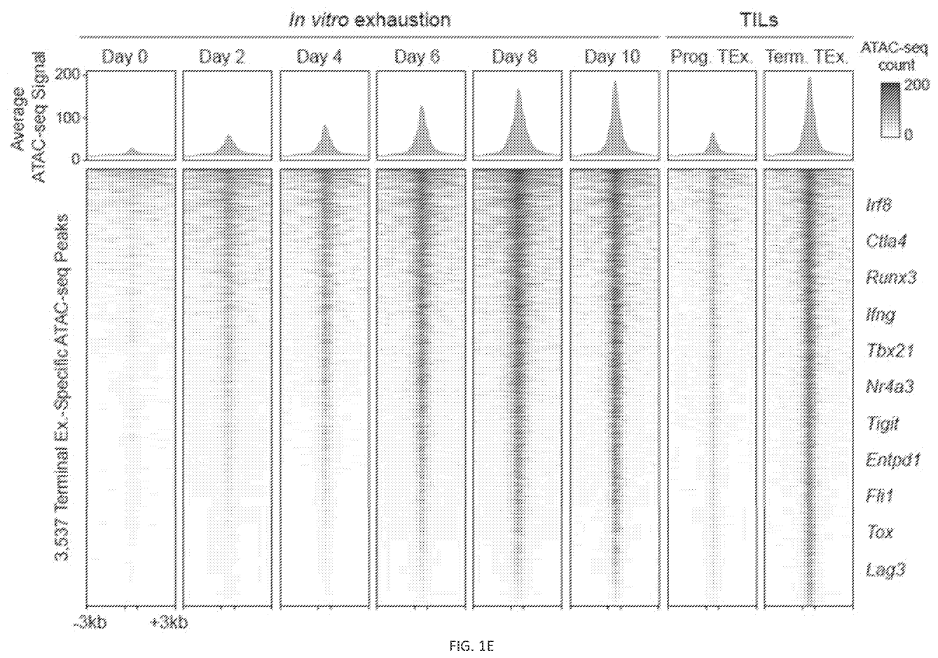

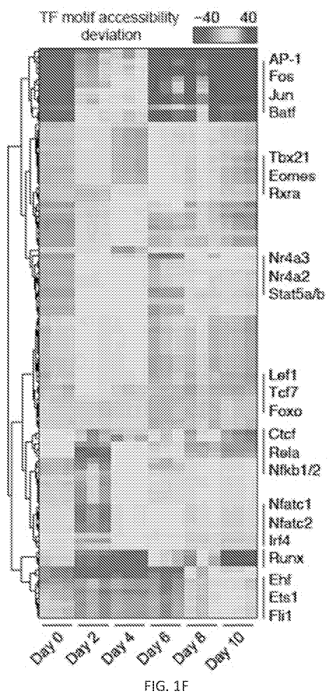

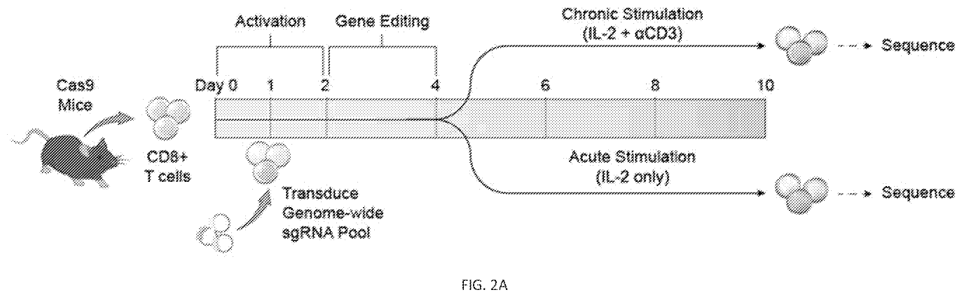

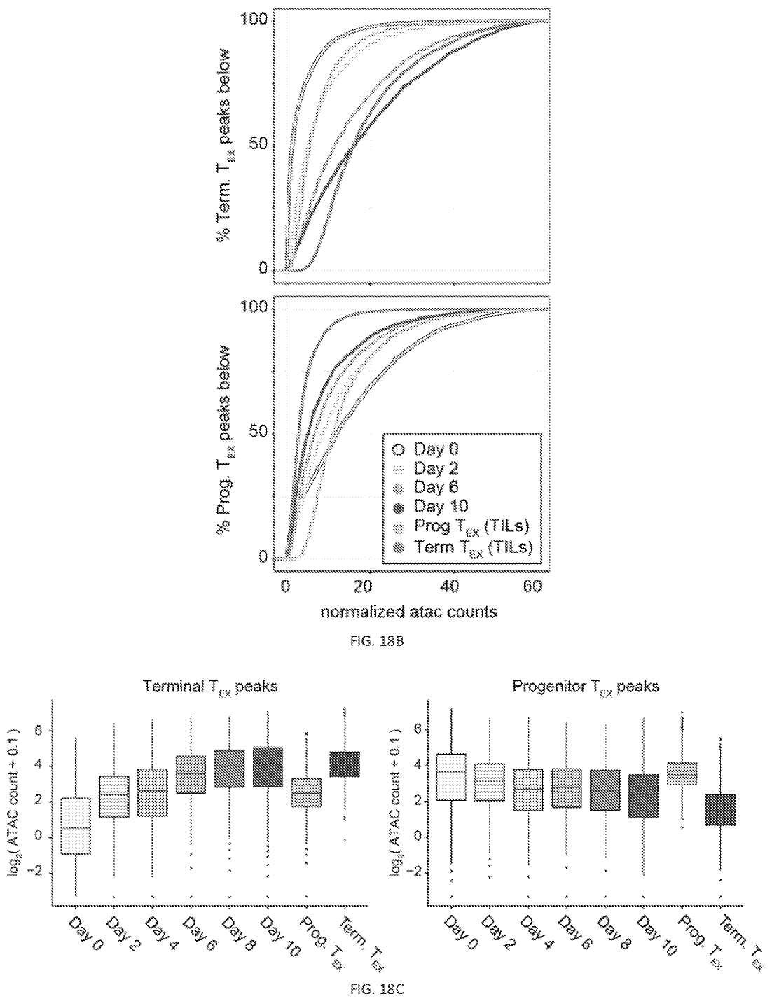

To develop an assay that is amenable to genome-wide CRISPR/Cas9 screening of T cell exhaustion, anti-CD3 antibodies were used to enforce clustering of the T cell co-receptor, CD3, and thereby induce chronic TCR signaling in an antigen-independent manner (FIG. 1A). This model isolates the core determinant of T cell exhaustion-chronic stimulation through the TCR complex—and removes T cell localization and trafficking effects, as well as immunosuppressive factors in the tumor microenvironment, which could be specific to particular in vivo models. Importantly, this assay is scalable; enabling the culture upwards of 108 cells, enabling 1,000× coverage of genome-wide CRISPR sgRNA libraries. Over the course of 8 days of anti-CD3 stimulation (after 2 days of anti-CD3 anti-CD28 activation), a progressive upregulation of the inhibitory receptors, PD-1 and TIM3 was confirmed, and a growth defect in the chronically-stimulated T cells, compared to cells passaged without further stimulation after initial activation (acute stimulation; p<0.0001, unpaired t-test; FIGS. 1B and 6A-6B). Chronically stimulated T cells also exhibited defects in the secretion of IFNγ and TNFα after restimulation with phorbol myristate acetate and ionomycin, compared to acutely stimulated cells (acute: 80% IFNγ+TNFα+, chronic: 1% IFNγ+TNFα+, FIGS. 6C and 18A). Co-culture of OT-1 T cells and B16 tumor cells expressing luciferase and pulsed with the cognate peptide antigen, SIINFEKL (SEQ ID NO: 1), also demonstrated that chronically stimulated cells were impaired in tumor killing in vitro (FIG. 6D). Finally, transplant of chronically stimulated OT-1 T cells into mice bearing B16-OVA tumors demonstrated reduced tumor control in vivo, compared to transplant of acutely stimulated T cells (average tumor size 20 days after transplant: 1,849.6 mm3 (chronic) or 755.0 mm3 (acute); p=0.005, unpaired t-test; FIG. 6E).

An assay for transposase-accessible chromatin with sequencing (ATAC-seq) was performed every two days over the course of chronic stimulation and analyzed global chromatin accessibility profiles. Principal component analysis (PCA) of ATAC-seq profiles showed that PC1 separated naïve cells (Day 0) from all other samples, while PC2 captured a progressive epigenetic polarization of the T cells during chronic stimulation (FIG. 1C). Analysis of individual gene loci, including Pdcd1 and Entpd1, demonstrated an increase in accessibility at known exhaustion-specific regulatory elements (FIG. 1D). The global epigenetic similarity of in vitro stimulated cells to reference T cell exhaustion data from tumors and chronic infection was evaluated. A “terminal exhaustion peak set” was defined as ATAC-seq peaks that are specifically active in terminally exhausted T cells, compared to progenitor exhausted T cells. 3,537 terminal exhaustion ATAC-seq peaks were identified in the B16 melanoma tumor model and 2,346 peaks in the lymphocytic choriomeningitis virus (LCMV) chronic infection model (Log 2 FC≥1; FDR≤0.05; FIGS. 1E and 6F). A comparison of terminal exhaustion peak accessibility in each model with the in vitro exhaustion ATAC-seq data demonstrated that the in vitro assay closely recapitulated global epigenomic changes observed in vivo: 88.6% of ATAC-seq peaks in tumors and 70.1% of ATAC-seq peaks in chronic infection showed a shared increase in accessibility in the in vitro model at Day 10 (FIGS. 1E, 6F and 18B-18C). By contrast, analysis of the 2,926 Progenitor T_{EX} peaks identified in TILs demonstrated that these sites showed decreased accessibility with repeated stimulation (FIGS. 6F, 18B-18C). Chromatin accessibility at transcription factor (TF) binding sites was evaluated using chromVAR, which showed that TF motifs previously associated with terminal exhaustion, including Batf, Fos, Jun, and Nr4a factors were highly accessible in vitro at day 10. Moreover, progressive loss of accessibility at naive and progenitor exhaustion-associated Lef1 and Tcf7 motifs and early dynamic accessibility of NF-κB and Nfat motifs was observed, mirroring the progression of TF activity observed in T cell exhaustion in vivo (FIG. 1F).

### Example 2

### Genome Wide Screening of Genes Implicated in T Cell Exhaustion

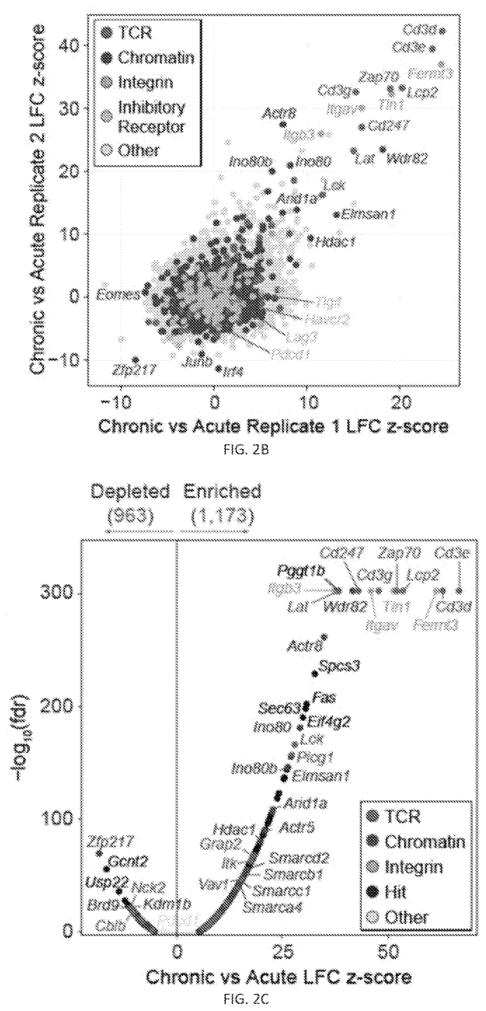

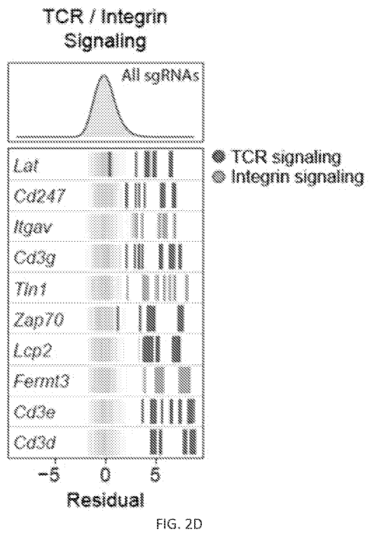

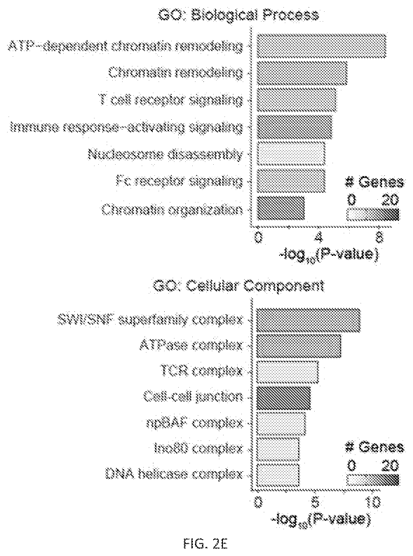

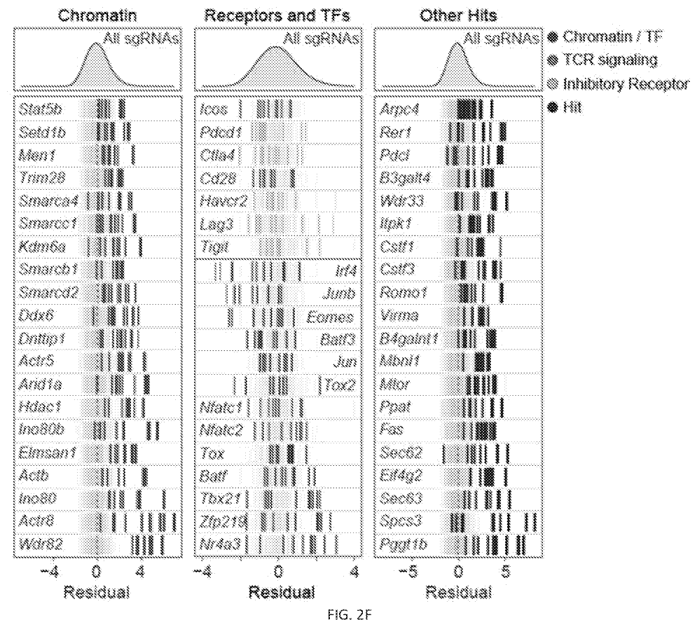

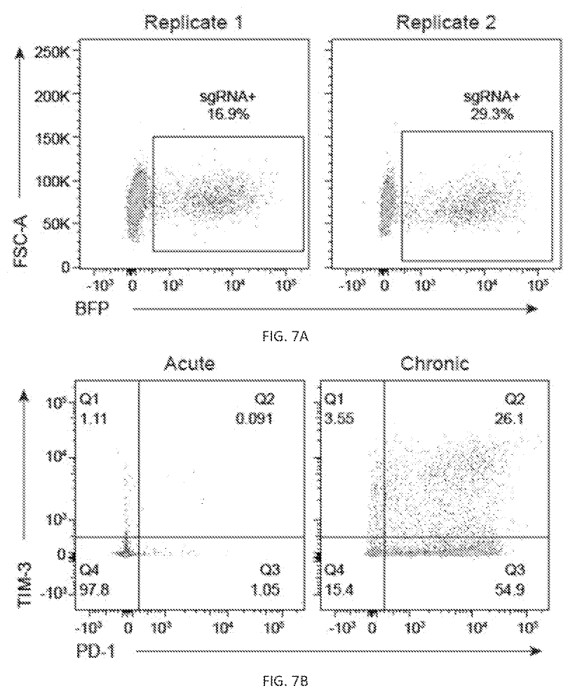

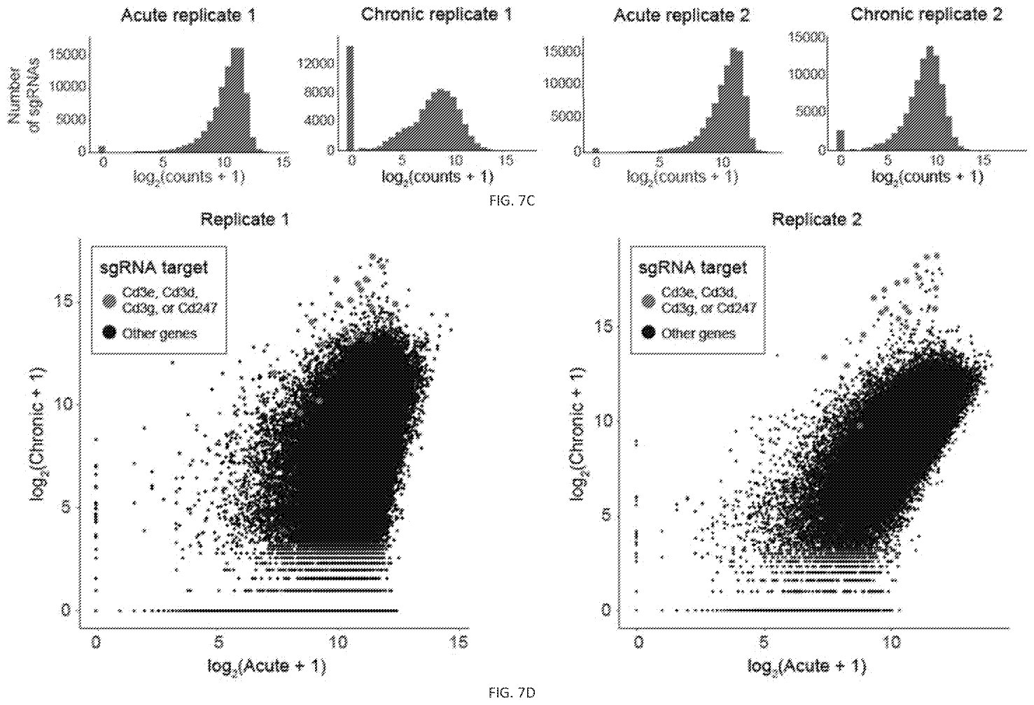

The in vitro exhaustion assay was adapted to be compatible with CRISPR screening by using Rosa26-Cas9 knock-in mice, which constitutively express Cas9-P2A-EGFP (FIG. 2A). Twenty-four hours after T cell isolation, Cas9+CD8+ T cells were transduced with a genome-wide retroviral sgRNA library containing 90.230 sgRNAs. A 48 h delay was introduced between activation and the onset of chronic stimulation to allow time for efficient gene editing and puromycin selection of transduced cells. This modified chronic stimulation protocol caused similar defects in cytokine production after re-stimulation with anti-CD3 or PMA/IO (FIGS. 19A-19B). To identify genes that specifically modulated fitness in the presence of chronic stimulation, the cells were split into acute (IL-2 only) and chronic (anti-CD3 and IL-2) stimulation conditions on Day 4 and both pools were sequenced on Day 10 (FIG. 2A). Replicate screens were prepared and confirmed: (1) transduction of the T cells at low multiplicity of infection (MOI) to optimize single sgRNA targeting of cells (replicate 1: 16.9% sgRNA+ cells, MOI=0.18; replicate 2: 29.3% sgRNA+ cells, MOI=0.35; FIG. 7A), and (2) T cell exhaustion using cell surface phenotype analysis on Day 10 of the chronic culture (FIG. 7B). The guide representation in each condition was analyzed: of the 90,230 sgRNAs present in the plasmid library design, >99% were recovered in each acute sample (acute replicate 1: 89.329 (99.0%); acute replicate 2: 89.625 (99.3%); Gini index mean: 0.39; FIGS. 7C and 19C). The “chronic” samples demonstrated more evidence of selective pressure, with a wider spread in guide counts and a greater number of guides dropping out of the screen (chronic replicate 1: 75,776 sgRNAs detected (83.9%); chronic replicate 2: 87,524 sgRNAs detected (97.0%); Gini index mean: 0.64; FIGS. 7C and 19C). Comparing the counts observed for each sgRNA in the acute and chronic conditions revealed a positive correlation with a small population of sgRNAs dramatically enriched in the chronic condition (FIG. 7D).

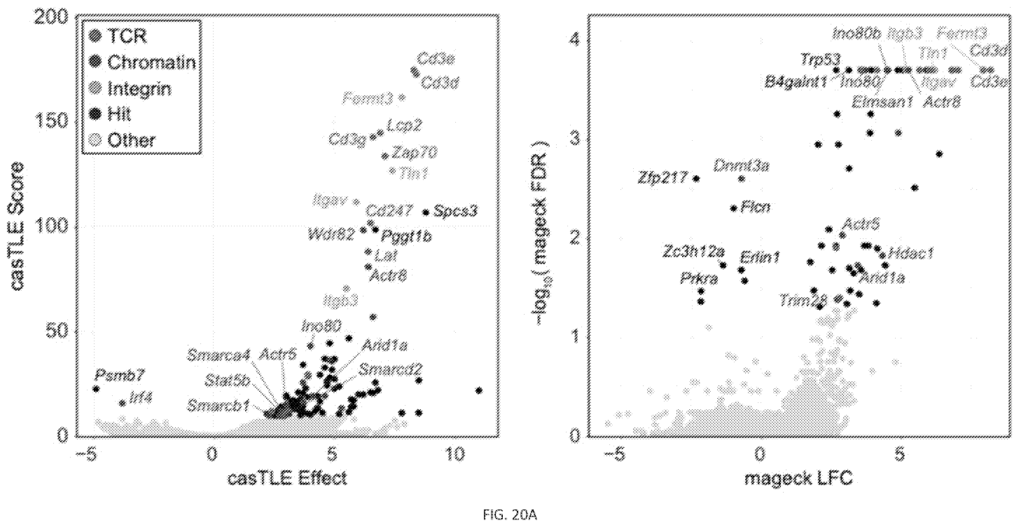

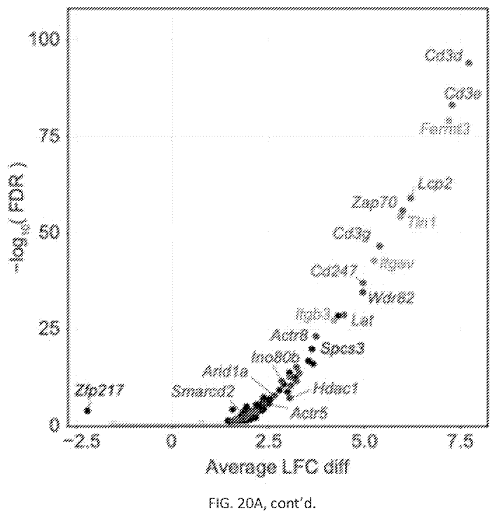

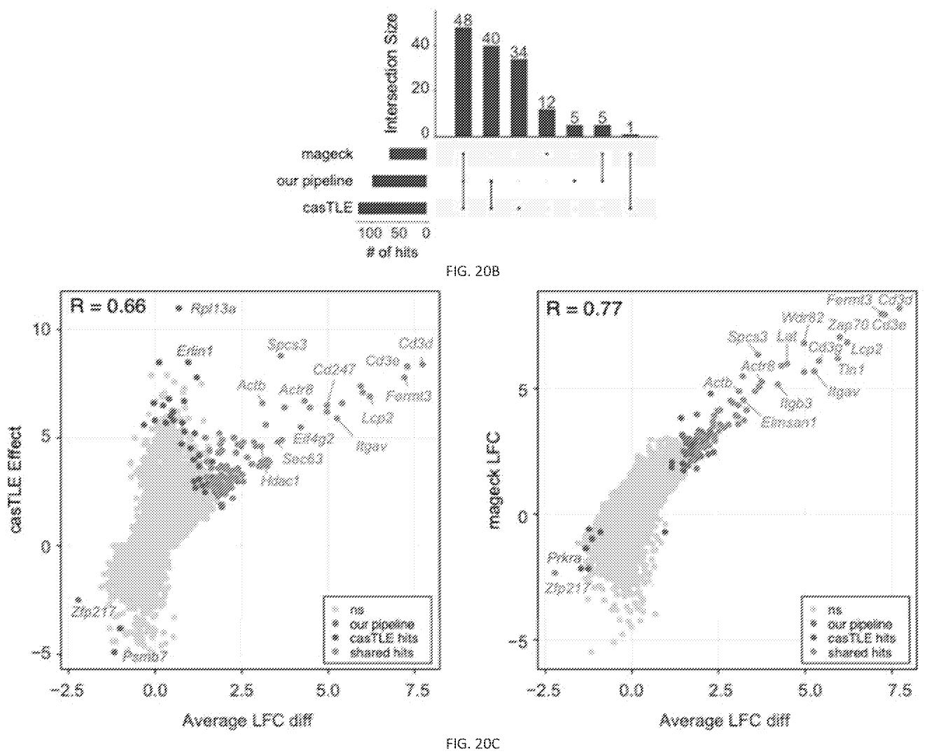

Positive controls for the screen are components of the TCR signaling pathway, since knockout of these factors prevents antigen-driven (or anti-CD3-driven) signaling, and therefore, prevents exhaustion. Accordingly, the enrichments of the CD3 receptor subunits (Cd3e, Cd3d, Cd3g, Cd247, FIG. 7D) were analyzed and a robust enrichment of guides targeting these genes was observed in both replicates. The counts table was normalized and z-scores were computed for each sgRNA, and these sgRNA-level z-scores were merged into a z-score for each gene, as previously described (Flynn et al., (2021) Cell 184, 2394-2411). Merging the replicates yielded an overall z-score and ranking for each gene (“hit” corresponds to FDR<0.001; FIGS. 2B and 2C). This approach was validated by comparing screen hits obtained from two additional widely adopted CRISPR sgRNA enrichment analytical methods, MAGeCK and casTLE, which showed a high correlation between effect size estimates (casTLE effect size correlation: R=0.66; MAGeCK log fold change correlation: R=0.77; FIGS. 20A-20D). A comparison of the genes classified as hits using each method revealed that the largest group of hits were shared by all three methods (“hit” corresponds to FDR<0.05 for the pipeline and MAGeCK or casTLE score>10; FIG. 20B). The retroviral library pool did not contain a control sgRNA set, the normalization strategy (relative to all sgRNAs in the pool) was compared to a strategy that utilizes a set of sgRNAs targeting olfactory receptors that are not expressed or predicted to function in T cells (Gilbert et al., 2014. Cell 159, 647-661). Normalizing sgRNA enrichments to the olfactory receptor sgRNA set modestly boosted the statistical power of the screen results but otherwise had a minimal impact on the results (FIG. 20E).

In addition to Cd3e, Cd3d, and Cd3g, top hits in the screen included other known components of the TCR signaling pathway such as Zap70, Lcp2, Lat, and Lck, as well as cell adhesion and integrin-related genes Fermt3, Tln1, Itgav, and Itgb3 (FIGS. 2B-2D). GO Term analysis of the top 100 positive regulators of exhaustion confirmed that the “T cell receptor signaling pathway” term was highly enriched (padj=7.30×10-6; FIG. 2E). Surprisingly, in addition to TCR-related GO terms, the other top terms were related to epigenetics, including “chromatin remodeling” (padj=1.46×10-6), “chromatin organization” (padj=8.92×10-4), and “nucleosome disassembly” (padj=4.02×10-5; FIG. 2E). Inspection of additional top hits identified a number of chromatin-related factors, including Wdr82, Actr8, Ino80, Actb, Elmsan1, Ino80b, Hdac1, and Arid1a (FIG. 2F, left). The co-stimulatory and inhibitory receptors Icos, Pdcd1, Ctla4, Cd28, Havcr2, Lag3, and Tigit were not significantly enriched by the screen (FIG. 2F, center). The TFs Irf4, Junb, Eomes, and Batf3 were depleted, while Tbx21, and Nr4a3 were modestly enriched, supporting previous demonstrations of their roles in exhaustion (FIG. 2F, center). In contrast, Tox and Tox2 were not significant hits in this screen, demonstrating that deletion of these factors may not improve T cell persistence in vivo, perhaps due to activation-induced cell death (FIG. 2F, center). Similarly, Jun and Barf were not hits, suggesting that while overexpression of these factors improved T cell persistence, deletion did not. Other top hits included genes such as Pggt1b, Spcs3, Sec63, Eif4g2, Sec62, and Fas (FIG. 2F, right). The screen identified negative hits, which represent genes for persistence in the presence of chronic antigen and include Zfp217, Gcnt2, Usp22, Irf4, and Cblb (FIG. 2C).

Cytoscape was used to visualize the protein-protein interaction network of top positive and negative hits (FIG. 3). This analysis confirmed the highly interconnected and enriched network of hits that directly associate with the TCR complex and downstream signaling components, as well as several other protein complexes and functional categories. These included the Ino80 nucleosome remodeling complex (hits included Ino80, Ino80b, Actr5, and Actr8), the Set1C/COMPASS complex that regulates histone methylation (hits included Wdr82, Dpy30, and Setd1b), the SWI/SNF chromatin remodeling complex (hits included Arid1a, Smarcb1, Smarcd2, Smarca4, and Smarcc1), and the mitotic deacetylase (MiDAC) complex comprised of Hdac1, Dnttip1, and Elmsan1. Within the SWI/SNF family members, Smarcc1, Smarcd2, and Smarcb1 are part of the BAF core, which assembles together with Arid1a, Smarca4 (an ATPase), Actb, and other components to form the BAF complex (Mashtalir et al., 2018). Enrichment of the mRNA-processing CSTF complex (Cstf1, Cstf2, and Cstf3), N6-methyladenosine (m6A) RNA modification-related genes, (Zfp217, Rbm15, and Virma), and several hits related to the endoplasmic reticulum and protein secretion (Spcs2, Spcs3, Sec62, Sec63), lipid biosynthesis (Gpi1, Pigv, Dpm3), and the Mitochondrial Complex V (Atp5b, Atp5d, Atp5a1) were also observed.

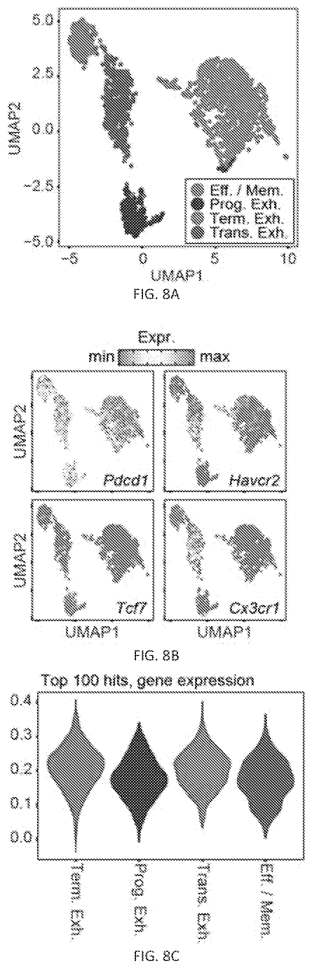

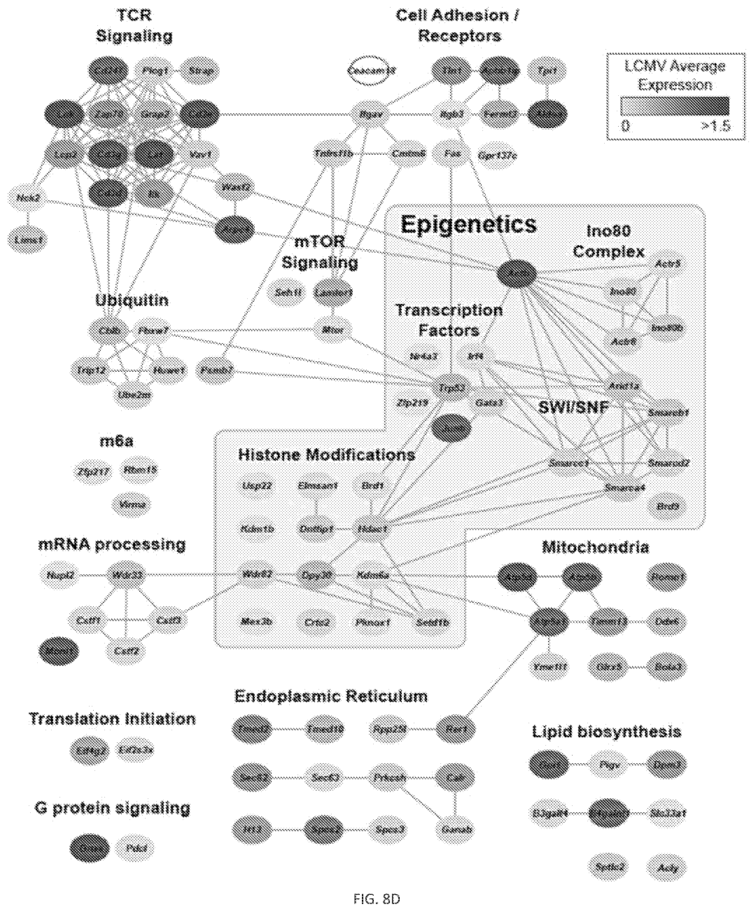

The gene expression patterns in the previously reported single-cell RNA-seq data of exhausted T cells were analyzed in chronic viral infection (Raju et al., (2021) J. Immunol. 206 (12) 2924-2936; FIGS. 8A-8B). This dataset encompasses the key subtypes of progenitor, transitory, and terminally exhausted T cells, and analysis of the top 100 positive hits demonstrated that all factors are detectably expressed in T cells in chronic viral infection (FIGS. 8C-8D). Moreover, all but two of these genes, Tmem253 and Itgb3, were expressed early during exhaustion (98 of the top 100 hits detectably expressed in progenitor exhausted T cells) and remained relatively stable across exhausted subtypes, suggesting that disruption of epigenetic factors and other hits altered the molecular course of T cell exhaustion, rather than reversing exhaustion only after terminal differentiation (FIG. 8C).

### Example 3

In Vivo CRISPR Screens Identify Epigenetic Factors that Limit T Cell Persistence in Tumors

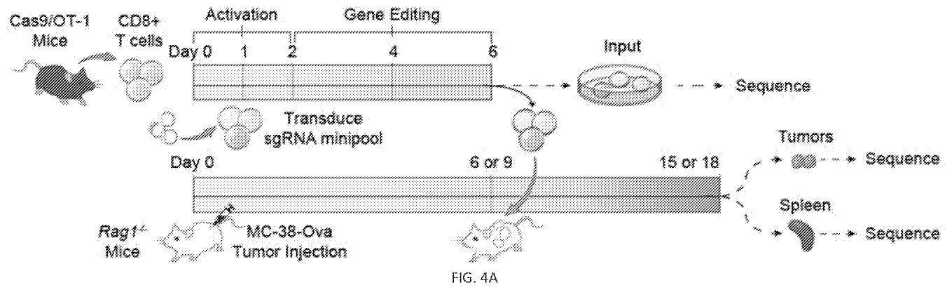

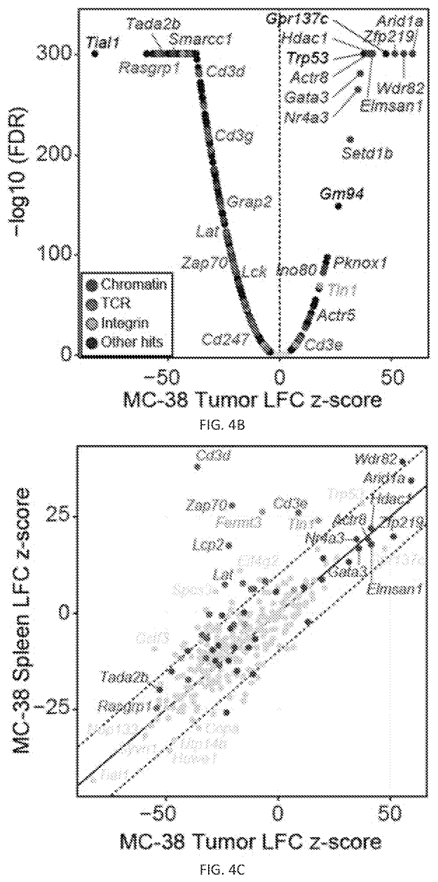

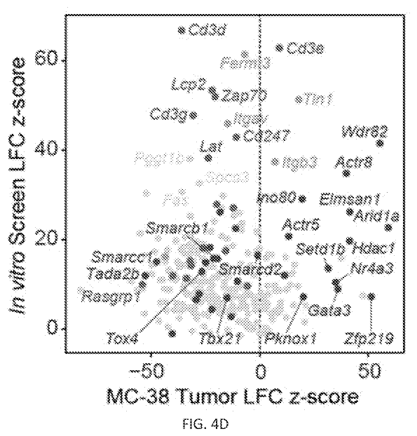

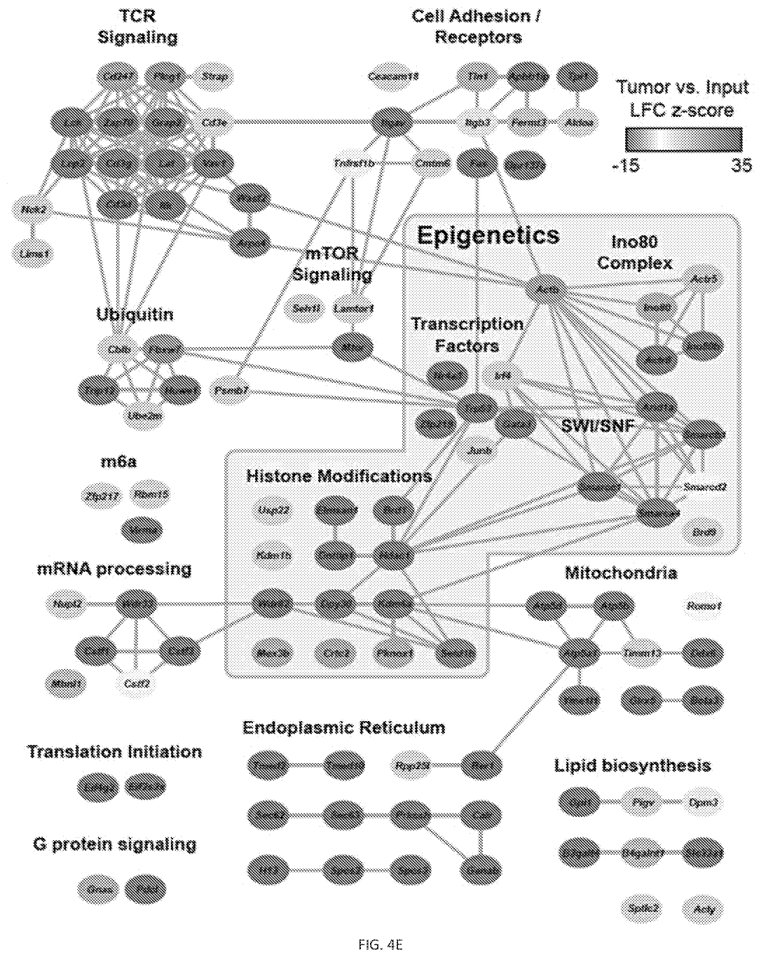

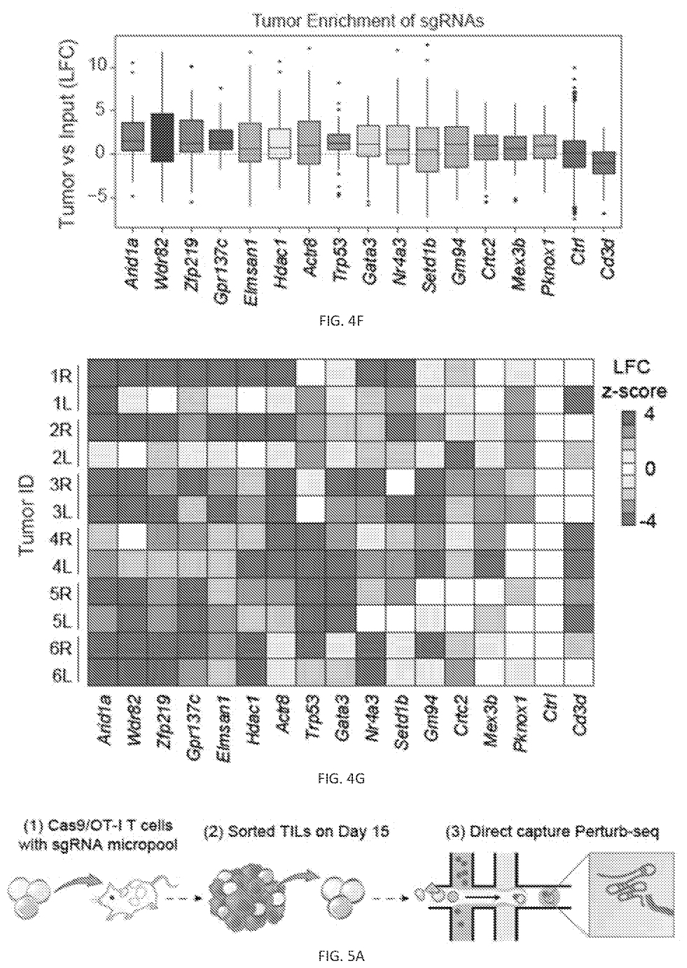

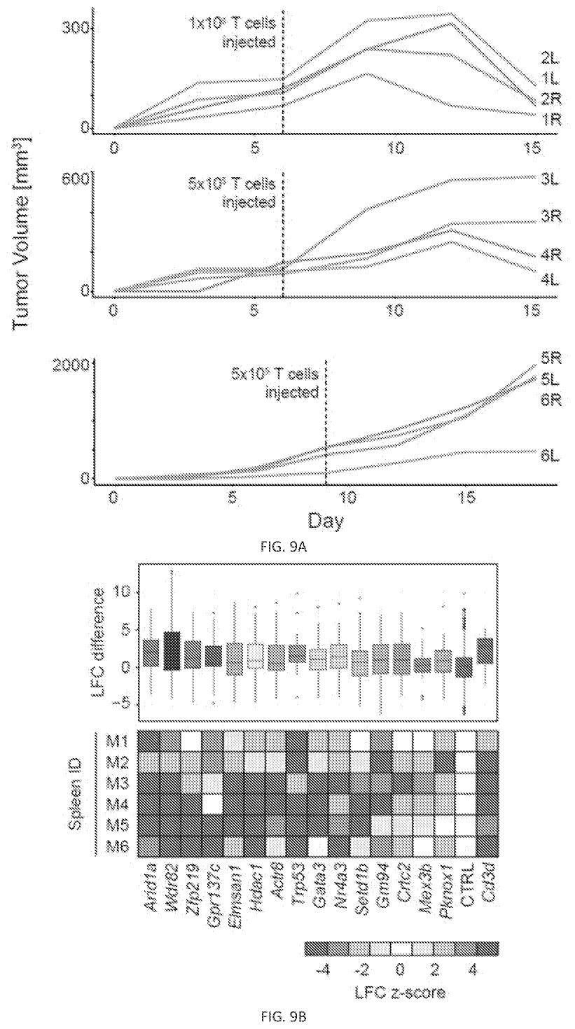

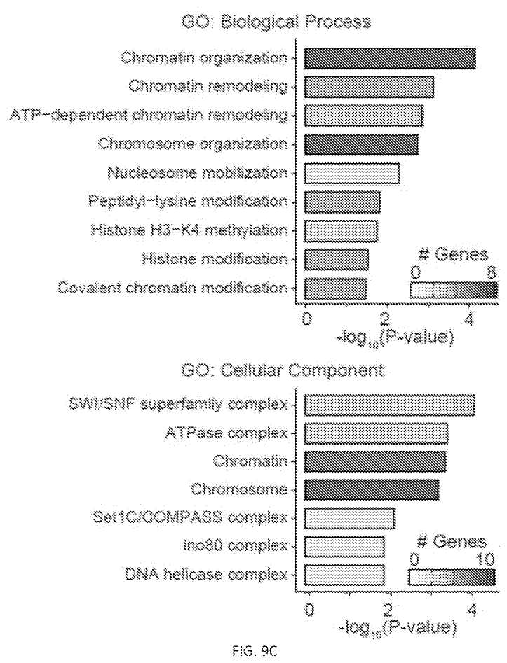

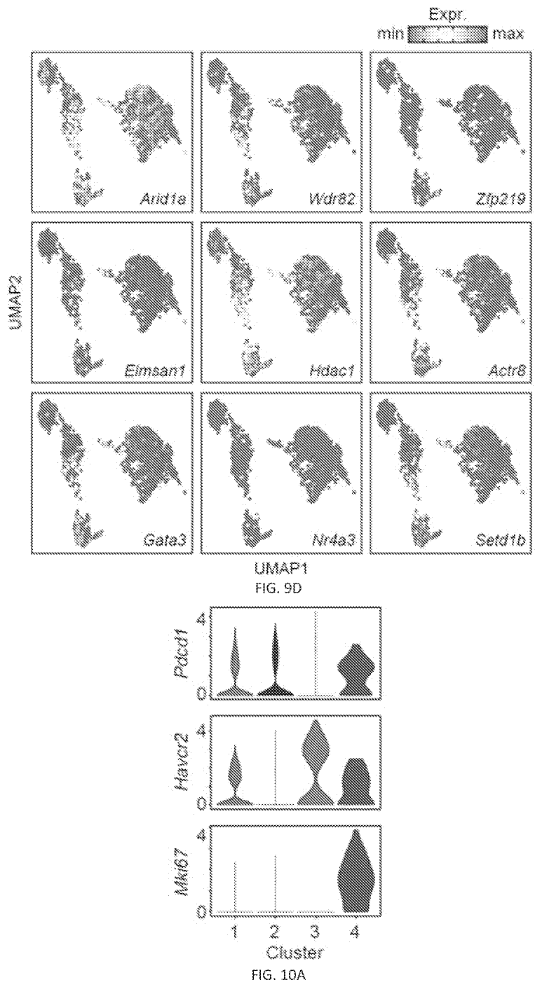

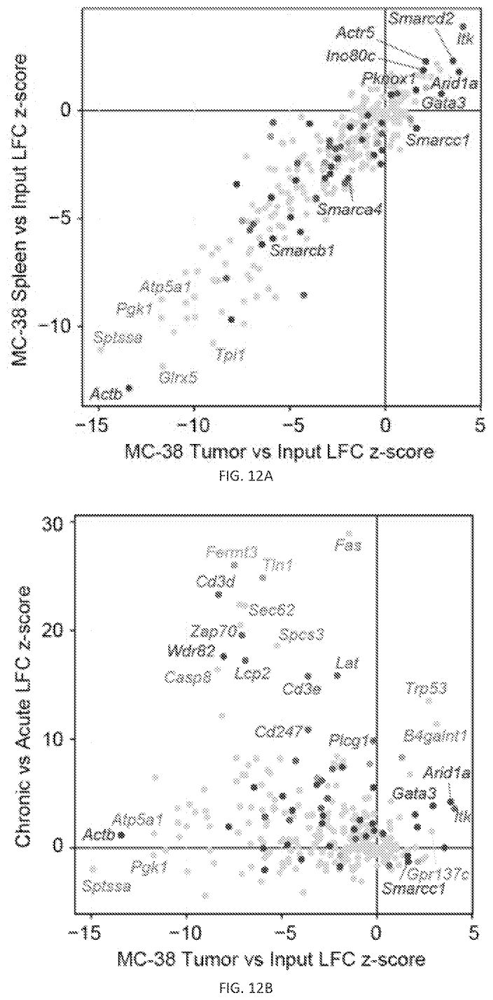

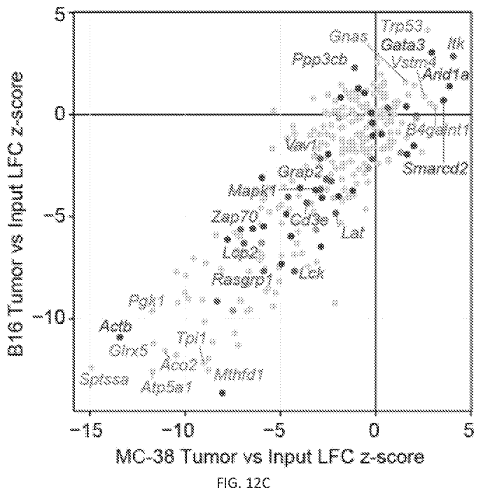

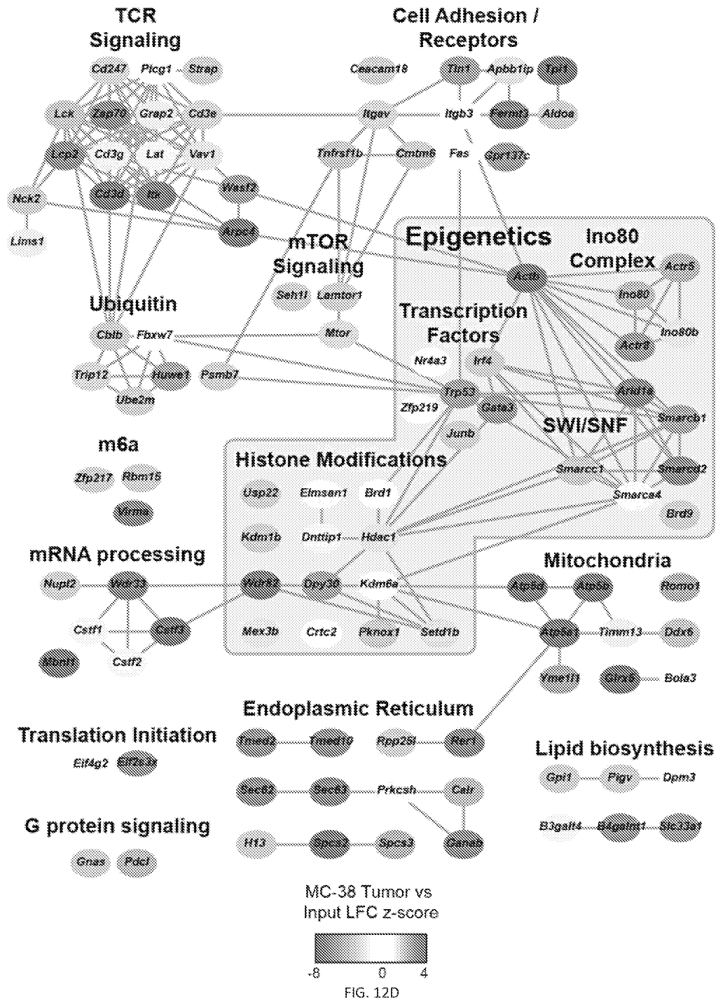

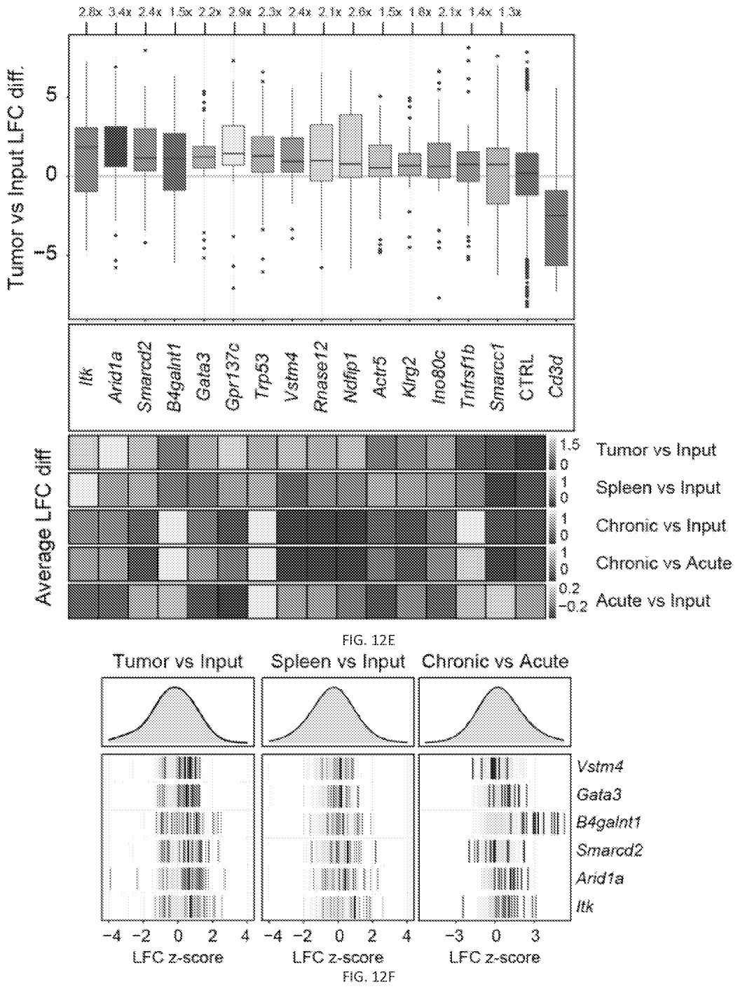

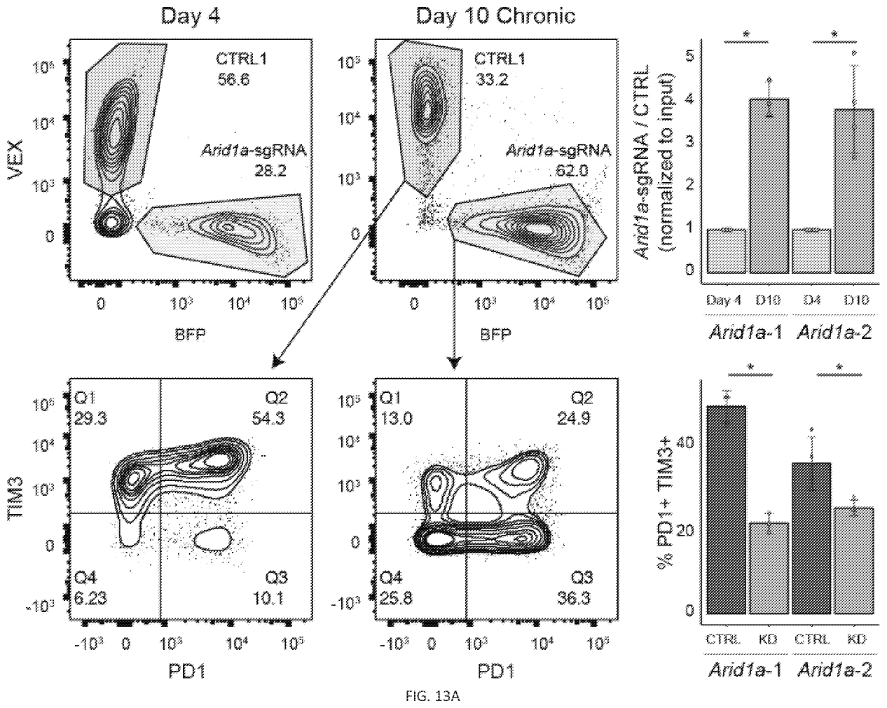

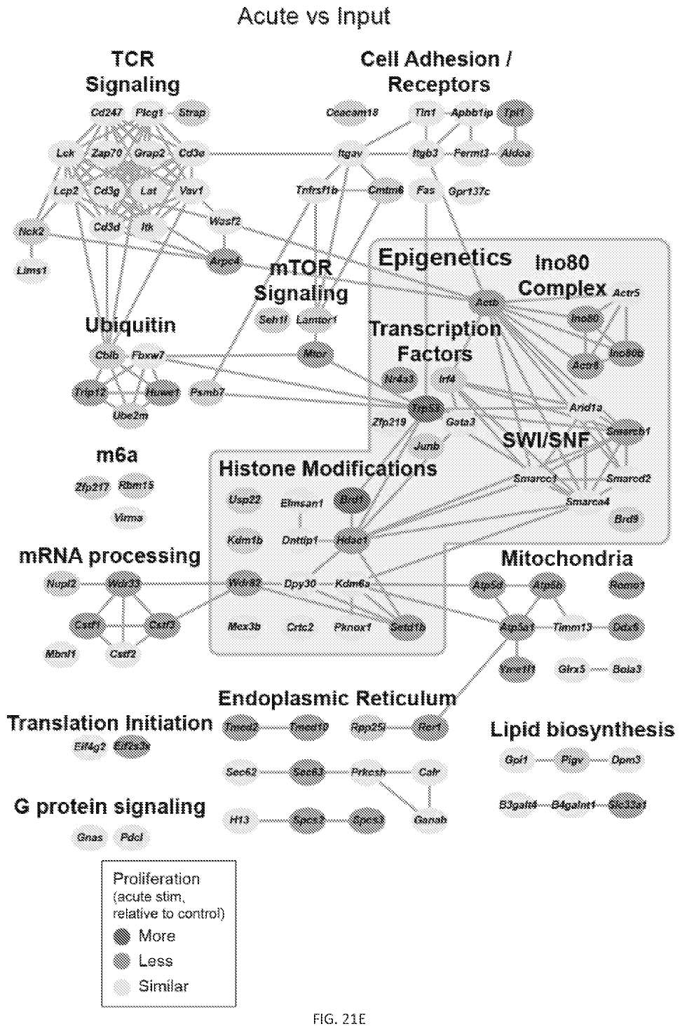

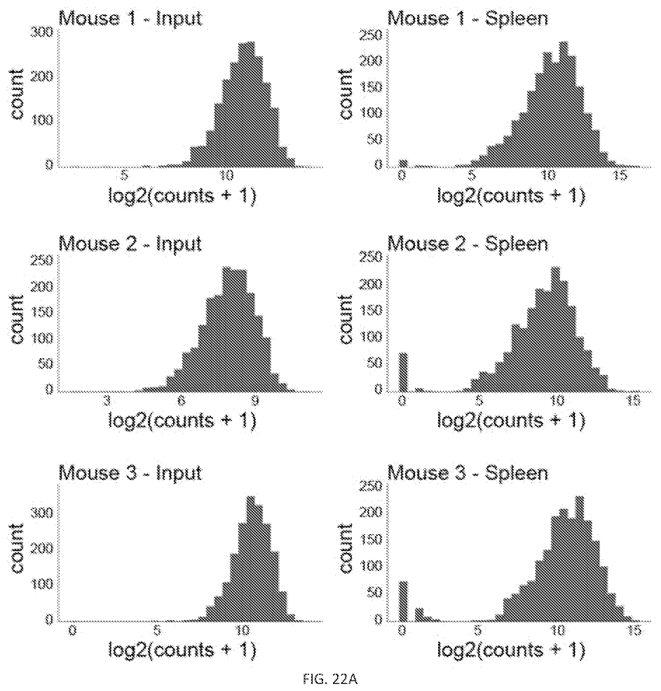

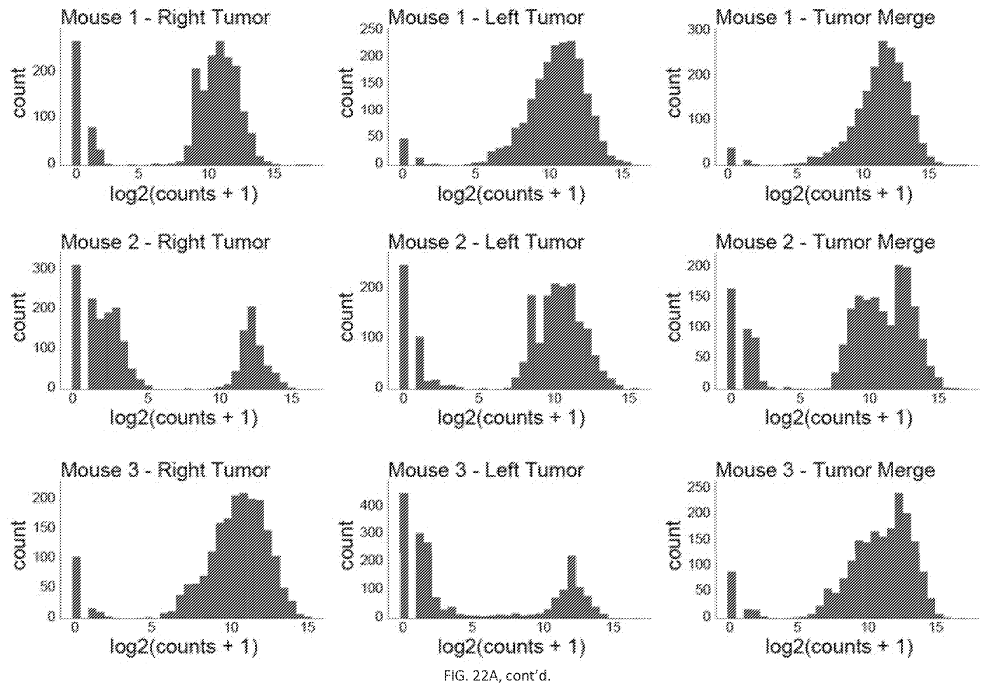

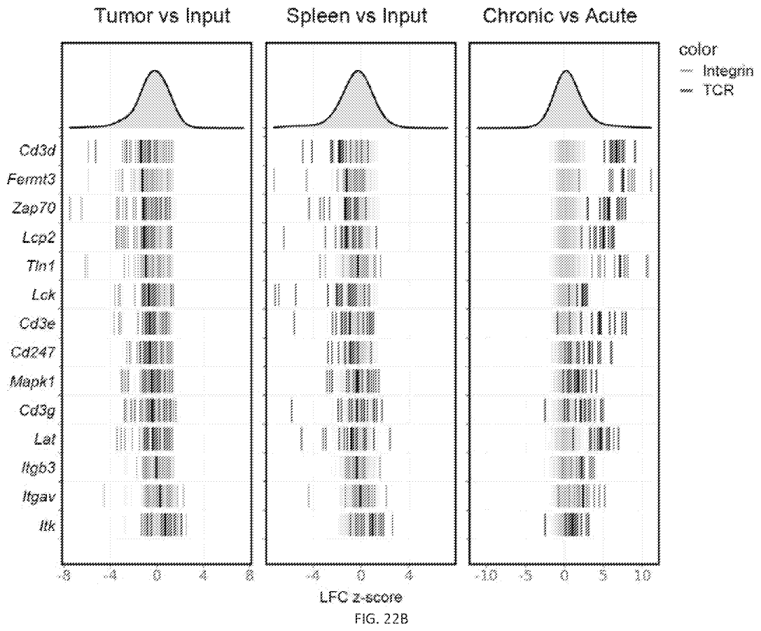

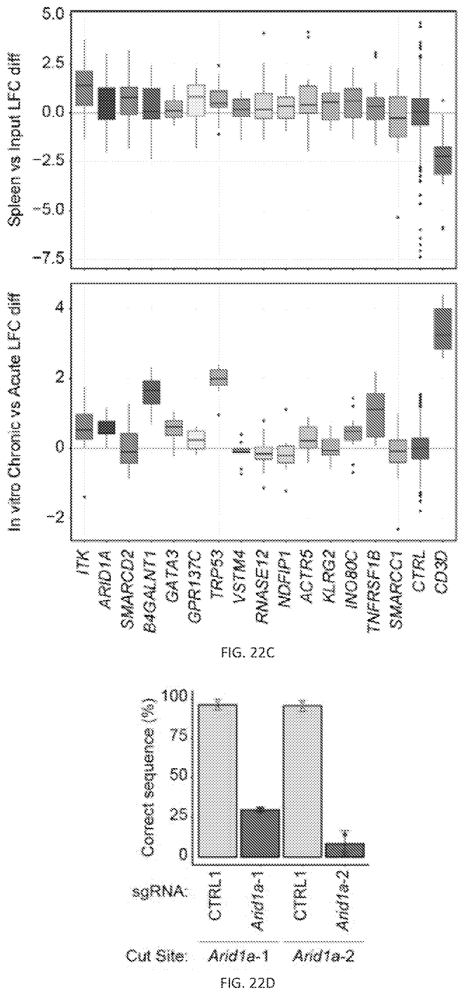

A custom pool of 2,000 sgRNAs was created, which included sgRNAs that targeted the top 300 hits (6 sgRNAs per gene), as well as 100 non-targeting and 100 single-targeting controls. The sgRNA pool was introduced into Cas9/OT-1 T cells, to remove functional variability due to differing TCR sequences. On day 0, bilateral MC-38 colon adenocarcinoma tumors that ectopically expressed ovalbumin were injected into Rag1−/− mice and CD8+ T cells were isolated from Cas9/OT-1 mice. On day 1 the T cells were transduced with the custom minipool (FIG. 4A). Three different T cell dosing protocols were used to monitor protocol-specific knockout effects: group 1 received 1×10⁶ T cells per mouse 6 days after tumor inoculation, group 2 received 5×10⁵ T cells per mouse 6 days after tumor inoculation, and group 3 received 5×10⁵ T cells per mouse 9 days after tumor inoculation (FIG. 9A). A T cell dose-dependent reduction in tumor size was observed. The tumors and spleens of mice were harvested on day 15 (groups 1 and 2) or day 18 (group 3) and the sgRNAs present in each tissue were sequenced (FIGS. 9A and 22A). Finally, sgRNA enrichments were computed and the results were merged from all mice to create an aggregate tumor LFC z-score and spleen LFC z-score for each gene, relative to the control distribution (FIGS. 4B and 12A-12C).

sgRNAs targeting the TCR complex and signaling genes were analyzed, since cells containing these guides should have an impaired ability to recognize antigen and thus be depleted in tumors. Indeed, sgRNAs targeting nearly all of the previously identified TCR and integrin signaling-related hits were depleted in tumors relative to the spleen (FIG. 4C). Similarly, genes in a number of other functional categories were also depleted in both tumors and spleens, likely indicating a general proliferation impairment of these knockouts in vivo (FIGS. 12A and 12D).

However, in contrast, a select group of in vitro hits were strongly enriched in both the tumor and spleen and were largely composed of chromatin-related factors (FIGS. 4B-4C and 12A-12C). Among the top hits enriched in tumors were the TFs, Nr4a3 and Gata3. The functional role of 8 epigenetic regulators, including the Ino80 complex factors, Ino80, Actr5, and Actr8, the Set1C/COMPASS complex members, Wdr82 and Setd1b, the BAF complex member, Arid1a, and the MiDAC complex members, Hdac1 and Elmsan1 were identified (FIG. 4B). Gata3 is a transcription factor which was previously demonstrated to regulate the development of T cell exhaustion, and importantly, deletion of this factor improves T cell function, persistence, and tumor control in vivo. The remaining hits have not been studied in T cell exhaustion or immunotherapy contexts. Visualizing the tumor enrichments of each gene in the context of the Cytoscape network revealed that many of the positive hits in vivo were epigenetic factors, including subunits of two chromatin remodeling complexes, the INO80 complex, (subunits Ino80c and Actr5), and the BAF complex (subunits Arid1a, Smarcd2, and Smarcc1; FIG. 12D). Other categories were also represented, such as the G-protein coupled receptor, Gpr/37c, an enzyme involved in ganglioside biosynthesis, B4galnt1, and the IL-2 inducible T cell kinase, hk. The sgRNA enrichments of the top positive hits were calculated and compared to input controls. Each gene knockdown improved T cell accumulation in tumors by up to 3.4-fold. For comparison, T cells lacking Cd3d were depleted 6.7-fold and T cells lacking Cd3e were depleted 3.3-fold, demonstrating that targeting the top hits substantially improved T cell persistence in tumors (FIGS. 12E and 22C). Furthermore, the persistence advantage of each knockout was similar in the tumor and the spleen, and no perturbation except Trp53 displayed substantially improved fitness in the absence of chronic TCR stimulation (during acute stimulation in vitro), again demonstrating the specificity of the CRISPR screen strategy to identify perturbations that improve T cell persistence only in the setting of chronic antigen stimulation, rather than improving general T cell fitness (FIGS. 12E-12F and 22C).

A role for a previously uncharacterized TF. Zfp219, in T cell function in vivo was also identified. sgRNAs targeting these epigenetic factors were more highly enriched in tumors than the sgRNAs targeting Nr4a3 and Gata3. GO term analysis and visualization of in vivo sgRNA z-scores in the context of the cytoscape network confirmed that functional categories related to chromatin and nucleosome remodeling and organization, and histone modifications were the major enriched group of genes (FIGS. 4E and 9C-9D). sgRNA enrichments for the top 15 in vivo hits in tumor and spleen samples were analyzed separately, and across T cell dosing protocols. sgRNAs targeting each gene were reproducibly enriched across individual mice, organs, and tumor sizes (FIGS. 4F, 4G, 9B). Of note, deletion of each epigenetic factor improved T cell accumulation in tumors by approximately ˜3-5 fold, compared to control sgRNAs, which is comparable to the scale of T cell depletion that was observed for T cells lacking a component of the CD3 co-receptor, Cd3d, demonstrating the significant improvement in T cell function that is mediated by deletion of these genes (FIG. 4F).

### Example 4

### In Vivo Perturb-Seq of T Cell Exhaustion Factors in TILs

Perturb-seq, which captures CRISPR perturbation and transcriptome in single cells was used to understand the molecular mechanisms driving improved T cell function in each knockout identified by the in vivo CRISPR screens. Specifically, direct-capture Perturb-seq was used because it does not require a vector with a barcode sequence separate from the sgRNA, or other modifications to standard sgRNA vectors, and thus was immediately compatible with the retroviral reagents. A third custom sgRNA pool (micropool) of sgRNAs was designed by prioritizing genes that: (1) preferentially persisted in the in vitro assay, (2) preferentially proliferated and infiltrated tumors in vivo, and (3) were chromatin-related proteins or TFs. Based on these criteria, nine genes were selected for Perturb-seq analysis: Wdr82, Setd1b, Arid1a. Actr8, Ino80, Hdac1, Elmsan1, Nr4a3, and Zfp219. The sgRNA pool contained two guides per gene, as well as two non-targeting and two single-targeting control guides, for a total of 22 sgRNAs. To ensure similar representation of all guides, pairs of primers containing the 20 bp variable sgRNA sequences were individually annealed, which were then pooled and cloned together into retroviral vector pMSCV.

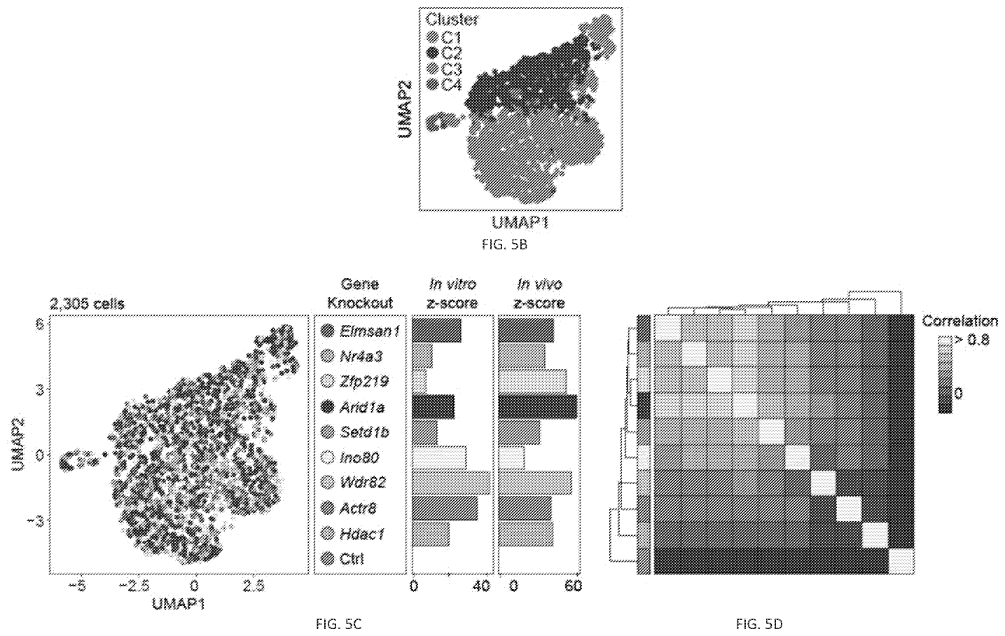

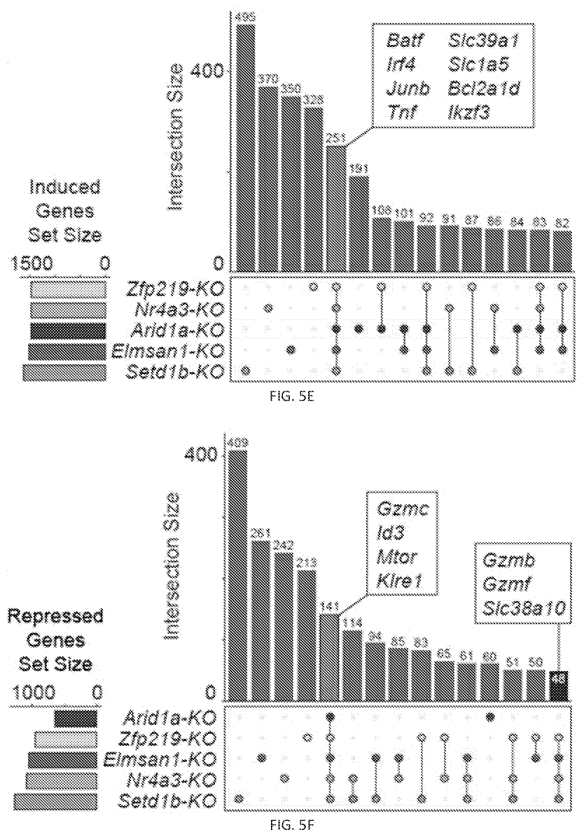

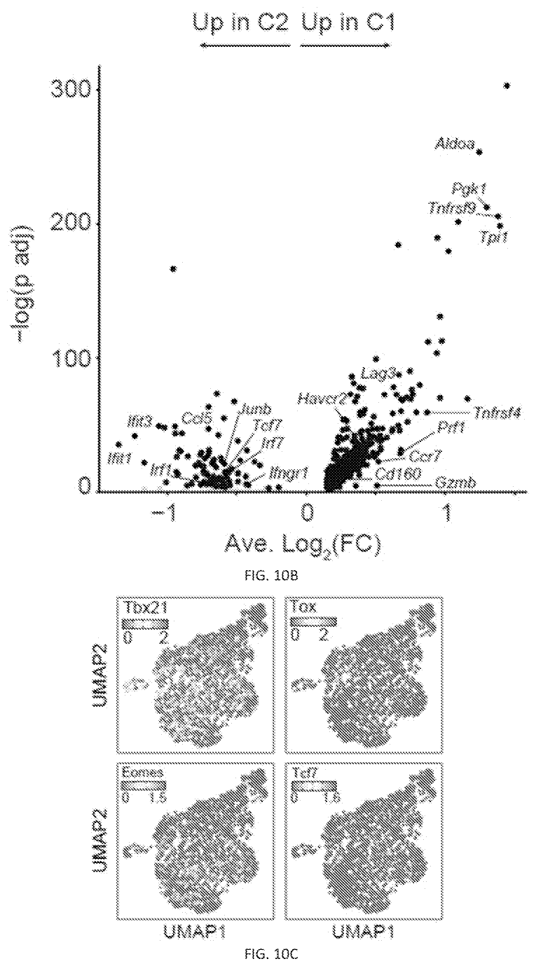

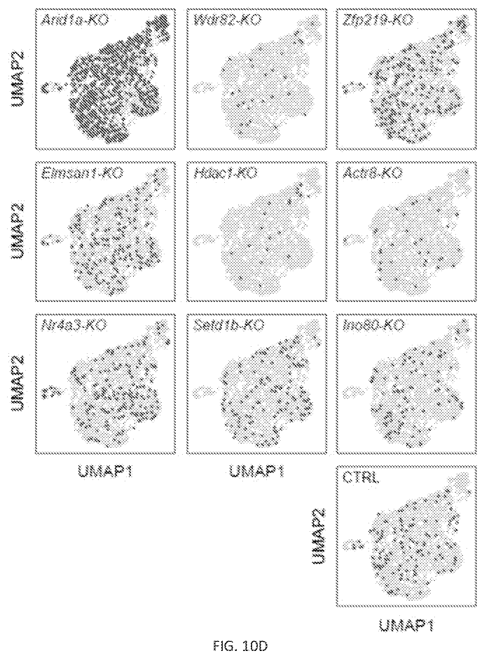

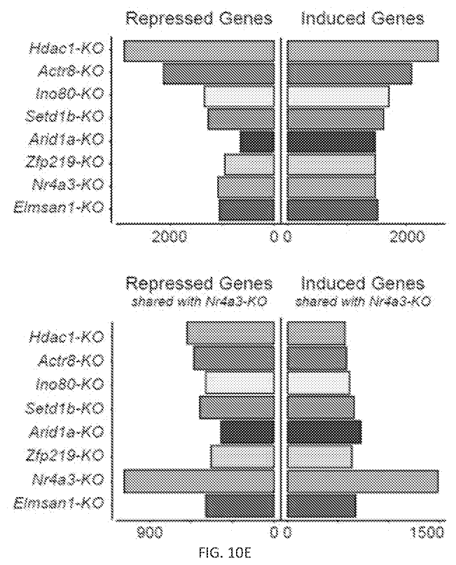

A similar in vivo T cell protocol was performed as previously described for the larger CRISPR screen: CD8+ T cells were isolated from Cas9/OT-1 mice, transduced with the sgRNA micropool, and then transplanted into Rag1−/− mice bearing MC-38 ovalbumin tumors. Nine days later, tumors were harvested, TILs were isolated, and direct-capture Perturb-seq was used to read out sgRNA identity and gene expression profiles simultaneously using the 10× Genomics 5′ gene expression platform (FIG. 5A). After quality control filtering, high-quality scRNA-seq profiles were obtained from 2,305 cells, and scRNA-seq clustering and dimensionality reduction identified 4 clusters (FIG. 5B). All cell clusters contained cells expressing moderate levels of Pdcd1 and Havcr2, indicating that they represented exhausted T cells in the tumor microenvironment (FIG. 10A). Comparing marker genes between Cluster 1 and Cluster 2 revealed that Cluster 1 had higher expression of costimulatory molecules, including Tnfrsf9 (encoding 4-1BB) and Tnfrsf4 (encoding OX40), the cytotoxic molecules, Gzmb and Prf1, and inhibitory receptors, including Lag3, Havcr2, and Cd160 (FIG. 10B). In contrast, Cluster 2 had higher expression of the progenitor exhaustion genes, Tcf7, Ifngr1, and Ccl5, as well as several interferon response genes, including Ifit1, Ifit3, Irf1, and Irf7 (FIGS. 10B-10C). Cluster 1 and Cluster 2 contained the vast majority of the cells, while Cluster 3 contained a small population of cells with a higher percentage of mitochondrial reads, and Cluster 4 contained a small number of proliferating cells, marked by Mki67 expression (FIG. 10A).

A high-confidence sgRNA identity was determined for each cell by considering the sgRNA by cell counts matrix and computing row (cell) z-scores (FIGS. 5C and 10D). Any cell with a maximum sgRNA z-score>3 was determined to contain the guide with maximum z-score, while cells with no sgRNA counts were assigned as “no guide,” and cells with a lower maximum z-score were assigned “multi guide”. The average gene expression profile for cells with a given perturbation was computed, the gene expression profile of control cells was subtracted, and differential gene expression profiles were correlated across the different perturbations (FIG. 5D). Overall, cells with each gene KO exhibited large-scale changes in their transcriptional profile (1,474 to 2,533 induced genes LFC>0.1; 643 to 2,900 repressed genes LFC<−0.1; FIG. 10E). Cells depleted of Elmsan1, Nr4a3, Zfp219, Arid1a, or Setd1b highly-correlated changes in gene expression (R>0.5 for all pairs of perturbations), indicating a convergent phenotype induced by knockout of these factors (FIG. 5D). A set of “induced” or “repressed” genes for each perturbation, relative to control cells were defined, and all perturbations shared approximately half of the genes induced or repressed by Nr4a3 (FIG. 10E). Overlap of gene sets were visualized for five perturbations—Zfp219-KO, Nr4a3-KO, Arid1a-KO, Elmsan1-KO, and Setd1b-KO—and found that the largest group of overlapping genes was shared across all perturbations (FIGS. 5E-5F). These genes included: (1) cytotoxic molecules, cytokines, and cytokine receptors, including upregulation of Tnf, Ifng, and Il7r, and downregulation of Gzmb, Gzmc, and Gzmf, (2) exhaustion-relevant TFs, including upregulation of Batf, Irf4, and Klf2, and downregulation of Tbx21, (3) and amino acid transporters and other metabolic genes, including upregulation of Sic1a5 (ASCT2, imports glutamine), Slc38al (SNAT1, neutral amino acids), and Slc38a2 (SNAT2, neutral amino acids). Although isolated changes in inhibitory receptor expression were observed, consistent changes were not observed across perturbations (FIG. 5G).

### Example 5

### TILs Mini-Pool CRISPR Screens Validate Genetic Regulators of T Cell Exhaustion In Vitro

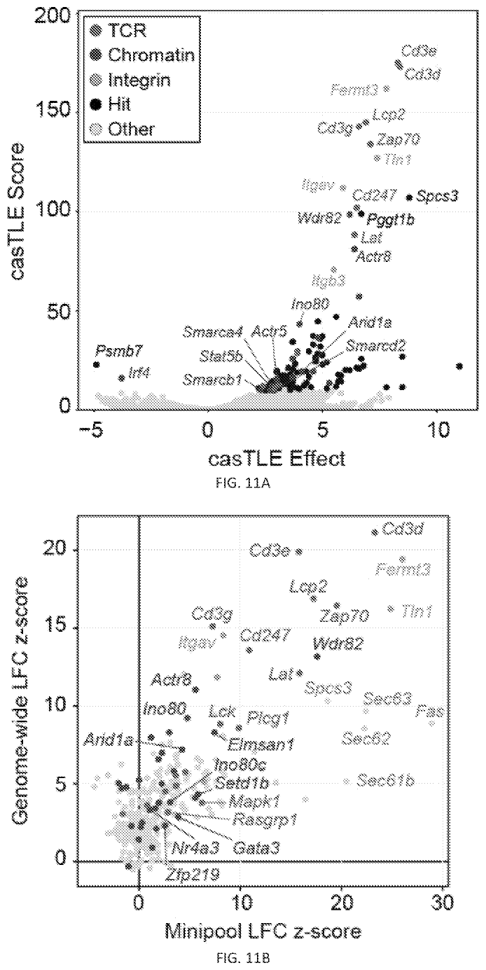

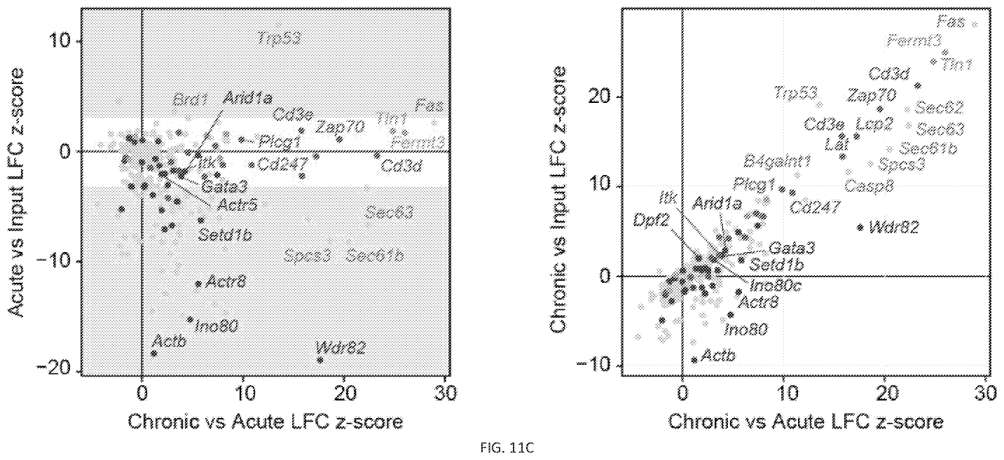

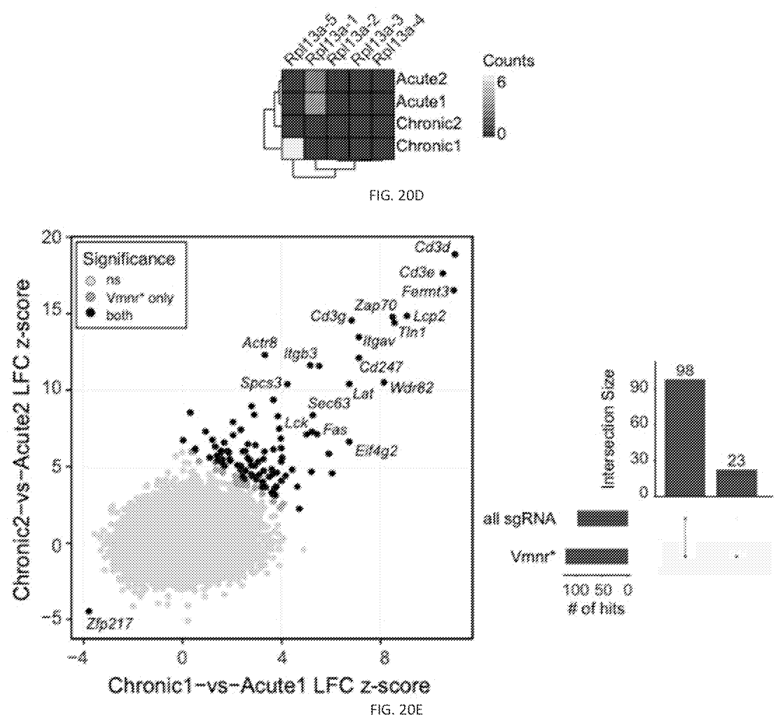

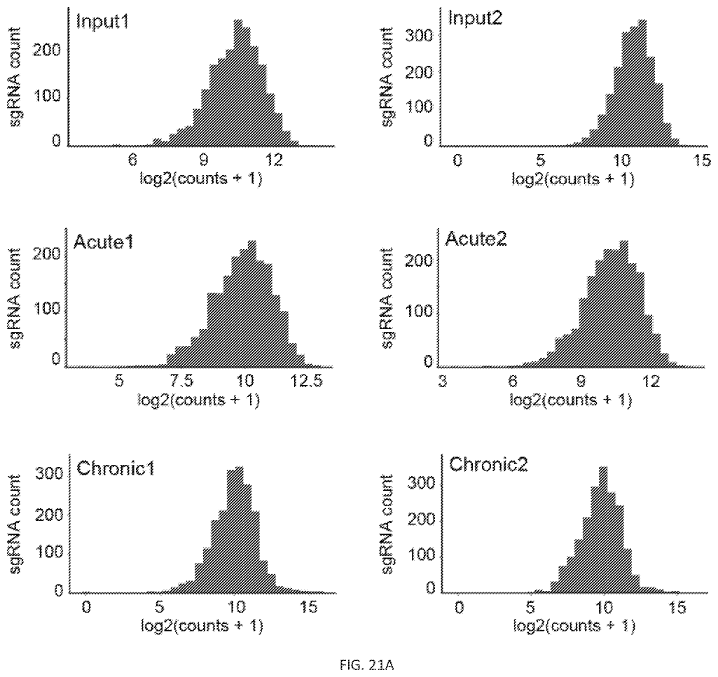

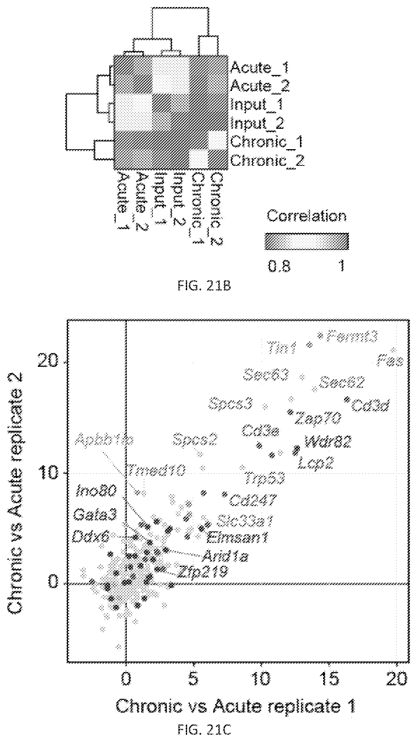

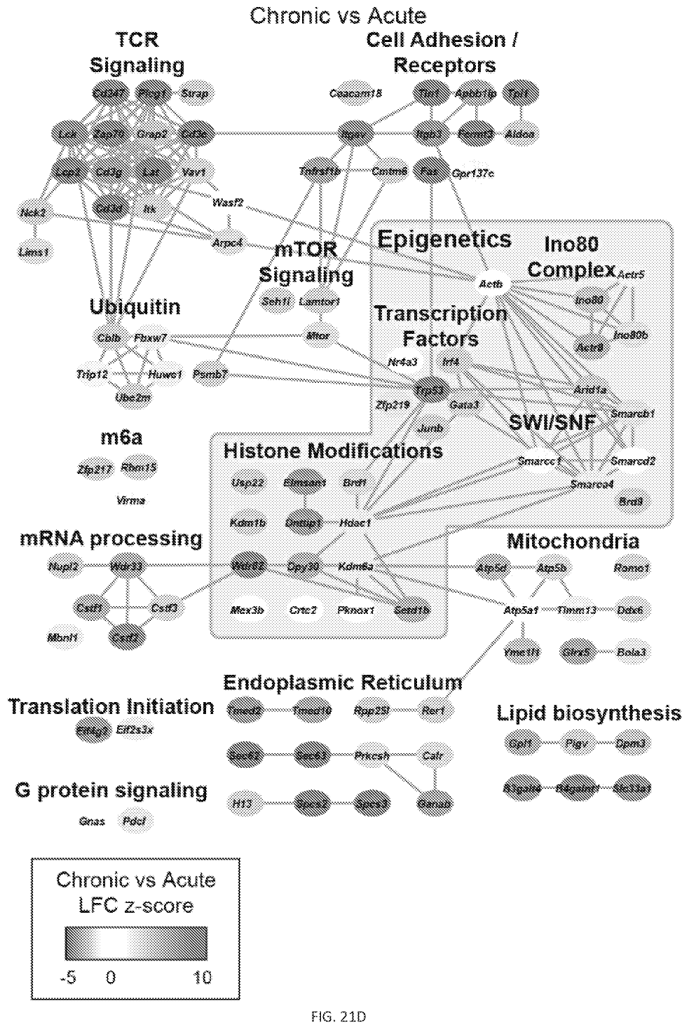

To further validate and characterize the top ranked genome-wide screen factors, a custom mini-pool of 2,000 sgRNAs, which included sgRNAs that targeted 300 top ranked genes (6 sgRNAs per gene), as well as 100 non-targeting and 100 single-targeting controls was created. The in vitro stimulation screen was repeated and acute and chronic samples, as well as input samples were collected on day 4 (FIG. 21A). High concordance between biological replicates was observed and therefore the replicates were merged to perform three comparisons: (1) chronic vs acute, (2) acute vs input, and (3) chronic vs input (FIGS. 21B-21E). The chronic vs acute comparison served as validation of the original genome-wide screen, and of the 88 genes in the pool that were significant positive hits in the genome wide screen, 52 (59.1%) were validated in the mini-pool (FDR<0.05; FIGS. 11B and 21C). Next, the chronic vs acute gene enrichments were compared to acute vs input enrichments, which measured the fitness advantage or disadvantage of each gene knockdown in acutely stimulated proliferating T cells in culture (FIGS. 11C, left and 21E). Two hits. Trp53 and Brd1, were enriched in both comparisons, demonstrating that depletion of these factors imparts an overall proliferation advantage to T cells in both acute and chronic stimulation conditions. In contrast, most genes displayed either similar (233/30; 77.7%) or reduced (64/300; 21.3%) enrichments in acute stimulation compared to input, enabling the identification of sgRNAs that specifically improve T cell persistence in the presence of chronic antigen, rather than T cell proliferation in general, and that maintain proliferative capacity after acute stimulation (Similar: −3.5≤z≤3, reduced: z<−3.5, improved: z>3.5; FIGS. 11C, left and 21E). Finally, chronic vs acute sgRNA enrichments were compared to chronic vs input enrichments to identify sgRNAs that have an overall persistence advantage after chronic antigen stimulation, rather than only a comparative advantage to acute stimulation (FIG. 11C, right). In summary, these mini-pool experiments validated hits from the genome-wide CRISPR screen and identified genes which selectively restrict T cell persistence in the setting of chronic antigen stimulation.

### Example 6

Tuning cBAF Activity can Enhance T Cell Persistence

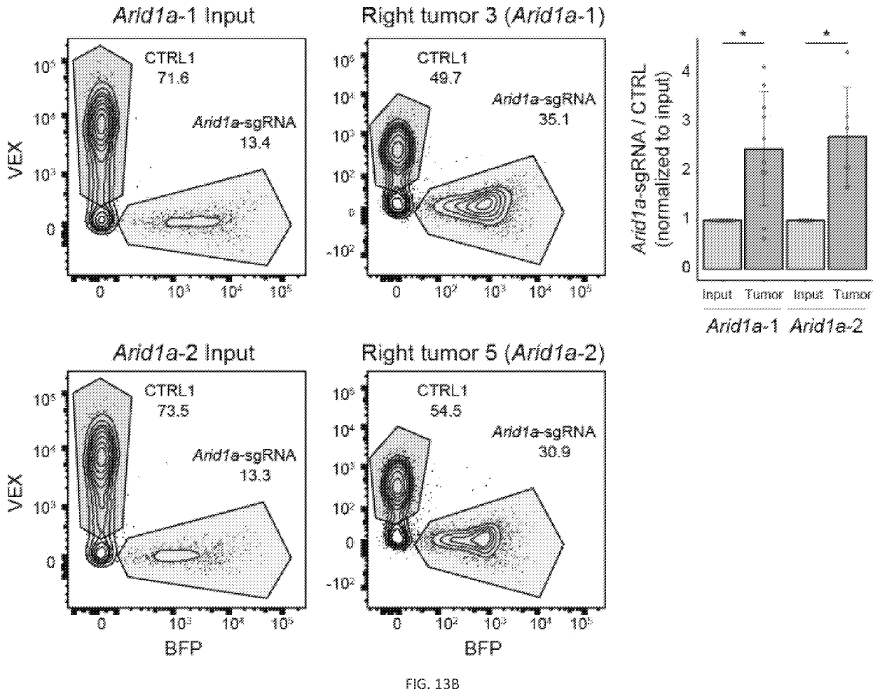

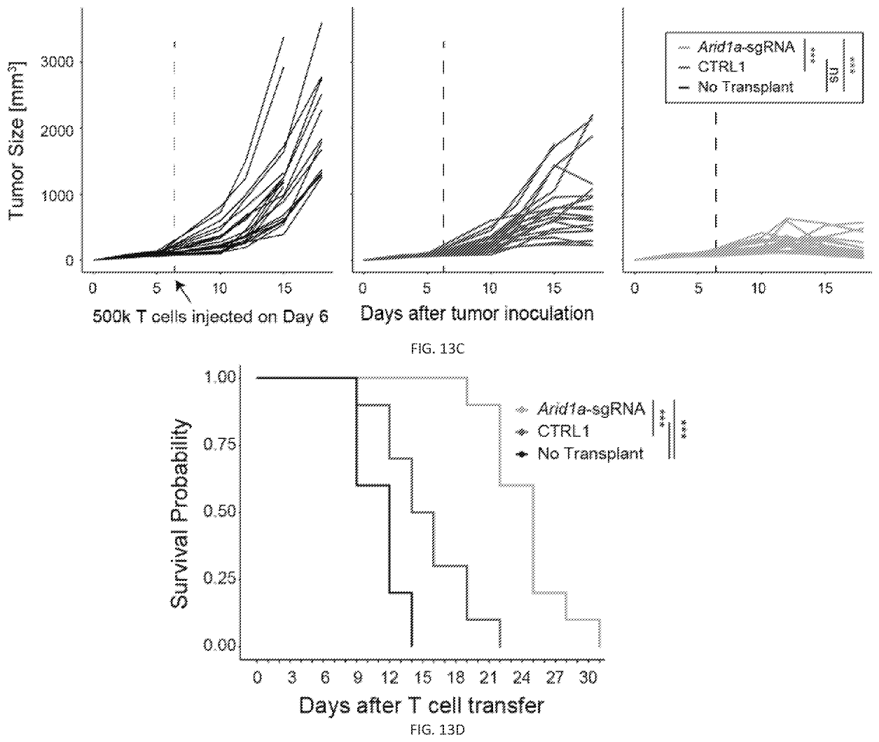

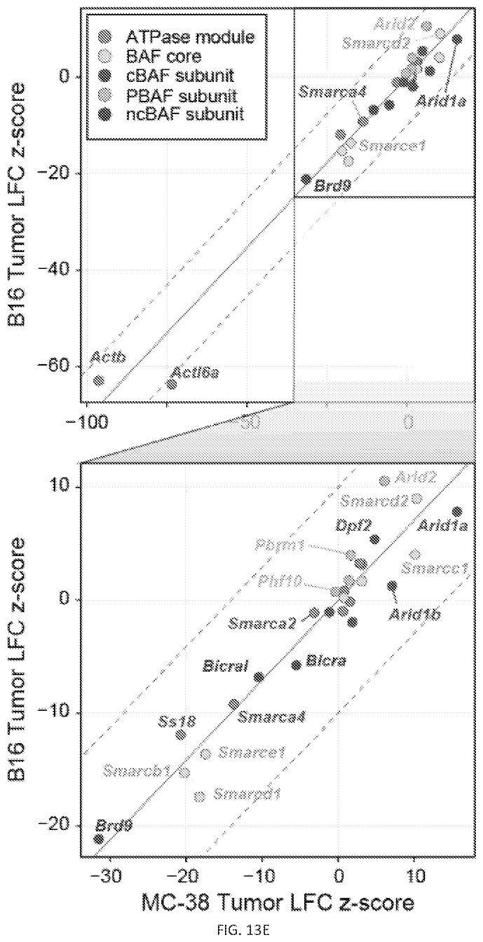

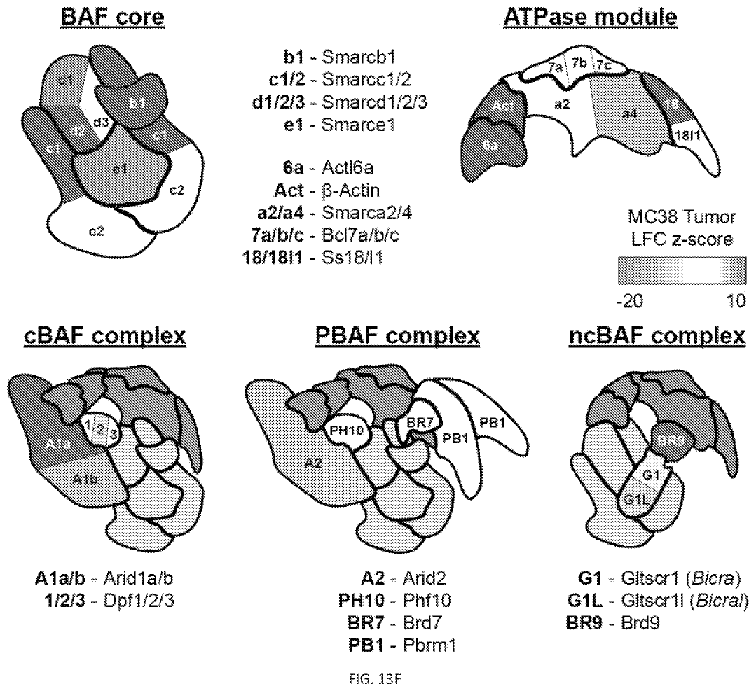

To validate the persistence advantage of Arid1a-sgRNA cells (top hit in the screen) and determine whether these cells retained effector function in vivo, a cell competition assay where a single-targeting control (CTRL1) sgRNA was cloned into a retroviral vector expressing a violet-excited fluorescent protein (VEX), while two Arid1a-sgRNA sgRNAs (Arid1a-1 and Arid1a-2) were cloned into a vector which was identical except for the substitution of a blue fluorescent protein (BFP) was used. The activity of both Arid1a-targeting sgRNAs was confirmed at the DNA and protein level by Sanger sequencing and Western blot (FIGS. 22D-22F). Cells were separately transduced with either vector, selected with puromycin to enrich for transduced cells, and mixed together. The mixed cells were then put into the in vitro chronic stimulation assay (FIG. 13A) or the in vivo MC-38 tumor model (FIG. 13B). In vitro and in vivo, Arid1a-sgRNA cells demonstrated significantly enhanced persistence, compared to control cells, confirming the results of the pooled screens (FIGS. 13A-13B; Arid1a-1 to CTRL1 average normalized ratio: in vitro day 10=4.03, p=0.0059; in vivo day 15=2.46, p=0.033; Arid1a-2 to CTRL1 average normalized ratio: in vitro day 10=3.79, p=0.012; in vivo day 15=2.72, p=0.0088; Welch Two Sample t-test). Moreover, Arid1a-sgRNA cells exhibited lower levels of PD-1 and Tim3 after chronic stimulation in vitro (percentage double positive cells: Arid1a-1 average decrease of 27.7%, p=0.00099; Arid1a-2 average decrease of 10.6%, p=0.038; Welch Two Sample t-test; FIG. 13A). Finally, whether the observed enhanced persistence and altered differentiation trajectory of Arid1a-sgRNA cells resulted in improved anti-tumor responses in vivo was evaluated. Rag1^{-/-} mice were inoculated with MC-38 tumors as previously described, and on day 6, transplanted 5×10⁵ Cas9/OT-1 CD8⁺ T cells transduced with either CTRL1 retrovirus or Arid1a-sgRNA retrovirus and monitored tumor growth (FIG. 13C). By day 15, transfer of Arid1a-sgRNA cells significantly improved tumor clearance, compared to transfer of control cells (Arid1a-sgRNA vs CTRL1 tumor size, Day 15: p=5×10⁻⁸; Welch Two Sample t-test). Importantly, survival of mice receiving Arid1a-sgRNA T cells was significantly extended, compared to mice receiving CTRL1 T cells (median survival=12 days (no transplant), 15 days (CTRL1), 25 days (Arid1a-sgRNA); Arid1a-sgRNA vs CTRL1: p=1.20×10⁻⁸ FIG. 13C).

To provide deeper mechanistic insight into the role of BAF complex factors in T cell exhaustion, an additional CRISPR mini-pool screen targeting each of the 29 SWI/SNF complex subunit genes in the B16 and MC-38 tumor models was designed and these results were interpreted in the structural context of SWI/SNF complex assembly. As observed in the prior in vivo screen, the three most significant hits were in the cBAF complex (Arid1a, Smarcc1, and Smarcd2) and notably were in positions of the complex that can be substituted by paralogs in other forms of the complex (FIGS. 13E-13F). In contrast, perturbation of irreplaceable subunits of the BAF core (e.g., Smarcc1, Smarcb1) or ATPase module components was deleterious and led to depletion of these sgRNAs. Therefore, a model is proposed in which tuning (reducing) the presence of cBAF on chromatin is beneficial for T cell persistence. Prior mechanistic studies demonstrated that ARID1A-deficient tumors exhibit reduced (but not ablated) levels of cBAF complex on chromatin, which resulted in decreased access of key transcription factors (including AP-1 factors). In addition to cBAF, positive enrichments were also observed of sgRNAs targeting the PBAF complex member, Arid2, and strong depletion of sgRNAs targeting the ncBAF complex members, Bicra1, Bicra, and Brd9 (FIGS. 13E-13F). In summary, these results demonstrate that perturbation of cBAF complex subunit genes, including Arid1a, can improve T cell persistence and anti-tumor immunity in vivo.

### Example 7

### Perturbation of ARID1A Improves T Cell Persistence in Primary Human T Cells

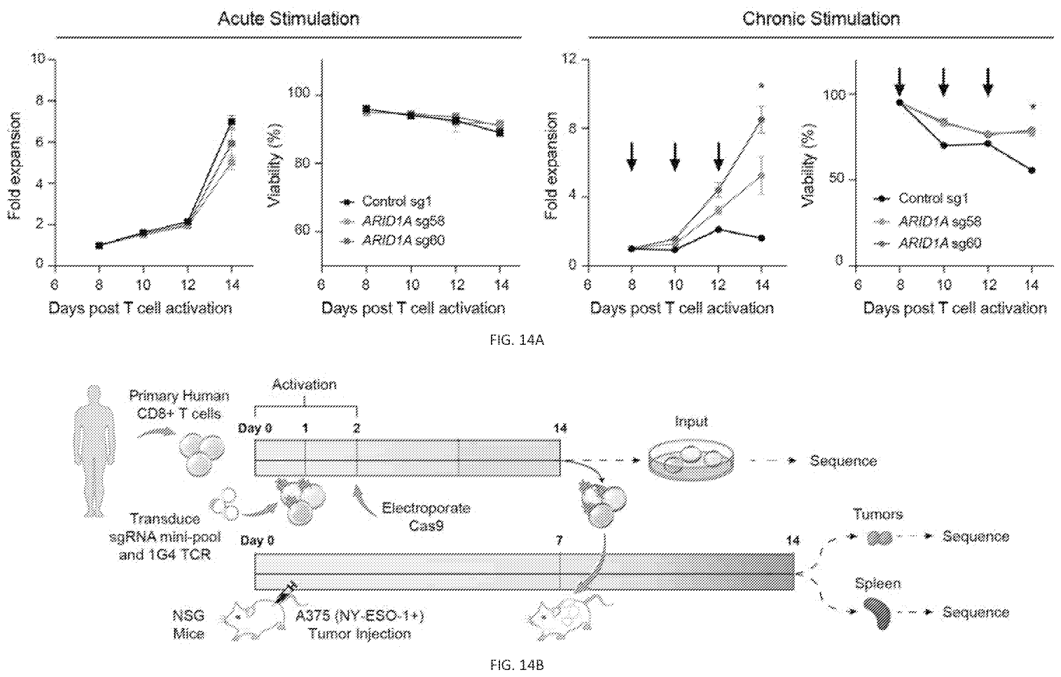

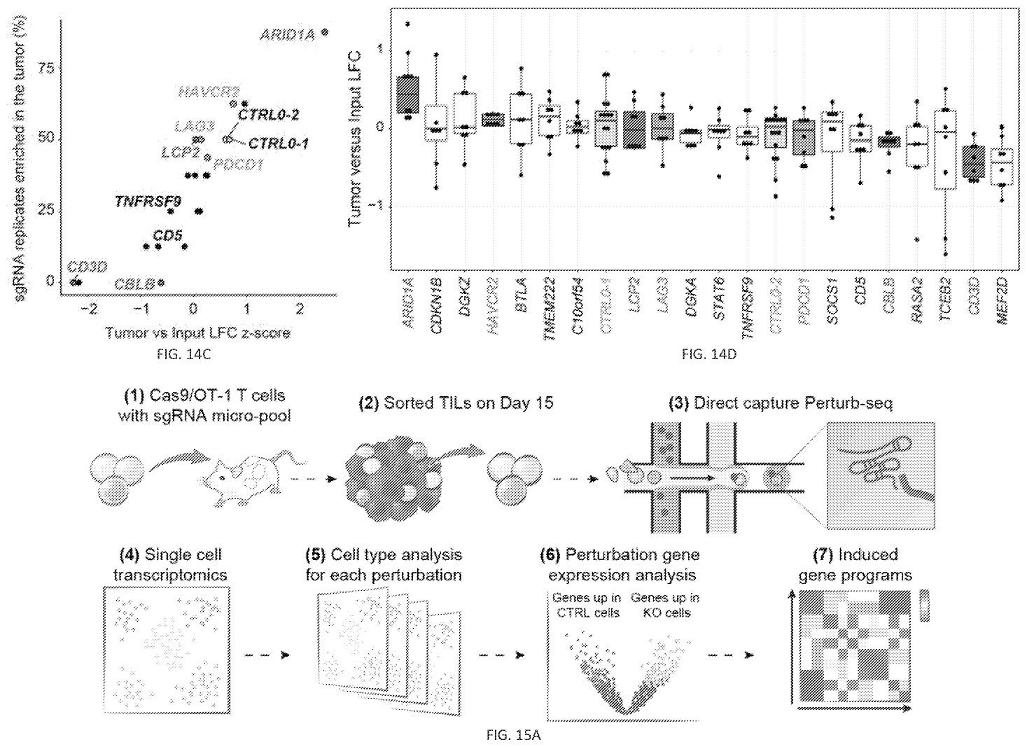

To replicate the in vitro chronic stimulation assay using human T cells (FIG. 14A), CRISPR-Cas9/sgRNA RNPs targeting ARID1A (two independent sgRNAs) or a control RNP were introduced into primary human T cells. The cells were split into acute and chronic cultures, and the chronic condition was stimulated for 6 days with anti-CD3 coated plates (analogous to the mouse assay). In acutely stimulated cultures, no difference between the genotypes for proliferation or viability were observed. However, in chronically stimulated cultures, ARID1A-sgRNA cells proliferated significantly more and maintained higher viability than CTRL T cells (ARID1A-sgRNA vs CTRL1 cells: mean increase of 22.75% viability, p=1.70×10⁻⁵, and mean increase of 5.25-fold expansion, p=0.013; FIG. 14A).

To validate the persistence advantage of ARID1A-sgRNA T cells in vivo and in the context of other genetic factors that have recently emerged from human T cell functional CRISPR screens, a CRISPR mini-pool was designed for in vivo human T cell experiments, which encompassed 48 sgRNAs targeting 20 genes and included 8 negative control guides. sgRNAs targeting ARID1A, as well as the inhibitory receptors, PDCD1, LAG3, and HAVCR2, and other top-ranked genes from prior screens, such as TMEM222, CBLB, TCEB2, and SOCS1 were included. The screen was performed in the A375 human melanoma xenograft model, which expresses the NY-ESO-1 antigen that can be targeted with the 1G4 TCR. The cognate 1G4 TCR was introduced into primary human T cells from two independent donors on day 1 along with the sgRNAs, and on day 14 transplanted T cells into NOD-SCID-IL2Rγ-null (NSG) tumor-bearing mice (FIG. 14B). 7 days later, T cells were sorted from the tumors and spleens, sequenced sgRNAs present in each organ, and compared their abundance to input samples prior to transplant. Enrichments in control sgRNAs or sgRNAs targeting inhibitory receptors were not observed, while depletion of sgRNAs targeting CD3D were observed (FIG. 14C-14D). In contrast, and consistent with results in murine T cells, sgRNAs targeting ARID1A were significantly enriched in tumors compared to input samples in both donors, demonstrating that the function of cBAF in limiting T cell persistence is also conserved in human T cells (7 of 8 ARID1A-sgRNA replicates enriched in tumors versus input, merged across 2 independent sgRNAs; ARID1A-sgRNA versus CTRL LFC p=0.0010 by Wilcoxon test; FIGS. 14C-14D).

### Example 8

### Transcriptional Effects of Chromatin Remodeling Complexes in TILs

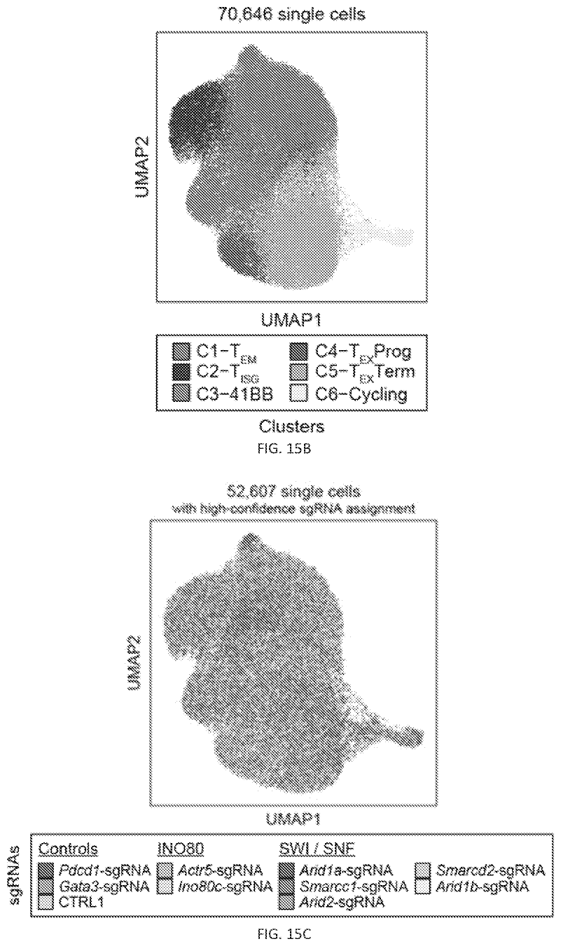

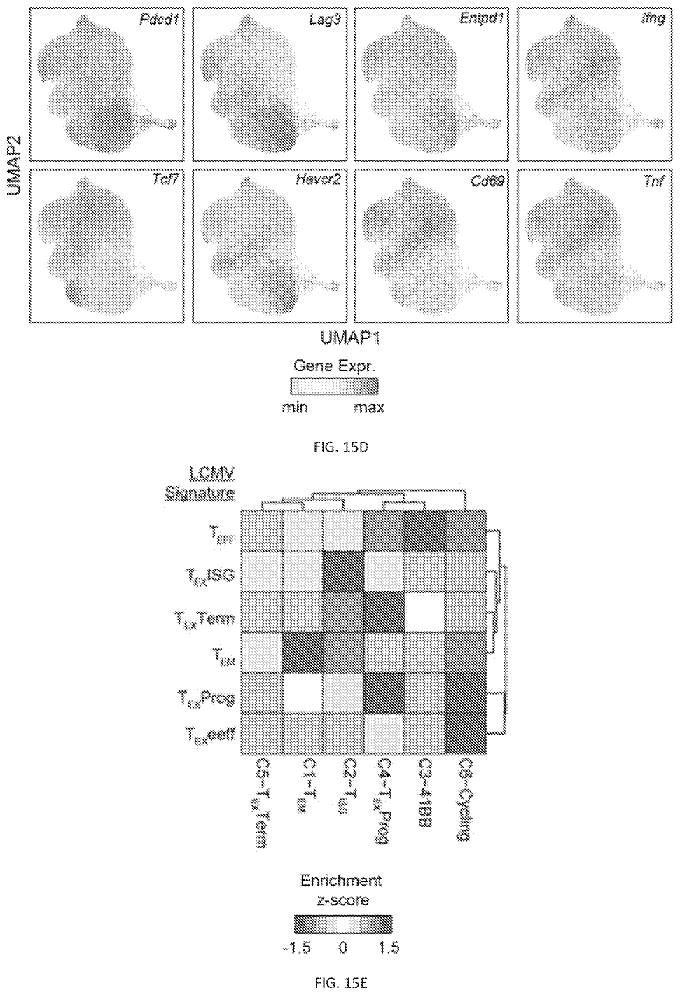

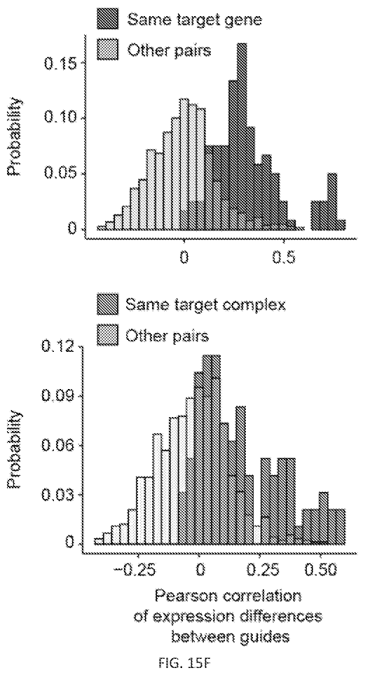

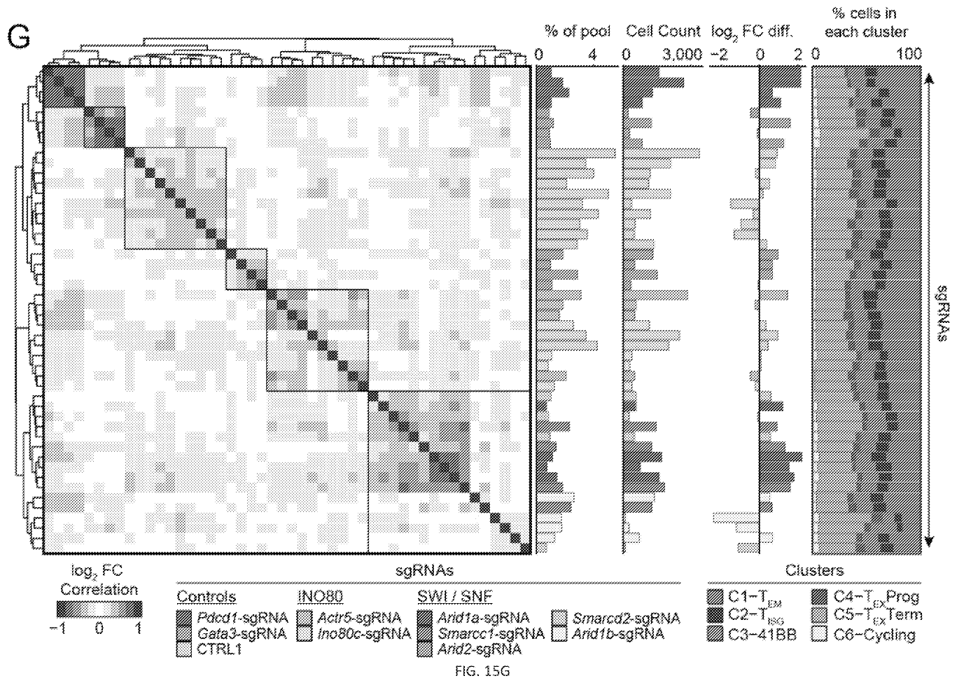

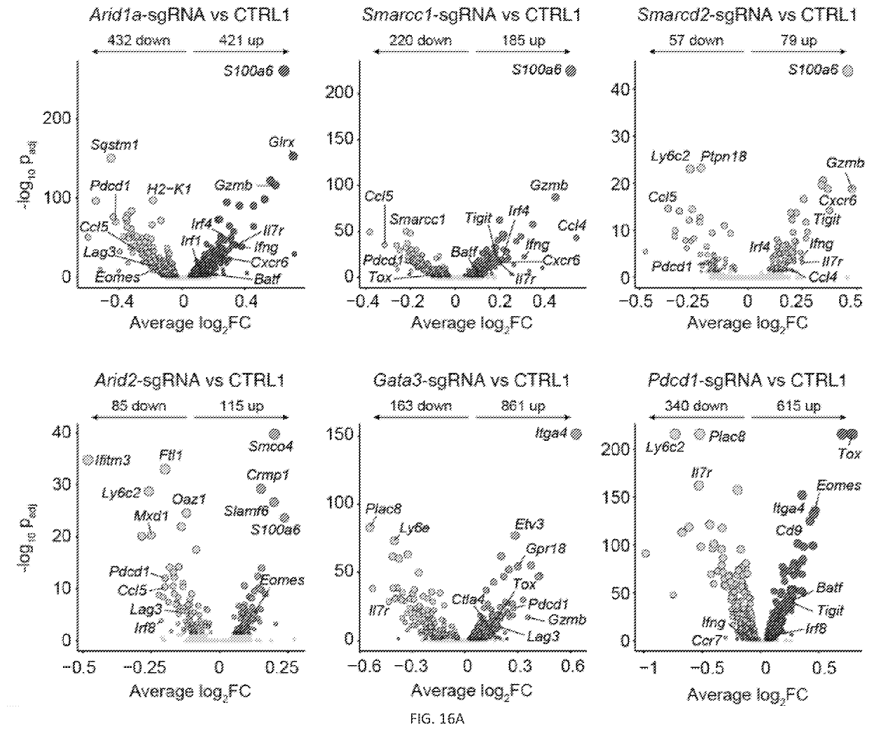

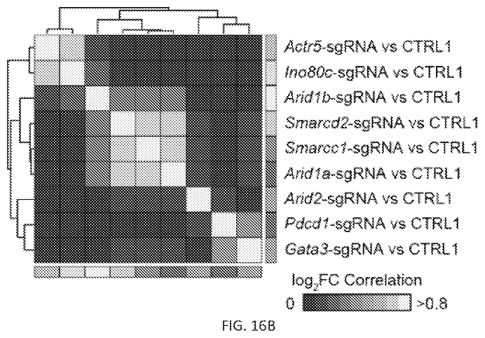

To understand the molecular mechanisms driving improved T cell function in hits identified by the in vitro and in vivo CRISPR screens, Perturb-seq was performed, which simultaneously captures CRISPR sgRNAs and the transcriptome in single cells. A third custom sgRNA pool (micro-pool) was designed targeting the INO80 and BAF complexes. Both complexes are ATP-dependent chromatin remodelers that are essential in many aspects of development. For SWI/SNF genes. Arid1a, Smarcc1, and Smarcd2 (top hits identified in vitro and in vivo), as well as Arid2 and Arid1h, which were enriched in the SWI/SNF-specific mini-pool screen, were targeted. Of these, Smarcc1 and Smarcd2 are in the BAF core, Arid1a and Arid/b are in the cBAF complex, and Arid2 is present only in the PBAF complex. From the INO80 complex, Actr5 and Ino8c, which were enriched in both the in vitro and in vivo screens, were selected. Interestingly, the yeast homologues of Actr5 and Ino80c, Arp5 and Ies6, have been shown to physically associate with each other, forming a subcomplex independent of the rest of the INO80 complex. The subcomplex can modulate the activity of the rest of the INO80 complex; it interacts with chromatin in an INO80-dependent manner and repositions nucleosomes (particularly the +1 nucleosome) to activate gene transcription, especially at metabolism-related genes. Finally, positive controls, Pdcd1 and Gata3, as well as 12 single targeting negative controls were included, for a total of 48 sgRNAs targeting 9 genes. A similar in vivo T cell protocol was performed as described above for the larger CRISPR screen: CD8⁺ T cells were isolated from Cas9/OT-1 mice, transduced with the sgRNA micro-pool, and then transplanted into Rag1^{-/-} mice bearing MC-38 ovalbumin tumors. As in the prior screens, an input sample (collected on the day of transplantation) was also collected to evaluate the persistence phenotype of each sgRNA. Nine days after T cell transplantation, tumors were harvested tumors, TILs were isolated, and direct-capture Perturb-seq was used to read out sgRNA identity and gene expression profiles simultaneously using the 10× Genomics 5′ gene expression platform (FIG. 15A). Cells from seven biological replicate Perturb-seq samples across two independent experiments were sequenced (FIGS. 23-23B).

After quality control filtering, high-quality scRNA-seq profiles were obtained from 70,646 cells, and scRNA-seq clustering and dimensionality reduction identified 6 clusters (FIG. 15B). A high-confidence sgRNA identity was identified for each cell by using z-scores to quantify the enrichment of each sgRNA, relative to other sgRNAs detected in the same cell. Cells were assigned to a particular sgRNA if that sgRNA had a z-score of at least 5, and a z-score at least 2 units higher than the next-most prevalent sgRNA. With this strategy, cells with multiple enriched sgRNAs due to retroviral infection doublets, single-cell capture doublets, and/or background reads were removed from further analysis, and 52,607 cells were confidently assigned to a single sgRNA (74.4%; FIG. 15C). Cell type clusters expressed varied levels of inhibitory receptors, effector cytokines, and key transcription factors, indicating that they represented a mix of exhausted and effector T cells in the TME (FIGS. 15D and 23C-23D). Cluster 1 cells expressed high levels of Klf2 and Slpr1 (T effector memory; T_{EM}), Cluster 2 expressed high levels of interferon stimulated genes (ISGs) including Mx1 (T_{ISG}), Cluster 3 expressed high levels of Tnfrsf9 (encoding 41BB) and Cd160 (T-41BB), Cluster 4 expressed high levels of progenitor exhaustion genes including Pdcd1, Tcf7 and Slamf6 (T_{EX}Prog), Cluster 5 expressed the highest levels of inhibitory receptors Pdcd1, Lag3, and Havcr2 (T_{EX}Term), and Cluster 6 consisted primary of cycling cells, marked by Mki67 and confirmed by cell cycle analysis (T-Cycling; FIGS. 23C-23D). To further refine the cluster identities, gene signatures were generated from CD8⁺ T cell types present in acute or chronic LCMV infection in vivo (FIGS. 23E-23F). The top 100 marker genes were used for each LCMV T cell cluster to score each single cell in our Perturb-seq dataset according to the average expression of these signature gene sets. Visualizing the enrichment of these LCMV signatures in each cluster demonstrated transcriptional similarity of several clusters to cell types in the reference dataset (FIG. 15E). For example. Cluster 1 was enriched for the effector memory-related genes (T_{EM} signature), Cluster 2 was similar to the T_{EX}ISG signature, and the progenitor and terminally exhausted clusters (Clusters 4 and 5) enriched the corresponding LCMV signatures (FIG. 15E).

Several sgRNA-level quality controls were performed to assess the reproducibility of effects of independent sgRNAs (FIG. 15F-15G). Gene expression differences were computed between each sgRNA and all other cells in the dataset and confirmed that independent sgRNAs targeting the same gene had highly correlated gene expression changes relative to pairs of sgRNAs targeting different genes, which, as expected, centered around zero (FIGS. 15F, top). The correlation of pairs of sgRNAs targeting the same complex was evaluated, grouping together guides targeting cBAF genes (Arid1a, Arid1b, Smarcc1, and Smarcd2) and guides targeting INO80 genes (Ino80c or Actr5). Strikingly, these sgRNA pairs were also significantly more correlated than all pairs of guides, indicating common transcriptional effects of targeting distinct subunits within the same complex (FIG. 15F, bottom). Gene expression correlations of all pairs of sgRNAs were visualized together (FIG. 15G). Unbiased clustering organized sgRNAs into correlated groups, primarily driven by target gene and target complex identity. Interestingly, Arid2 clustered separately from the rest of the BAF targeted sgRNAs, suggesting distinct roles for the cBAF and PBAF complexes (FIG. 15G). The input representation of each sgRNA along with the number of cells detected with each sgRNA was used to estimate the T cell accumulation advantage between each sgRNA and the set of single targeting negative controls (FIG. 15G). This analysis demonstrated that the majority of sgRNAs enhanced T cell accumulation in the tumor, relative to control sgRNAs, in line with the in vivo screen results. In particular, Arid1a-sgRNA cells were enriched 2.74-fold on average relative to controls, while Pdcd1-sgRNA cells were enriched 2.67-fold on average relative to controls (FIG. 15G). Finally, the cell type cluster composition of cells containing each sgRNA was examined (FIG. 15G, far right). Notably, all perturbations contained cells from each cluster with similar proportions, suggesting that depletion of each target gene may not impact wholescale changes in cell type compositions or trajectories, but rather modulate gene expression in one or more clusters.

To further investigate this possibility, cells that contained sgRNAs targeting the same gene were aggregated and differential gene expression was computed for each perturbation, compared to CTRL1 cells (FIG. 16A). Targeting cBAF subunits Arid1a, Smarcd2, or Smarcc1 induced shared global changes in the transcriptional program of T cells, including the upregulation of effector molecules, Gzmb and Ifng, cell surface receptors, Cxcr6 and Il7r, and transcription factors, Irf4 and Batf. Meanwhile, Pdcd1, Lag3, and Ccl5 were consistently downregulated by cBAF perturbation (FIG. 16A). In contrast, Arid2 perturbation induced a distinct gene expression program, albeit with some similarities, including the downregulation of Pdcd1 and Lag3. Perturbation of Gata3 and Pdcd1 induced distinct gene expression changes from either cBAF or Arid2 perturbation; for example, the most upregulated gene after Pdcd1 depletion was Tox, perhaps consistent with the proposed impact of PD-1 deletion on accelerating differentiation to terminal exhaustion (FIG. 16A). To quantify the aggregate similarity of gene expression changes induced by each perturbation, all pairs of perturbations were correlated and clustering was performed to group perturbations that were similar according to this metric (FIG. 16B). This analysis quantitatively confirmed the observation that cBAF perturbations, Arid1a, Smarcc1, and Smarcd2, induced similar programs, while INO80 perturbations, Ino80c and Actr5, also exhibited highly correlated changes (that are distinct from that induced by cBAF perturbation). In contrast, Pdcd1 and Gata3 perturbations clustered separately, although with moderate correlation to each other. Finally, when gene expression changes were considered for perturbed vs CTRL1 cells within each cluster, each perturbation induced highly concordant changes in gene expression regardless of the T cell subtype (FIG. 23G).

All genes significantly differential between perturbed cells and CTRL1 cells were aggregated and core gene programs perturbed by depletion of the cBAF and INO80 complexes were defined (FIGS. 16C-16E). The upregulated and downregulated gene sets were highly conserved within each complex (FIGS. 16D-16F and 24A), with cBAF perturbation inducing genes such as Batf, Irf4, 117r, and Ccr2, while repressing genes such as Stat3, Nfkb1, Nr4a3, and Eomes. In contrast, INO80 perturbation substantially modulated metabolism related genes (FIG. 16E). Projection of genes upregulated by cBAF depletion onto canonical T cell states identified in chronic LCMV infection showed an enrichment in effector T cell genes, while projection of downregulated genes showed an enrichment in terminal exhaustion-related genes (FIGS. 16G and 24B). GO Term analysis of upregulated gene sets was performed. Genes upregulated in cBAF-deficient T cells enriched effector terms, including T cell activation, cell adhesion, cytokine production, and T cell proliferation, while genes upregulated in INO80-deficient T cells enriched metabolic terms, including oxidative phosphorylation and aerobic respiration (FIG. 16H). In contrast, perturbation of Pdcd1 induced cell signaling related terms (FIG. 16H). These data demonstrated that subunits of the cBAF and INO80 chromatin remodeling complexes have distinct roles in T cell exhaustion that are largely conserved within the same complex, with cBAF primarily regulating effector- and exhaustion-related genes and INO80 regulating metabolism. Furthermore, the transcriptional impact of targeting chromatin remodeling factors minimally overlaps with the impact of previously known targets, Pdcd1 and Gata3, suggesting the potential to synergistically target multiple pathways to improve T cell function (FIGS. 16F and 24A).

### Example 9

Terminal Exhaustion-Associated Chromatin Accessibility with Arid1a Perturbation

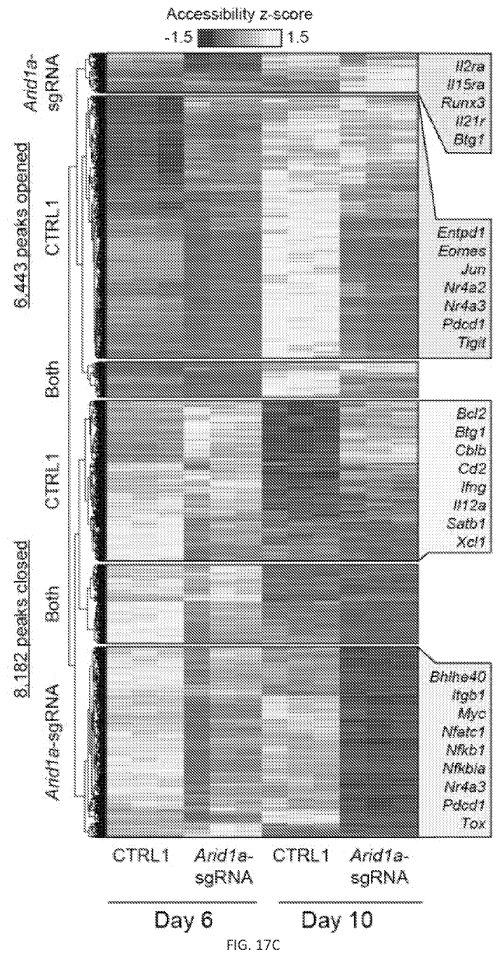

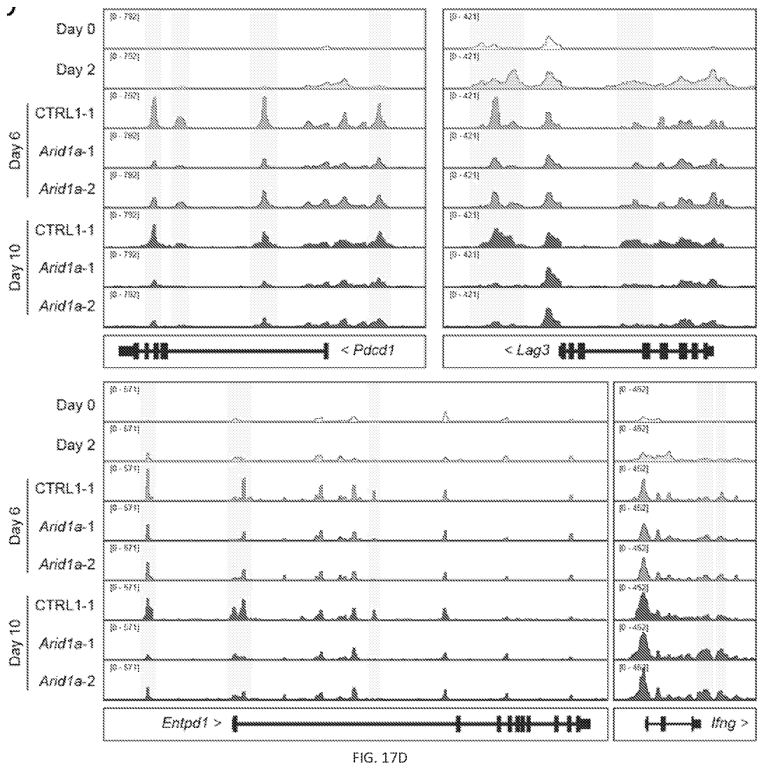

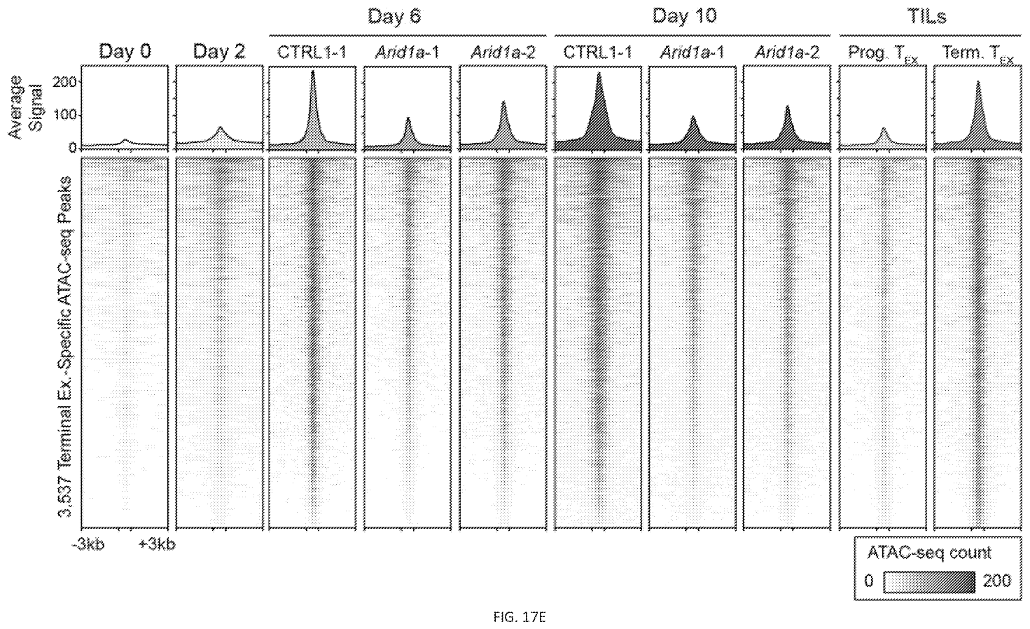

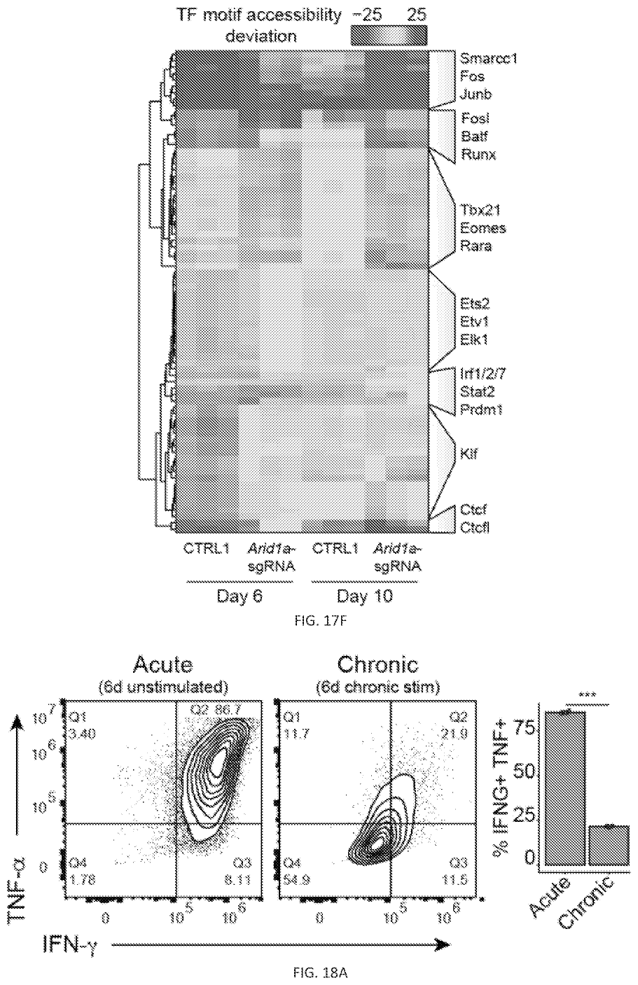

A competition assay was performed as described above, wherein CTRL1 and Arid1a-sgRNA cells were mixed at a defined ratio and subjected to in vitro exhaustion. Two independent sgRNAs targeting Arid1a were used in duplicates for a total of four replicate samples. At Day 6 and Day 10, CTRL1 and Arid1a-sgRNA cells were isolated from the same culture and ATAC-seq was performed on each population. To analyze these results in the context of the initial assay characterization (FIG. 1), the profiles of naïve (Day 0) and activated (Day 2) WT T cells were included (FIG. 17A). The chromatin state progression in CTRL1 cells proceeded similarly to that observed previously in unperturbed cells; however, Arid1a-sgRNA cells proceeded down a distinct chromatin state trajectory, remaining closer to naïve and activated samples than the CTRL1 cells at both time points (FIG. 17A).

Regulatory elements were defined as ‘opened’ peaks if increased accessibility at Day 10, compared to Day 6, was observed, and as ‘closed’ peaks if decreased accessibility at Day 10, compared to Day 6, was observed (p_{adj}<0.05, Log₂ FC>1). Analysis of these peak sets demonstrated substantially different chromatin remodeling changes in Arid1a-sgRNA T cells, compared to CTRL1 T cells (FIGS. 17B-17C). First, Arid1a-sgRNA cells exhibited a marked global decrease in the number of opened peaks, likely representing a relative inability of cBAF-depleted cells to establish accessible chromatin (Arid1a-sgRNA:1.419 peaks. CTRL 1: 5,692 peaks; FIG. 17B). Second, while Arid1a-sgRNA cells and CTRL1 cells closed chromatin to a similar extent, the majority of these regions were non-overlapping (Arid1a-sgRNA: 5,126 peaks, CTRL 1: 4,558 peaks; FIG. 17B). Inspection of individual exhaustion-associated regulatory elements, including those surrounding Pdcd1, Lag3, Entpd1, and Ifng gene loci, revealed a substantial loss of accessibility in Arid1a-sgRNA cells, compared to CTRL1 cells (FIG. 17D). Analysis of the terminal T_{EX}-specific peak set (as defined in FIG. 1) showed that these sites were significantly less accessible in Arid1a-sgRNA cells than in CTRL1 cells at both time points (mean decrease in accessibility of Terminal T_{EX} peaks in Arid1a-sgRNA relative to CTRL1 cells: 41.7% on Day 6 (p<2.2×10⁻⁶, Wilcoxon Test) and 40.8% on Day 10 (p<2.2×10¹⁶, Wilcoxon Test); FIGS. 17E and 24C-24D). Chromatin accessibility at TF binding sites was analyzed using chromVAR, which showed that terminal exhaustion-associated TF motifs, including Fos, Jun, and AP-1 motifs were significantly less accessible in Arid1a-sgRNA cells, compared to CTRL1 cells (FIG. 17F). Conversely, several TF motifs associated with effector T cell function, including Ets, Klf, and Irf motifs, showed increased accessibility in Arid1a-sgRNA cells. Finally, ATAC-seq analysis of chronically stimulated ARID1A-sgRNA human T cells demonstrated a similar loss of global chromatin accessibility at AP-1 motifs, compared to control T cells, supporting the conserved epigenetic function of ARID1A in human T cells (FIGS. 24E-24G). These results suggested that depletion of cBAF subunits, including Arid1a, may improve T cell function by restricting the access of AP-1 TFs to chromatin and thereby preventing the acquisition of the terminal exhaustion-associated chromatin state.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An engineered T cell lacking at least one gene selected from the group consisting of: INO80C, GATA3, ARID1A, WDR82, TRP53, GPR137C, ZFP219, HDAC1, ELMSAN1, and ACTR8.

2. The engineered T cell of claim 1, wherein the engineered T cell lacks two or more genes selected from the group consisting of: INO80C, GATA3, ARID1A, WDR82, TRP53, GPR137C, ZFP219, HDAC1, ELMSAN1, and ACTR8.

3. An engineered T cell lacking at least one chromatin remodeling protein or a gene encoding thereof.

4. The engineered T cell of claim 3, wherein the engineered T cell lacks two or more chromatin remodeling proteins or genes encoding thereof

5. The engineered T cell of claim 3 or 4, wherein the at least one chromatin remodeling protein is a INO80 nucleosome positioning complex protein or SWI/SNF family member, or a combination thereof.

6. The engineered T cell of claim 5, wherein the INO80 nucleosome positioning complex protein is Actr5, Ino80, Ino80c, Ino80b, Actr8, or a combination thereof.

7. The engineered T cell of claim 5 or 6, wherein the SWI/SNF family member is a member of cBAF complex

8. The engineered T cell of any of claims 5-7, wherein the SWI/SNF family member is Arid1a, Arid2, Arid1b, Smarcb1, Smarcd2, Smarca4, Smarcc1, or a combination thereof.

9. The engineered T cell of any of claims 3-8, wherein the engineered T cell further lacks at least one gene selected from the group consisting of: GATA3, WDR82, TRP53, GPR137C, ZFP219, HDAC1, and ELMSAN1.

10. The engineered T cell of any of claims 1-9, wherein the engineered T cell maintains functionality under conditions in which non-engineered T cells display exhaustion.

11. The engineered T cell of any of claims 1-10, further comprising an exogenous receptor or a nucleic acid encoding thereof.

12. The engineered T cell of claim 11, wherein the exogenous receptor is a T cell receptor (TCR) or chimeric antigen receptor (CAR).

13. The engineered T cell of claim 11 or 12, wherein the exogenous receptor is specific for a tumor antigen.

14. The engineered T cell of any of claims 1-13, wherein the T cell is derived from a biological sample from a subject.

15. The engineered T cell of claim 14, wherein the T cell is isolated from a tumor sample.

16. The engineered T cell of any of claims 1-15, wherein the T cell is expanded ex vivo.

17. A composition comprising a population of engineered T cells of any of claims 1-16.

18. The composition of claim 17, further comprising at least one therapeutic agent.

19. The composition of claim 18, wherein the at least one therapeutic agent is selected from the group consisting of: an agent for treating T cell exhaustion; an antiviral agent; an antibiotic agent; an antimicrobial agent; a chemotherapeutic agent; or a combination thereof.

20. A method of making a therapeutic T cell, comprising the steps of:
obtaining a sample comprising a T cell;
altering the DNA of the T cell to knockout or disrupt at least one gene selected from the group consisting of: INO80C, GATA3, ARID1A, WDR82, TRP53, GPR137C, ZFP219, HDAC1, ELMSAN1, and ACTR8; and
engineering the T cell to express an exogenous receptor.

21. A method of making a therapeutic T cell, comprising the steps of:
obtaining a sample comprising a T cell;
altering the DNA of the T cell to knockout or disrupt at least one gene encoding a chromatin remodeling protein; and
engineering the T cell to express an exogenous receptor.

22. The method of claim 21, wherein the at least one chromatin remodeling protein is a INO80 nucleosome positioning complex protein or SWI/SNF family member, or a combination thereof.

23. The method of claim 22, wherein the INO80 nucleosome positioning complex protein is Actr5, Ino80, Ino80c, Ino80b, Actr8, or a combination thereof.

24. The method of claim 22 or 23, wherein the SWI/SNF family member is a member of cBAF complex

25. The method of any of claims 22-24, wherein the SWI/SNF family member is Arid1a, Arid2, Arid1b, Smarcb1, Smarcd2, Smarca4, Smarcc1, or a combination thereof.

26. The method of any of claims 21-25, wherein the method further comprising altering the DNA of the T cell to knockout or disrupt at least one gene selected from the group consisting of: GATA3, WDR82, TRP53, GPR137C, ZFP219, HDAC1, and ELMSAN1.

27. The method of any of claims 20-26, wherein the T cell is from a subject.

28. The method of any of claims 20-27, wherein the T cell is isolated from a tumor sample.

29. The method of claim 27 or 28, further comprising expanding the T cell ex vivo.

30. The method of any of claims 20-29, wherein the exogenous receptor is a T cell receptor (TCR) or chimeric antigen receptor (CAR).

31. The method of claim 30, wherein the exogenous receptor is specific for a tumor antigen.

32. The method of any of claims 20-31, wherein altering the DNA prevents or reduces exhaustion of the T cell.

33. A method for treating a disease or disorder in a subject comprising administering to the subject an effective amount of the engineered T cells of any of claims 1-16 or a composition of any of claims 17-19.

34. The method of claim 33, wherein the disease or disorder comprises an infection or cancer.

35. The method of claim 34, wherein the cancer comprises a tumor.

36. The method of any of claims 33-35, wherein the administering reduces the number of cancerous cells in the subject.

37. The method of any of claims 33-36, wherein the administering reduces and/or eliminates the tumor burden in the subject.

38. The method of any of claims 33-37, wherein the administering shows enhanced cancer treatment compared to administration of unmodified T cells.

39. The method of any of claims 33-38, further comprising administering at least one additional therapeutic agent.

40. The method of claim 39, wherein the at least one therapeutic agent is selected from the group consisting of: an agent for treating T cell exhaustion; an antiviral agent; an antibiotic agent; an antimicrobial agent; a chemotherapeutic agent; or a combination thereof.

41. The method of any of claims 33-40, wherein the engineered T cells are autologous to the subject.

42. The method of any of claims 33-41, wherein the engineered T cells maintain functionality under conditions in which non-engineered T cells display exhaustion.

43. The method of any of claims 33-42, wherein the engineered T cells have improved persistence and function compared to non-modified T cells.

44. A method of preventing T cell exhaustion comprising genetically modifying the T cell to lack at least one gene selected from the group consisting of: INO80C, GATA3, ARID1A, WDR82, TRP53, GPR137C, ZFP219, HDAC1, ELMSAN1, and ACTR8.

45. A method of preventing T cell exhaustion comprising genetically modifying the T cell to lack at least one chromatin remodeling protein or a gene encoding thereof.

46. The method of claim 45, wherein the at least one chromatin remodeling protein is a INO80 nucleosome positioning complex protein or SWI/SNF family member, or a combination thereof.

47. The method of claim 46, wherein the INO80 nucleosome positioning complex protein is Actr5, Ino80, Ino80c, Ino80b, Actr8, or a combination thereof.

48. The method of claim 46 or 47, wherein the SWI/SNF family member is a member of cBAF complex.

49. The method of any of claims 46-48, wherein the SWI/SNF family member is Arid1a, Arid2, Arid1b, Smarcb1, Smarcd2, Smarca4, Smarcc1, or a combination thereof.

50. The method of any of claims 46-49, wherein the method further comprises altering the DNA of the T cell to knockout or disrupt s at least one gene selected from the group consisting of: GATA3, WDR82, TRP53, GPR137C, ZFP219, HDAC1, and ELMSAN1.

51. The method of any of claims 44-50, wherein the T cell further comprises an exogenous receptor or a nucleic acid encoding thereof.

52. The method of claim 51, wherein the exogenous receptor is a T cell receptor (TCR) or chimeric antigen receptor (CAR).

53. The method of claim 51 or 52, wherein the exogenous receptor is specific for a tumor antigen.

54. The method of any of claims 44-53, wherein the T cells have increased survival in the presence of a chronic antigen.

55. The method of any of claims 44-54, further comprising administering the T cells to a subject in need thereof.

56. The method of claim 55, wherein the subject has cancer or an infectious disease.

57. A method for screening for genes which facilitate T cell exhaustion comprising:
culturing T cells under conditions of chronic or acute stimulation for at least six days, wherein each of T cells comprises at least one gene knockout or knockdown;
isolating T cells not showing an exhausted T cell surface phenotype; and
identifying the at least one gene knockout or knockdown.

58. The method of claim 57, wherein the T cells are a T cell library, wherein the T cell library comprises at least one T cell with a knockout or knockdown for each gene in the genome of the T cell.

59. The method of claim 57 or 58, wherein the T cells are generated using a CRISPR-Cas system wherein each cell comprises at least one guide RNA directed to a gene of interest.

60. The method of any of claims 57-59, wherein the T cells are CD8+ T cells.

61. The method of any of claims 57-60, wherein the T cells are isolated from a subject.

62. The method of any of claims 57-61, wherein conditions of chronic stimulation comprise culturing the T cells using anti-CD3 coated plates.

63. The method of claim 62, wherein the conditions of chronic stimulation further comprise culturing the T cells with IL-2.

64. The method of any of claims 57-63, wherein conditions of acute stimulation comprise culturing the T cells with IL-2.

65. The method of any of claims 57-64, wherein the culturing lasts 6-10 days.

66. The method of any of claims 57-65, wherein the exhausted T cell surface phenotype comprises increased concentrations of TIM-3 and PD-1.

67. Use of the engineered T cells of any of claims 1-16 or a composition of any of claims 17-19 for treating a disease or disorder in a subject.

68. The use of claim 67, wherein the disease or disorder comprises an infection or cancer.

69. The use of claim 68, wherein the cancer comprises a tumor.

70. The use of any of claims 67-69, wherein the number of cancerous cells in the subject is reduced.

71. The use of any of claims 67-70, wherein the tumor burden in the subject is reduced and/or eliminated.

72. The use of any of claims 67-71, wherein the use shows enhanced cancer treatment compared to use of unmodified T cells.

73. The use of any of claims 67-72, further comprising at least one additional therapeutic agent.

74. The use of claim 73, wherein the at least one therapeutic agent is selected from the group consisting of: an agent for treating T cell exhaustion; an antiviral agent; an antibiotic agent; an antimicrobial agent; a chemotherapeutic agent; or a combination thereof.

75. The use of any of claims 67-74, wherein the engineered T cells are autologous to the subject.

76. The use of any of claims 67-75, wherein the engineered T cells maintain functionality under conditions in which non-engineered T cells display exhaustion.

77. The use of any of claims 67-76, wherein the engineered T cells have improved persistence and function compared to non-modified T cells.

78. A system or kit comprising the engineered T cells of any of claims 1-16 or a system for genetic engineering T cells.

79. The system or kit from claim 78, wherein the system for genetic engineering T cells comprises a clustered interspersed short palindromic repeat (CRISPR)/CRISPR-associated protein (Cas) system.

80. The system or kit of claim 78 or 79, wherein the system for genetic engineering T cells comprises Cas9, or a nucleic acid encoding Cas9, and a gRNA directed to at least one gene which facilitates T cell exhaustion, or a nucleic acid encoding the gRNA.

81. The system or kit of claim 80, wherein the at least one gene is selected from the group consisting of: INO80C, GATA3, ARID1A, WDR82, TRP53, GPR137C, ZFP219, HDAC1, ELMSAN1, and ACTR8.

82. The system or kit of claim 80, wherein the at least one gene encodes a chromatin remodeling protein.

83. The system or kit of claim 82, wherein the at least one chromatin remodeling protein is a INO80 nucleosome positioning complex protein or SWI/SNF family member, or a combination thereof.

84. The system or kit of claim 83, wherein the INO80 nucleosome positioning complex protein is Actr5, Ino80, Ino80c, Ino80b, Actr8, or a combination thereof.

85. The system or kit of claim 83 or 84, wherein the SWI/SNF family member is a member of cBAF complex

86. The system or kit of any of claims 83-85, wherein the SWI/SNF family member is Arid1a, Arid2, Arid1b, Smarcb1, Smarcd2, Smarca4, Smarcc1, or a combination thereof.

87. The system or kit of any of claims 78-86, further comprising an exogenous receptor or a nucleic acid encoding thereof.

88. The system or kit of any of claims 78-87, further comprising at least one additional therapeutic agent.

89. The system or kit of claim 88, wherein the at least one therapeutic agent is selected from the group consisting of: an agent for treating T cell exhaustion; an antiviral agent; an antibiotic agent; an antimicrobial agent; a chemotherapeutic agent; or a combination thereof.


SEQUENCE LISTING (7 sequences)

SEQ ID NO: 1 - protein, 8 aa, synthetic construct
SIINFEKL

SEQ ID NO: 2 - DNA, 20 bp, synthetic construct
  source 1..20: mol_type = other DNA
gcagctgcgaagatatcggg

SEQ ID NO: 3 - DNA, 20 bp, synthetic construct
  source 1..20: mol_type = other DNA
cagcagaactcgcacgacca

SEQ ID NO: 4 - DNA, 20 bp, synthetic construct
  source 1..20: mol_type = other DNA
cttactcgacgaatgagccc

SEQ ID NO: 5 - RNA, 20 nt, synthetic construct
  source 1..20: mol_type = other RNA
ggggccactagggacaggat

SEQ ID NO: 6 - RNA, 20 nt, synthetic construct
  source 1..20: mol_type = other RNA
cctgttgaccatacccgctg

SEQ ID NO: 7 - RNA, 20 nt, synthetic construct
  source 1..20: mol_type = other RNA
tgtggctgctgctgatacga